(19) United States
(12) Patent Application Publication
(10) Pub. No.: US 2010/0291537 A1
(43) Pub. Date: Nov. 18, 2010

(54) METHODS AND COMPOSITIONS RELATED TO PHAGE-NANOPARTICLE ASSEMBLIES

(76) Inventors: Glauco Souza, Houston, TX (US); Wadih Arap, Houston, TX (US); Renata Pasqualini, Houston, TX (US); J. Houston Miller, Barnesville, MD (US)

Correspondence Address: FULBRIGHT & JAWORSKI L.L.P., 600 CONGRESS AVE., SUITE 2400, AUSTIN, TX, 78701, US

(21) Appl. No.: 11/719,508

(22) PCT Filed: Nov. 16, 2005
(86) PCT No.: PCT/US05/41700

§ 371 Date: Jan. 26, 2010

Related U.S. Application Data
(60) Provisional application No. 60/628,472, filed on Nov. 16, 2004.

(51) Int. Cl. C12Q 1/70; C12M 1/34

(52) U.S. Cl. 435/5; 435/288.3

(57) ABSTRACT

Embodiments of the invention include additional compositions and related methods and devices for the use of phage-nanoparticle assemblies. Embodiments of the invention include compositions, methods and devices related to phage-nanoparticle assemblies and their use in a variety of methods including detection methods, in vitro and in vivo diagnostic methods, direct and/or indirect therapeutic methods, or combinations thereof. Phage-nanoparticle assemblies of the invention comprise a plurality of nanoparticles complexed with one or more phage particles to form a phage-nanoparticle assembly. In certain aspects, the phage-nanoparticle assembly may also include other agents, including but not limited to organizing agents and/or therapeutic agents.

This application claims priority to U.S. Provisional Patent application Ser. No. 60/628,472, filed Nov. 16, 2004, which is incorporated herein by reference in its entirety.

Support for this application was provided by an award from Gillson-Longenbaugh Foundation.

### FIELD OF THE INVENTION

The present compositions, methods, and devices relate to the fields of medicine, cellular biology, and nanotechnology. More particularly, the compositions, methods, and devices of the invention related to making and using phage-nanoparticle assemblies, in particular in the diagnosis and treatment of disease.

### BACKGROUND OF THE INVENTION

In biologic systems, organic molecules exhibit a remarkable level of control over the nucleation and mineral phase of inorganic materials, such as calcium carbonate and silica, and over the assembly of crystallites and other nanoscale building blocks into complex structures required for biologic function. This control could, in theory, be applied to materials with interesting magnetic, electrical, or optical properties.

Materials produced by biologic processes are typically soft, and consist of a surprisingly simple collection of molecular building blocks (i.e., lipids, peptides, and nucleic acids) arranged in astoundingly complex architectures. Unlike the semiconductor industry, which relies on a serial lithographic processing approach for constructing the smallest features on an integrated circuit, living organisms execute their architectural “blueprints” using both covalent and non-covalent forces acting simultaneously upon many molecular components. Furthermore, these structures can often elegantly rearrange between two or more usable forms without changing any of the molecular constituents.

The synthesis of single crystal nanowires using phage as a template for the assembly of regular nanostructures to be used in the semiconductor industry has been reported (Mao et al., 2004). Mao et al. report that the nanostructural assembly is mediated by an interaction between peptides inserted into the major capsid protein and the nanoparticles; thus, their methodology requires selection and insertion of peptides into the phage structure (Whaley et al., 2000; Mao et al. 2003; Lee et al., 2002). However, Mao et al. (2004) have not shown compelling evidence that nanoparticles do not assemble onto phage when the pVIII gene has not been modified. This is important because, in nature, self-assembly or direct-assembly of molecules and particles is often directed by non-specific hydrophobic, van der Walls, and/or electrostatic interactions (Dutta and Hofmann, 2003).

The use of biologic materials to produce medicinal reagents with desirable optical and magnetic properties provides a possible solution to resolving limitations of traditional diagnostic and therapeutic methods. One factor in this approach is identifying the appropriate combination of components and process conditions for creating unique and specific combinations of agents to aid in the detection and treatment of various biological disorders.

### BRIEF SUMMARY OF THE INVENTION

Embodiments of the invention include a bacteriophage (phage-nanoparticle) assembly comprising a filamentous bacteriophage forming a bacteriophage scaffold associated with a plurality of conductive nanoparticles that form a bacteriophage assembly. The filamentous bacteriophage may include, but are not limited to fd, f1, or M13 bacteriophage. In particular aspects the bacteriophage is a fd bacteriophage. The assembly may further comprise a targeting moiety (a peptide or protein displayed on the bacteriophage or operatively coupled to the bacteriophage). The targeting moiety may be operably coupled (which includes being displayed on the surface of a bacteriophage) to a bacteriophage, a conductive cluster, or a bacteriophage assembly. In a preferred embodiment, the targeting moiety is a peptide and in a more preferred embodiment the peptide is a cyclic peptide. Typically cyclic peptide is a CX₇C peptide, wherein C is cysteine and X is a random amino acid. In certain embodiments, larger protein domains such as antibodies or single-chain antibodies can also be displayed on or operativley coupled to the surface of phage particles, i.e., a targeting moiety (Arap et al., 1998a). The assembly may also comprise a targeting moiety operably coupled, in particular covalently coupled, to a component of the assembly, e.g., phage or nanoparticle. In one aspect the targeting moiety is a peptide. The targeting peptide can be selected from the group consisting of SEQ ID NO:1 to SEQ ID NO:685. In particular aspects of the invention the targeting moiety is comprised in a pIII or pVIII protein of the bacteriophage. In further aspects, targeting moieties may be identified by screening peptides presented or included in the pIII and/or pVIII protein, in preferred embodiments the pVIII protein. A conductive nanoparticle of the invention is typically a metallic conductive nanoparticle. In certain aspects the metallic conductive nanoparticle comprises Au, Ag, Pt, Ti, Al, Si, Ge, Cu, Cr, W, Fe, or a corresponding oxide. In a still another aspect the conductive nanoparticle is a Au cluster. A conductive nanoparticle may be 2, 25, 50. 75, 100 to75, 100, 150, 200, 250, 280, 400, 450, 500 nm, or 2 to 250 nm, or 50 to 200 nm, or 75 to 150 nm in diameter, including all values and ranges therebetween.

An assembly of the invention may further comprise an organizing agent that promotes organized packing of conductive nanoparticles. An organizing agent may include, but is not limited to a peptide, a pyrrole, an imidazole, histidine, cysteine, or tryptophan. Phage-nanoparticle assemblies may comprise a therapeutic agent, such as a therapeutic molecule of nucleic acid. An organizing agent may induce aggregation, or couples two or more particles to form assemblies of the invention and is not limited to agents that induce an orderly arrangement of molecules, such as a lattice. In certain aspects, the therapeutic agent is an organizing agent. The phage-nanoparticle assembly of the invention may be comprised in a pharmaceutically acceptable composition. Other embodiments of the invention include a cell comprising or operativley coupled to a bacteriophage (phage-nanoparticle) assembly of the invention.

Still other embodiments of the invention include methods of producing a bacteriophage assembly comprising the steps of: a) providing a first filamentous bacteriophage solution having a bacteriophage concentration of between 10², 10³, 10⁴ to 10⁵, 10⁶, 10⁷, 10⁸, 10⁹, 10¹⁰, 10¹¹, 10¹² transduction units (TU) (including any value or range therebetween) per microliter; b) providing a second solution comprising conductive atomic or molecular clusters having a diameter in the range of 2, 20, 50, 100, 200, 280, 400 nm to 500, 600, 700, 800, 900, 1,000 nm (including all values or ranges therebetween, the solution having an absorption of 1.2 to 1.5 absorbance units at a wavelength appropriate for the conductive cluster; c) contacting the bacteriophage solution with a solution of conductive clusters under conditions in which the conductive clusters associate with the bacteriophage forming a bacteriophage assembly; and d) isolating the bacteriophage assembly. The method may further comprise providing a series of bacteriophage solutions comprising a dilution series of bacteriophage, wherein each solution of the series is mixed individually with a solution of conductive clusters. A bacteriophage dilution series may comprise successive 1 to 2 dilutions of the first bacteriophage solution. The mixed solutions may be combined in order from the mixture with the lowest concentration of bacteriophage to the mixture with the highest concentration of bacteriophage. However, a particular concentration(s) of bacteriophage solution may also be used in practice of the invention. A bacteriophage solution will typically contain 10⁵, 10⁶, 10⁷, 10⁸, 10⁹, 10¹⁰, 10¹¹, 10¹² TU per microliter or concentrations there between. In certain aspects the bacteriophage solution will be 10⁹ TU per microliter.

In still other aspects of the invention conductive clusters may be 2 to 500 nm in diameter, 2 to 250 nm in diameter, 50 to 200 nm in diameter, 75 to 150 nm in diameter. In certain aspects of the invention the bacteriophage solution, the conductive cluster solution, or both the bacteriophage solution and the conductive cluster solution contains an organizing agent that increases the ratio of conductive clusters to bacteriophage in the assembly. The organizing agent is preferably imidazole. Imidazole concentrations may be in the range of 0.1 μM to 2 M, 0.5 μM to 1.5 M or various ranges therebetween. In a preferred embodiment the imidazole concentration is 1 M. In further aspects of the invention the bacteriophage solution, the conductive cluster solution, or both solutions comprise an acetate, carbonate, bi-carbonate, chloride, cyanide, nitrate, nitrite, phosphate, sulfate, citrate, or borate salt. The salt concentration may be 0.05 mM, 1 mM, 5 mM, 10 mM, 20 mM, 28 mM, 40 mM, 50 mM, 100 mM, 200 mM, 280 mM, 400 mM, 500 mM, or 1 M.

In yet further embodiments of the invention a detection method may comprise a) contacting a cell with a bacteriophage assembly of the invention that selectively binds a cell forming a cell/bacteriophage assembly complex; b) exposing the cell/bacteriophage assembly complex to a radiation source; and c) detecting a signal produced by the cell associated bacteriophage assembly. The radiation source may be an infrared radiation source. A cell may be comprised in a tissue, an organ, or an organism. In a preferred embodiment the cell is comprised in a tissue sample. The tissue sample may be a biopsy or a fluid sample. In certain aspects of the invention the fluid sample is analyzed by flow assisted cell sorting. Typically, a cell is sorted by the presence or absence of a detectable signal. A detectable signal can be, but is not limited to a Raman, enhanced fluorescence, absorption, elastic scattering signal, or other optical signal.

Embodiments of the invention also include methods of screening peptides for use in nanoparticle assembly comprising a) providing a phage display library; b) contacting the phage display library with metal nanoparticles; c) assessing the formation of a phage-nanoparticle assembly. Assessing a signal may comprise detecting an optical property of the assembly. An optical property may include, but is not limited to SERS, Raman, fluorescence, or color detection.

A further embodiment of the invention may include a cell ablation method comprising a) contacting a cell with a bacteriophage assembly of invention that selectively binds the cell forming a cell/bacteriophage assembly complex; and b) exposing the cell/bacteriophage assembly complex to electromagnetic radiation, wherein the cell associated bacteriophage assembly is heated and the cell is incapacitated. In certain aspects the cell is killed. Electromagnetic radiation includes, but is not limited to near infrared light. The cell may be comprised in a tissue, an organ, or an organism.

Yet further embodiments of the invention include methods of producing a bacteriophage assembly by remote assembly of conductive clusters comprising a) contacting a cell with a filamentous bacteriophage forming a bacteriophage/cell complex; and b) contacting the cell/bacteriophage complex with a plurality of conductive clusters, wherein a cell/bacteriophage assembly complex is formed. The cell may be affixed to a slide, comprised in a tissue, an organ, or an organism.

Still further embodiments of the invention include kits comprising a filamentous bacteriophage and a conductive atomic or molecular clusters having a diameter of 2 nm to 1,000 nm, and various sizes there between, disposed in a suitable containers. The kit may further comprise an organizing agent for inducing closer packing of the conductive clusters and increasing the conductive cluster to bacteriophage ratio in a bacteriophage assembly.

Embodiments of the invention also include devices for the detection of bacteriophage assemblies comprising a Raman detector operatively coupled to a cell sorter. In a preferred embodiment the cell sorter is a microfluidic device.

In other embodiments, phage-nanoparticles of the invention can be used as standalone nanosensor to detect molecules and/or cells that interact with phage and/or other molecules within the Au-phage assembly. This can be accomplished using fluorescence, absorption, elastic scattering and Raman detection.

In other aspects, phage libraries may be modified through the pVIII protein to select peptides according to their nanoparticle affinity which would be applicable to different applications based on the thermodynamics of the interaction. For example, high affinity peptides would be more applicable to remote assembly of nanoparticles at a binding site; or lower affinity could be desirable for drug delivery when disassembly of the phage-nanoparticle is desirable.

In still further embodiments, phage-nanoparticle assemblies of the invention may be used to screen for peptides which induce specific/distinct optical and physical properties when interacting with nanoparticles within a phage-assembly, such as peptide specific SERS spectrum signatures which can be use as Raman labels; peptide specific fluorescent spectrum signatures which can be use as fluorescent labels; as well as other non-linear optical properties. Other properties that may be screened for includes, but is not limited to peptide specific color of phage-nanoparticle solutions; electron conductivity (conducting nano-assemblies); and peptide specific fractal dimension within Au-phage assembly to name a few.

In other embodiments, the phage nanoparticle assemblies may be employed for delivery of phage, nucleic acids, nanoparticles and/or other therapeutic agents. In particular aspects, phage nanoparticle assemblies may be used to deliver gene therapy applications.

As used herein in the specification, “a” or “an” may mean one or more. As used herein in the claim(s), in conjunction with the word “comprising,” the words “a” or “an” may mean one or more than one. As used herein “another” may mean at least a second or more of an item.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1E (FIG. 1A) Network of Au-phage assemblies as a function of phage input in aqueous medium: (left to right) 2.0×10⁹ TU/ml, 1.0×10⁸ TU/ml, 0.5×10⁷ TU/ml, 0.2×10⁷ TU/ml and 0.1×10⁶ TU/ml. (FIG. 1B) TEM of Au nanoparticles and phage complex (scale bar: 500 nm); arrows indicate Au-phage complex. (FIG. 1C) Bacterial infection with purified Au-phage assembly (centrifugation purified). TU (transducing units or number of colonies) shown for phage present in purified Au-phage pellet and supernatant. (FIG. 1D) Extinction spectra of Au-phage solutions in PBS (pH 7.4) under increasing phage input: 4×10⁸ TU/ml, 4×10⁷ TU/ml, no phage and 4×10⁷ TU/ml in water (no PBS). (FIG. 1E) SERS spectrum of Au-phage in water using 785 nm incident laser light (10 sec. integration); inset shows full SERS spectrum.

FIG. 2. Phage binding and internalization in cultured cells. Cell monolayers were incubated with different phage preparations in MEM containing 2% FBS, as indicated; Au—RGD-4C scaffolds (1×10⁷ TU), Au-Fd-tet (1×10⁷ TU, Au-phage negative control), Fd-tet phage (1×10⁹ TU, negative control), (right), and RGD-4C displaying phage (1×10⁹ TU, positive control). Phase contrast is shown for the fields evaluated by Cy3 fluorescence. KS cells were seeded in 10% FBS in MEM (glass slide chamber) and allowed to attach for 12 hours, and exposed to each phage preparation. After 12 hours incubation at 37° C., cells were washed with PBS, glycine/NaCl (pH 2.8, to remove weakly bound phage), and fixed with 4% paraformaldehyde. Next, cells were permeabilized using 0.2% Triton X-100 for 5 min. Immuno fluorescence staining to visualize the cell bound phage was performed by using a rabbit anti-phage as primary antibody and Cy3 anti-rabbit IgG as the secondary antibody. Cy3 dye fluorescence is shown for the test samples and controls. The images were acquired with an Olympus fluorescence microscope equipped with a Hg lamp and a band pass excitation filter (528 nm to 555 nm) in the fluorescence excitation path, and in the emission path had a long pass dichroic filter along with a long pass filter (560 nm to 590 nm).

FIG. 3. Targeting of lung endothelial cells with Au-phage. Lung targeting phage alone internalizes into lung endothelial cells (left). Au—CGSPGWVRC (SEQ ID NO:1)-Imid scaffolds localize to the cell surface (middle). The signal enhancement is observed with the Au—PGWV (SEQ ID NO:2)-phage-Imid, but not with phage alone. All measurements were carried with the same settings. A 543 nm laser (excitation) and 560-590 nm (emission) was used for the detection of Cy3.

FIG. 4. Immunofluorescence of the lung after i.v. injection of Au—CGSPGWVRC (SEQ ID NO:1)-Imid (right panel). No signal was observed in the control (left panel) and minimal background was observed with fd-Au-Imid (middle panel). All images were captured under identical conditions.

FIG. 5. Cell suspension detection scheme using SERS including spectrometer and fiber probe schematics (left). SERS spectra of cells in solution, after incubation with Au—RGD-4C scaffold, Au-Fd-tet, RGD-4C phage. KS cell monolayers were exposed to phage, after targeted internalization and washes, cell-Au-phage complexes were scraped and resuspended in PBS. The samples were transferred to a glass cuvette and the SERS signal was determined with a spectrometer (R2001, Raman Systems).

FIG. 6. Schematic of non-invasive SERS detection across mouse skin of intra-tissue injected Au-imidazole clusters. SERS spectra description: the curve (in vivo) is the spectrum generated by the Au-Imidazole clusters inside the target tissue; curve (in vivo) is the spectrum from tissue prior to Au-imidazole cluster injections; and in-vitro is the spectrum from Au-imidazole clusters in solution measured in a glass cuvette.

FIG. 7. SERS spectra of KS cell suspended solution, which were incubated with Au—RGD-4C-imid scaffold, Au-Fd-tet-Imid, or RGD-4C phage. Spectrum of Au—RGD-4C-imid solution before added to cells. KS cells grew on a Petri dish to a monolayer coverage; second internalizing Au-phage-imidazole (and controls on separate dishes); third washing the cells with glycine to remove loosely bound phage; next, carefully scraped the cells from the petri dishes (counted the cells and normalized all cell concentration with PBS; and last the cells were resuspended in PBS and measured the SERS signal of the suspended cells in a glass cuvette.

FIG. 8. In vivo targeting of melanoma using Au-phage nanoshuttles. First three columns are fluorescence images of mouse frozen tissue sections: first column cy3 fluorescence, second FITC and third is the merge of the first two. Fourth column is brightfield image of respective areas in the first three columns.

FIG. 9. Magnetic response of Au-phage-im hydrogels prepared with different Au sizes (1×10⁸ TU). Each row show pictures of the same well under varying magnetic field position. Arrows indicate direction of movement

FIG. 10. Magnetic response of Au-phage-im hydrogels prepared with different Au sizes (0.5×10⁸ TU). Each row show pictures of the same well under varying magnetic field position. Arrows indicate direction of movement

FIG. 11. Magnetic response of Au-phage-im hydrogels prepared with both phage inputs. Each row show pictures of the same well under varying magnetic field position. Arrows indicate direction of movement

FIG. 12. Magnetic response of Au-phage-im hydrogels prepared with both phage inputs. Each row show pictures of the same well under varying magnetic field position. Arrow indicate direction of movement.

FIGS. 13A and 13B. (FIG. 13A) Gold agglomeration induced by 0.25M NaCl. (FIG. 13B) Formation of phage networks as a function of pH.

FIGS. 14A and 14B. (FIG. 14A) Hydrogel Formation. (FIG. 14B) Plot of extinction at λmax and pVIII Charge vs. pH. Relative charge from theoretical calculation based on individual pVIII amino acid—not taking into account peptide structure (scripps.edu/cgi-bin/cdputnam/protcalc)

FIGS. 15A and 15B. (FIG. 15A) Hydrogel Formation. (FIG. 15B) Surface Plasmon Red Shift and pVIII Charge vs. pH. Relative Charge from theoretical calculation based on individual pVIII amino acid—not taking into account peptide structure (scripps.edu/cgi-bin/cdputnam/protcalc).

FIG. 16. Hydrogel Formation: Extinction at 270 nm and pVIII Charge vs. pH.

FIG. 17. Western Blot from NSC Cultured within Au-phage Hydrogels.

FIGS. 18A and 18B. Au—RGD-4C-GFP Hydrogel HEC293 Cells Cultured within Hydrogel Structure (4 days) (FIG. 18A) real color and (FIG. 18B) GFP fluorescence merged.

FIG. 19. HEC293 Cells Cultured in Au-phage-GFP Hydrogel for 10 Days. Shows RGD-4C and Fd-tet as brightfield and as GFP fluorescence.

FIG. 20. T2 Average from MRI Fat Images.

FIGS. 21A-21F. (FIG. 21A) Strategy for Au assembly onto phage nanoparticles. Imidazole and the spheres (Au nanoparticles; not drawn to scale; the gold particles have a diameter of 44±9 nm, and the pVIII capsid peptide a thickness of ˜6 nm). (FIG. 21B) Vials of nanoparticle solutions: Au-phage hydrogel (left) and suspension of purified Au-phage-imid (right; suspended from hydrogels precursor). (FIGS. 21C-21D) Hydrogel formed with RGD-4C displaying phage (scale bar, 20 μm); (FIG. 21C) C17.2 murine neural stem cells cultured within hydrogel for 24 h. Cell accumulation followed by cell induced network displacement (FIG. 21C—arrows point to cells within the network); (FIG. 21D) control hydrogel (no cells). (FIG. 21E) TEM of purified networks (scale bar 500 nm; inset scale bar 100 nm): Au-phage (top) and Au-phage-imid (bottom). (FIG. 21F) Bacterial infection with purified Au-phage (top) and Au-phage-imid (bottom) networks; T.U. shown for purified and functional Au-phage and Au-phage-imid solution and for unbound phage present in the supernatant from centrifuged network solutions.

FIGS. 22A-22C. (FIG. 22A) Light absorption spectrum at various phage input (indicated in the legend) in the presence of 0.25 M NaCl (no phage, bottom curve). (FIG. 22B) Light extinction at 710 nm for Au-phage solutions as a function of phage input at various pH (10 mM boric acid, pH 5.2; 10 mM Na borate buffer, pH 6.5; 10 mM Na borate, pH 9.2; or 10 mM NaOH, pH 14.0). (FIG. 22C) Cartoon illustrating electrostatic interaction of Au (yellow spheres) with phage (elongated structures; not to scale). Arrows point to pVIII major capsid protein (pVIII) and pill minor capsid protein (pIII).

FIG. 23A-23C. (FIG. 23A) Light absorption spectrum of purified and suspended Au-phage-imid (dark blue), Au-phage (red). (FIG. 23B) Temperature as a function of illumination (785 nm laser light) time of Au-phage-imid and Au-phage solutions; the controls (open circles) are the solutions of Au, Au-imid and phage. The concentrations of all solutions carrying Au were normalized according to the area under the absorption region of the spectra (above 475 nm). The solution temperature was measured with a digital temperature probe (Teflon coated cables of type-K beaded sensor coupled to a Fisherbrand Traceable Double Thermometer with Computer Output from Fisher Scientific) immersed in 280 μl of solution and 5 mm away from the laser focal point. (FIG. 23C) SERS of Au-phage-imid and Au-phage measured in water.

FIG. 24. Confocal fluorescence. KS1767 cells incubated with phage preparations (input of 1.0×10⁷ T.U.) and labeled with anti-fd bacteriophage antibody (first column), SYTOX green nucleic acid stain (second column) and an anti-β1 integrin antibody demarking the cell surface (third column); fourth column shows merged images (scale bar, 10 μm). RGD-4C phage, Au—RGD-4C networks and Au—RGD-4C-imid networks.

FIGS. 25A and 25B. Darkfield images of cell bound Au-phage networks using light from a microscope mercury lamp. Confluent KS 1767 cells incubated with phage preparations (input of 1.0×10⁷ T.U.): (FIG. 25A) Au—RGD-4C and (FIG. 25B) Au-fd-tet (control insertless phage) networks.

### DETAILED DESCRIPTION OF THE INVENTION

For the reasons stated above, and others, the Inventors describe additional compositions, and related methods and devices, for the use of phage-nanoparticle assemblies. Embodiments of the invention include compositions, methods and devices related to phage-nanoparticle assemblies and their use in a variety of methods including detection methods, in vitro and in vivo diagnostic methods, direct and/or indirect therapeutic methods, or combinations thereof. A “nanoparticle” refers to a conductive atomic or molecular cluster, such as a metal cluster, having a diameter on the nanometer scale and may be as small as a cluster of atoms (approximately 5 angstroms) and as large as or larger than 1,000 nm in diameter. Phage-nanoparticle assemblies of the invention comprise a plurality of nanoparticles complexed with one or more phage particles to form a phage-nanoparticle assembly. In certain aspects, the phage-nanoparticle assembly may also include other agents, including but not limited to organizing agents and/or therapeutic agents. Organizing agents may also be, but need not be a therapeutic agent. Further embodiments of the invention may include devices designed or adapted for use with phage-nanoparticle assemblies. Synonomous with the term nanoparticle is the term network or fractal network of Au and phage nanoparticles. The nanoparticle assemblies have been shown to possess fractal characteristics, which may be relevant electrostatic signal enhancing mechanism of the fractal structures of metal nanoparticles.

Various aspects of the invention use a phage-nanoparticle assembly as a probe, that may or may not comprise targeting entities; with various detection devices and/or methods including, but not limited to instrumentation and detection schemes allowing elastic (angle dependent light scattering, ADLS), inelastic (Raman) scattering detection, surface enhanced Raman scattering (SERS); enhanced fluorescence detection; and/or absorption of near infrared (NIR) electromagnetic radiation.

Given the challenges for reproducibly building at the nanometer scale, the assembly of phage and nanoparticles is an ideal system to control the fabrication and application of biological assisted nanoparticle-assemblies. In certain embodiments, metal nanoparticles are preferred with Au or Ag nanoparticles being more preferred. Certain advantages of the invention include streamlined and “bottom-up” approaches for assembling nanoparticle architectures. A bottom up approach refers to the combination of particles or sub-elements to produce a more complex structure, as oppose to a top down approach that typically includes the removal of particles, sub-elements, or material to produce a product. The present application is based on the premise that, in nature, the self-assembly or direct-assembly of molecules and particles is often directed by non-specific hydrophobic, van der Walls, and/or electrostatic interactions (Dutta and Hofmann, 2003). The assembly of phage and nanoparticles (e.g., Au nanoparticles) may also occur spontaneously through similar interactions. The present application describes the design and validation of a method for phage-based nanoparticle-assembly, preferably Au-phage based nanoparticle-assembly, without genetic manipulation of proteins that interact with the nanoparticles or complex conjugation chemistry.

Embodiments of the invention provide a simple and efficient coupling procedure for Au-phage coupling procedure. Certain aspects may or may not include genetic engineering, complex chemical conjugation, need for synthetic peptides (synthetic peptides are costly with possible solubility and there may be some problems with conjugation).

The compositions of the present invention, in certain aspects, provides ideal chemical/physical properties of phage and gold nanoparticles, such as phage, which are programmable tissue targeting, physical and chemical robust, synthesized by bacteria, and are nonpathogenic to humans; Gold, which is relatively inert and non-toxic, inexpensive, reproducibility and ease of manufacturing procedure, and methods and compositions that provide desirable physical and chemical property of tunability, which includes NIR Surface plasmon absorption by changing solution conditions, incorporation of additional molecules or Nanoparticles (SERS, tunable photon-to-heat conversion, magnetic response (MRI); Adhesive or non-adhesive properties; or Drug and gene carrier).

Potential Applications include, but are not limited to cell matrix for stem cell growth and differentiation, tissue engineering, magnetic storage: bio-inorganic hardrive; microfluidic: NIR light or magnetic response; bio-electronics: cell circuitry; energy storage: bio-inorganic battery by coupling a secondary nanoparticle; electrochemistry: bio-inorganic electrodes for bio and inorganic detection; and optics and lasers: second harmonic generation and SERS.

In still further embodiments, an Au-phage-Iron oxide can be used for phage detection and isolation from in vivo or in vitro screening. For example, mice can be injected with the iron oxide and/or the nanoshuttle. Organs or samples of organs may then be isolated, ground followed by separation of Au-phage-FeO nanoshuttles using a magnet. The various detection schemes described herein and known to those skilled in the are can be used to detect the nanoshuttles (i.e., bacteriophage scaffolds or assemblies).

Aspects of the invention include, but are not limited to methods and compositions were: 1) in one aspect there is no genetic manipulation of phage pVIII capsid; if genetic manipulation is performed, peptides are identified by methods other than pIII phage display libraries; 2) in another aspect nanoparticles assembly can be accomplished using non-genetic manipulation, e.g., using conjugation chemistry; 3) in still another aspect there is no manipulation of the capsid, only manipulation of solution conditions; 4) in yet still another aspect is to screen material binding peptides using phage display libraries where peptides are displayed on pVIII, and not pIII. The location of the peptides, in some instances effect the the screening procedure; and 5) certain aspects use a combination of different sizes (separately and together) to form the nanoshuttles using a wide range of metal, non-metal nanoparticles and molecules.

### I. Phage-Nanoparticle Assemblies

By interfacing biotechnology and nanotechnology, the inventors are generating innovative bio-medical materials and applications, and applying such towards, for example, diagnosing and treating various disease states. In light of the need for additional approaches for the fabrication of nanostructures, the proposed assembly methods and the applications of phage-nanoparticle assemblies are of significance. Embodiments of the invention combine phage technology (e.g., phage display or BRASIL) and nanoparticles (e.g., gold (Au) nanoparticles) in the molecular assembly or self-assembly or direct assembly of nanoparticles and phage into a phage-nanoparticle assembly or scaffold structures.

Generally, there are two approaches for building nanoassemblies (Dutta and Hofmann, 2003; Seeman and Belcher, 2002): one, the “top-down” approach, where nano-fabrication starts at the macroscopic scale from bulk materials that are manipulated, usually through etching, deposition, milling and/or polishing, into nanoscale features. The poor efficiency and low yields of the “top-down” process often makes such an approach impractical. On the other hand, the “bottom-up” approach starts at the nanometer scale by assembling atoms and molecules into molecular composite structures, providing great structural and chemical diversity (Dutta and Hofmann, 2003). A major challenge for the “bottom-up” approach is the difficulty of manipulating and controlling the assembly at the molecular level. Biological systems offer an appealing opportunity for molecular engineering because they are driven by biochemical organization into complex assemblies that are the most energetically favorable. Moreover, pattern formation in nature occurs along with molecular selectivity and recognition (Seeman and Belcher, 2002). Phage-assisted assembly of nanoparticles is therefore ideal for fabricating functional bio-molecular nanostructures, which in essence, capture biological, chemical, and physical properties that may meet diagnostic and therapeutic needs.

Lee et al. (2002) were the first to demonstrate the synthesis of inorganic nanoparticles onto phage particles. However, their method requires the selection and insertion of nucleating peptides into the phage structure through genetic manipulation (Mao et al., 2003; 2004). The display of certain peptides within the virus major capsid may result in low yields or even failure of phage production (Barbas et al., 2001).

Typically, filamentous phage (M13, fd, f1) have a filamentous capsid with a circular ssDNA molecule. The genome typically contains 10 genes but none for a lysis protein. Virions are enveloped. The filamentous phage, typically, only infect E. coli cells carrying the F plasmid since the phage must adsorb to the F pilus to gain entry to the cells. Their life-cycle involves a dsDNA intermediate replicative form within the cell which is converted to a ssDNA molecule prior to encapsidation. This conversion is the major reason for the great utility of the phage as a molecular biological laboratory tool: they provide an easy means to prepare ssDNA for DNA sequencing. The best known example is bacteriophage M13 which has been adapted for use as a cloning and sequencing vector. The wild-type M13 genome is 6407 by in length; the modified cloning vector is 7249 by in length. Other relatives of M13 are fd and f1.

The present invention demonstrates both, the synthesis of biologically functional phage-nanoparticle assemblies and self-assemblies with or without genetic manipulation and typically do not require a recombinant phage peptide for nucleation of the particles. Phage-nanoparticle self-assembly or direct-assembly of the present invention may be induced and controlled without genetic engineering or complex conjugation chemistry. This streamlined approach enables self-assembly or direct-assembly of biologically based nanoparticles.

Filamentous phage (f1, fd, M13 and the like) are single-stranded DNA viruses encapsulated in a long protein cylinder capsid. The Fd phage do not kill their bacterial host and therefore, infected-bacterial cultures can produce up to 10¹² phage particles per ml of culture, which in turn may be further concentrated. Phage particles are extruded by the bacterial membrane/cell wall into the media and phage particles can be easily recovered by, for example, a two-step protocol including: (i) centrifugation of the culture to separate phage from bacteria; (ii) precipitation of the phage in the supernatant, e.g., by the addition of PEG and NaCl. Further purification can be obtained by CsCl gradients. Filamentous phage are notorious for their resistance to extreme conditions, such as high salt conditions, acidic pH, chaotropic agents (urea 4M), and prolonged storage (several years at 4° C. without significant loss in viability). Very few other classes of viruses would survive such conditions, which is one characteristic that makes Fd phage a preferred tool of the present invention (Barrow and Soothill, 1997).

The Inventors have devised and implemented a “bottom-up” approach for the directed assembly of nanoparticles and phage, establishing conditions that are more optimal for phage-nanoparticle assembly. Phage-nanoparticle assemblies have been reproducibly synthesized through self-assembly direct-assembly of nanoparticles onto phage templates by the manipulation of solution properties. Morphologically stable and distinct networks of self-assembled, biologically active phage-nanoparticle assemblies have been generated. Moreover, varying conditions, such as phage and/or nanoparticle concentration, and/or the presence and concentration of salts, can alter the mechanical and optical properties of the phage-nanoparticle assembly. These results not only support the assembly of nanoparticles onto phage without covalent bonding, but also strongly suggest the electrostatic nature of the Phage-nanoparticle interaction (Shipway et al., 2000).

The inventors have found that the surface plasmon (SP) absorption wavelengths of the phage-nanoparticle assembly can be tuned by changing the phage input in the presence of salts. Phage input includes, but is not limited to 10¹, 10², 10³, 10⁴, 10⁵, 10⁶, 10⁷, 10⁸, 10⁹, 10¹⁰, 10¹¹, 10¹², 10¹³, or 10¹⁴ TU/ml or any range or value therebetween. A solution in which assembly can occur may have a salt concentration of 0.05 mM, 1 mM, 5 mM, 10 mM, 20 mM, 28 mM, 40 mM, 50 mM, 100 mM, 200 mM, 280 mM, 400 mM, 500 mM, 1 M, 2 M, 3 M, 4 M, 5 M salt. Salts may include, but are not limited to acetates, carbonates, bi-carbonates, chlorides, cyanides, nitrates, nitrites, phosphates, sulfates, citrates, borates, and the like. Exemplary nanoparticle compositions may include Au, Ag, Pt, Ti, Al, Si, Ge, Cu, Cr, W, Fe, and their corresponding oxides, preferably Au or Ag. In addition, group III-V and II-VI semiconductors, such as CdSe, CdS, CdTe, and GaAs, can be used to prepare nanoparticles, see for example U.S. Patent application 20040077844, which is incorporated in its entirety herein by reference.

Typically, a shift in the SP absorption to longer wavelengths indicates particle assembly. A higher phage input stabilizes phage-nanoparticle assemblies by preventing salt-induced aggregation. Without phage present nanoparticles may aggregate and precipitate. The nanoparticle interaction with the phage surface may be characterized using surface enhanced Raman scattering (SERS) spectroscopy (Kniepp et al., 1999), as well as other methods known in the art.

A. Phage Assemblies Comprising Other Agents

In certain embodiments, phage-nanoparticle assemblies may include an organizing agent (e.g., imidazole), a therapeutic agent, and/or other agents. Other agents may be supplied before during and/or after assembly process. These structures may be optimized for biological detection, therapy, or for other beneficial characteristics and properties. The assembly of phage-nanoparticles begins with separate mixtures of nanoparticles and phage. The mixtures are typically pooled together, incubated for a time during which the phage and nanoparticles assemble, and the resulting assemblies purified. In certain aspects, an organizing agent, such as imidazole, may be incorporated into the phage solution, the nanoparticle solution, both the phage and the nanoparticle solutions, or other solutions or mixtures used in the process at 0.1, 0.2, 0.3, 0.4, 0.5, 0.6, 0.7, 0.8, 0.9, 1, 1.2, 1.4, 1.6, 1.8, or 2 M, including any value or range therebetween. The organizing agent may modify, induce, enhance or accelerate aggregation and packing of nanoparticles. In preferred embodiments, the nanoparticles are gold (Au) nanoparticles. Typically, the nanoparticles and phage alone assemble into loose nanoparticle assemblies and the addition of an organizing agent (e.g., imidazole) produces a denser structure resulting from organizing agent-induced nanoparticle aggregation. The difference in structure gives rise to the distinct optical and physical properties between the phage-nanoparticle and phage-organizing agent-nanoparticle assemblies. The more packed (organized) phage-nanoparticle shows a shift in their surface plasmon (SP) absorption wavelength from the visible into the near infrared (NIR) region of the electromagnetic spectrum (700 nm to 900 nm).

The shift in SP, which is typically a result of the interaction between the nanoparticles, allows the phage-nanoparticle assembly to absorb the near infrared (NIR) incident light and then covert the energy of the absorbed photons into heat. The larger absorption wavelength shift for the phage-organizing agent-nanoparticle assemblies, which may overlap with the wavelength of the incident laser light (e.g., 785 nm), provides for a more efficient conversion of the incident NIR laser light into heat than phage-nanoparticle assemblies without an organizing agent (as evidenced by larger temperature change). Interestingly, the shift in plasmon resonance of an Au-phage (without imidazole) was small, yet significant. This energy conversion process led to the adaptation of such particles for tissue photo-ablation applications.

One can modulate the cell targeting properties of the phage-assemblies by manipulating the assembly structure with the inclusion of additional agents, e.g., imidazole. Not only may the structural differences alter localization and other physical properties, but a nanoparticle may be coupled to a targeting agent that may alter the localization of a phage-nanoparticle assembly. The incorporation of an organizing agent suggests that molecules with therapeutic properties could be assembled within the phage-nanoparticle assembly for the purpose of delivery of a therapeutic molecule.

B. Targeted and Non-Targeted Phage-Nanoparticle Assemblies

The integration of phage based targeting and/or peptide-based targeting with nanotechnology provides for the targeting or localization of phage-nanoparticle assemblies. By incorporating phage particles in the assemblies or scaffolds, the Inventors have generated an improved modular targeted delivery system that can be applied to any tissue of interest. Phage are by definition designed for receptor mediated tissue targeting. Targeted phage can be selected based on their cell binding and internalization properties. Exemplary studies have been developed based on characterized ligand-receptor pairs selectively accessible upon intravenous administration, however a variety of phage or peptides selected by methods known in the art for preferential binding to a cell type, tissue, organ and/or organism may be used. In certain embodiments, a phage may be selected by using methods known in the art, exemplified in U.S. Patent applications 20028152578 and 20040048243, each of which are incorporated herein by reference.

The methods for identification of targeting moieties may involve the administration of phage display libraries. Various methods of phage display and methods for producing diverse populations of peptides are well known in the art. For example, U.S. Pat. Nos. 5,223,409; 5,622,699 and 6,068,829, each of which is incorporated herein by reference, describe methods for preparing a phage library. The phage display technique involves genetically manipulating bacteriophage so that small peptides can be expressed on their surface (Smith et al., 1985, 1993). The potential range of applications for this technique is quite broad, and the past decade has seen considerable progress in the construction of phage-displayed peptide libraries and in the development of screening methods in which the libraries are used to isolate peptide ligands. For example, the use of peptide libraries has made it possible to characterize interacting sites and receptor-ligand binding motifs within many proteins, such as antibodies involved in inflammatory reactions or integrins that mediate cellular adherence. This method has also been used to identify novel peptide ligands that serve as leads to the development of peptidomimetic drugs or imaging agents (Arap et al., 1998a). In addition to peptides, larger protein domains such as antibodies or single-chain antibodies can also be displayed on the surface of phage particles (Arap et al., 1998a).

Selection of phage for use in the invention preferentially employ libraries of random or non-random peptides or polypeptides expressed as fusion proteins with the gene III capsule protein in the fUSE5 vector (Pasqualini and Ruoslahti, 1996). The number and diversity of individual clones present in a given library is a significant factor for the success of selection. Primary libraries are preferred, which are less likely to have an over-representation of defective phage clones (Koivunen et al., 1999). The preparation of a library may be optimized to between 10⁸-10⁹ transducing units (T.U.)/ml. A bulk amplification strategy may be applied between rounds of selection.

Phage libraries may display non-cyclic, cyclic, or double cyclic peptides or polypeptides (such as antibodies). Phage libraries displaying 3 to 10 random residues in a cyclic insert (CX₃₋₁₀C) are preferred, since single cyclic peptides tend to have a higher affinity for the target organ than linear peptides. Libraries displaying double-cyclic peptides (such as CX₃C X₃C X₃C; Rojotte et al., 1998) have been successfully used. However, the production of the cognate synthetic peptides, although possible, can be complex due to the multiple conformers with different disulfide bridge arrangements.

The term “antibody” is used to refer to any antibody like molecule that has an antigen binding region, and includes antibody fragments such as Fab′, Fab, F(ab′)2, single domain antibodies (DABs), Fv, scFv (single chain Fv), and the like. Techniques for preparing and using various antibody based constructs and fragments are well known in the art. Means for preparing and characterizing antibodies are also well known in the art (See, e.g., Antibodies: A Laboratory Manual, Cold Spring Harbor Laboratory, 1988; incorporated herein by reference). Examples of phage display methods that can be used in the present invention include those disclosed in Brinkman et al. (1995); Ames et al. (1995); Kettleborough et al., (1994); Persic et al., (1997); Burton et al., (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

A “phage display library” means a collection of phage that have been genetically engineered to express a set of putative targeting moieties (e.g., peptides or polypeptides) on their outer surface. In preferred embodiments, DNA sequences encoding the putative targeting moieties are inserted in frame into a gene encoding a phage capsule protein. In other preferred embodiments, the putative targeting moieties are in part random mixtures of all twenty amino acids and in part non-random.

In preferred embodiments, selection of phage that target an organ, a tissue or a cell type is achieved using the BRASIL (Biopanning and Rapid Analysis of Soluble Interactive Ligands) technique. In BRASIL, an organ, tissue or cell type is gently separated into cells or small clumps of cells that are suspended in an first, preferably aqueous phase. The aqueous phase is layered over a second, preferably organic phase of appropriate density and centrifuged. Cells attached to bound phage are pelleted at the bottom of the centrifuge tube, while unbound phage remain in the aqueous phase. This allows a more efficient separation of bound from unbound phage, while maintaining the binding interaction between phage and cell. BRASIL may be performed in an in vitro or in vivo protocol, in which organs, tissues or cell types are exposed to a phage display library.

In certain embodiments, a subtraction protocol may be used with BRASIL or other screening protocols to further reduce background phage binding. The purpose of subtraction is to remove phage from the library that bind to cells other than the cell of interest, or that bind to inactivated cells. In alternative embodiments, the phage library may be screened against a control cell line, tissue or organ sample that is not the targeted cell, tissue or organ. After subtraction the library may be screened against the cell, tissue or organ of interest. In another alternative embodiment, an unstimulated, quiescent cell line, tissue or organ may be screened against the library and binding phage removed. The cell line, tissue or organ is then activated, for example by administration of a hormone, growth factor, cytokine or chemokine and the activated cell line screened against the subtracted phage library. Other methods of subtraction protocols are known and may be used in the practice of the present invention, for example as disclosed in U.S. Pat. Nos. 5,840,841, 5,705,610, 5,670,312 and 5,492,807, incorporated herein by reference.

Targeting agents may include without limitation chemical conjugates, lipids, glycolipids, hormones, sugars, polymers (e.g. PEG, polylysine, PEI and the like), peptides, polypeptides, oligonucleotides, vitamins, antigens, lectins, antibodies and fragments thereof. They are preferably capable of recognizing and binding to cell-specific markers, tissue-specific markers, cellular receptors, viral antigens, antigenic epitopes or tumor-associated markers.

### II. Therapeutic and Diagnostic Methods

As we enter an age of engineering molecular assemblies, the various combinations of nanotechnology, phage display, and biophotonics offers remarkable opportunities to improve the diagnosis and treatment of various diseases, such as cardiovascular and pulmonary diseases. The phage-nanoparticle assemblies typically have optical properties, that may include the capability of converting near infrared (NIR) radiation into heat, providing signal enhancement for fluorescent imaging and surface enhanced Raman scattering (SERS) detection. The combination of NIR optical properties and the targeting capabilities of phage allow phage-nanoparticle assemblies to carry targeted therapeutic and possessing detection functions. Au-phage assemblies, as well as other phage-nanoparticle assemblies, may be used in targeted NIR photo-therapies, NIR surface enhanced Raman scattering (NIR-SERS) and nanoparticle assisted fluorescence enhancement. These compositions and methods may be used for novel imaging and detection schemes to be applied for the detection and treatment of cardiovascular disorders, pulmonary disorders, and the like.

In an exemplary embodiment, the phage-nanoparticle assembly may be applied to in vitro and in vivo targeted detection of cells. The targeted detection may be accompanied, for example, by fluorescence enhancement, SERS, or the like. In vitro detection of target cells may also use phage-nanoparticle assemblies as NIR-SERS and/or SERS signal reporters. Phage-nanoparticle assemblies may be brought into proximity to a fluorophore with and enhance signal generated by a fluorophore, or a fluorescing molecule or complex. In some embodiments, phage-nanoparticle assembly may be taken up into a cell or associated with the external cell surface. A phage-nanoparticle assembly, the phage, the nanoparticles or other agents associated with the assembly, may be further functionalize to allow the coupling of other agents to the phage-nanoparticle, such biotin or a cross-linker moieties, which in turn may be coupled to moieties such as targeting peptides or therapeutics.

In certain aspects of the invention, a cell may be contacted with a phage composition and a nanoparticle composition separately and the phage-nanoparticle assembly forms after administration of components to the cell, tissue, organ, organism or sample.

In still further aspects, a therapeutic molecule can be associated with the phage-nanoparticle assembly. The associated therapeutic molecule may then be delivered or concentrated by the localization of the phage-nanoparticle assembly. In other aspects the therapeutic molecule may be activated when the phage-nanoparticle assembly interacts with electromagnetic radiation, e.g., photolytic coupling of the therapeutic agent to the assembly. Thus, not only could the molecule be preferentially localized but it may also be preferentially activated through association with the phage-nanoparticle assembly. In a preferred embodiment the therapeutic molecule is a therapeutic nucleic acid.

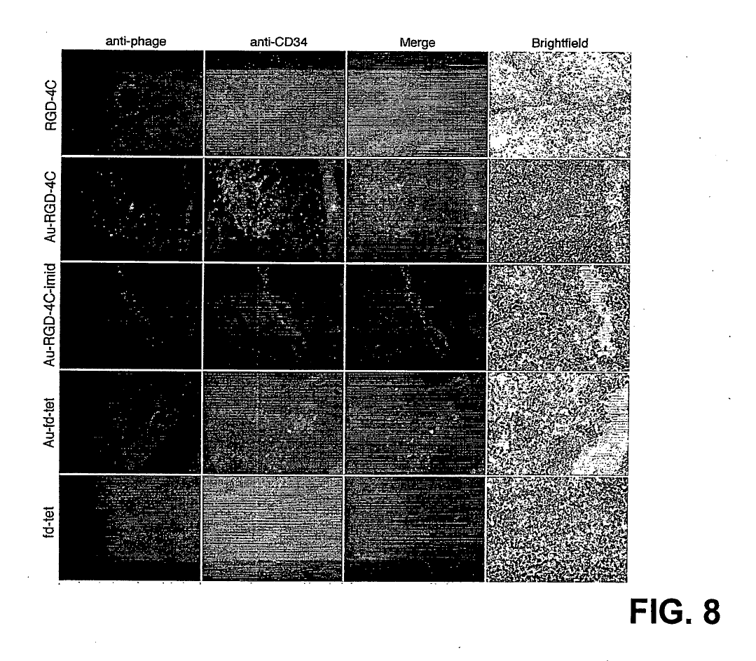

In certain embodiments, the compositions of the invention may be used as Au-phage nanoshuttle for in vivo delivery and detection. Using the melanoma tumor model and the Bk57 mice (FIG. 8), phage based nanoshuttles have been detected. The results with nanoshuttles mirrored the studies using phage only. Nanoshuttles demonstrated better signal with some background associated with the Au-fd-tet nanoshuttle (weak background). The organs showed comparable accumulation or lack off in all systems (brain and pancreas were negative; spleen>liver>lung were positive).

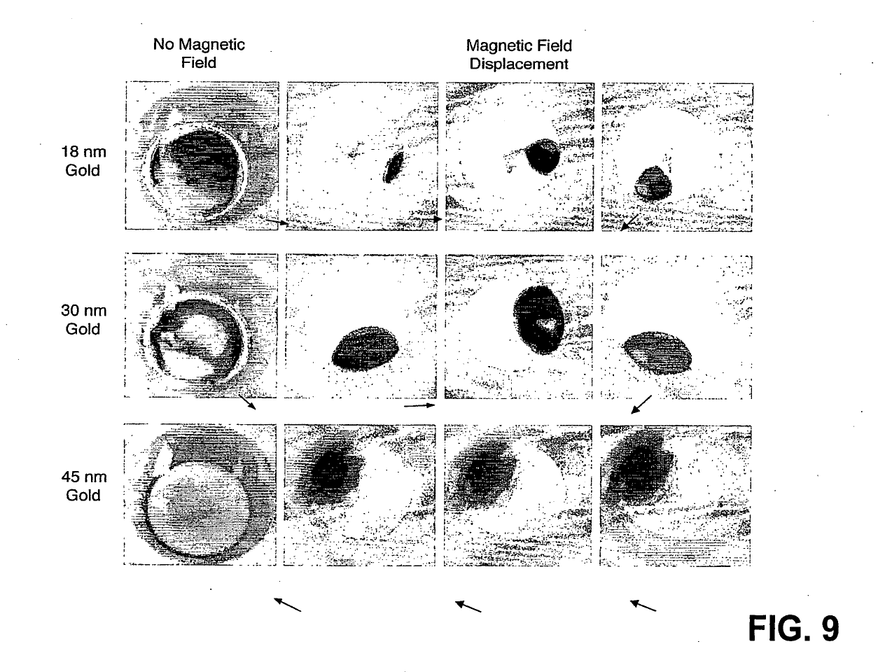

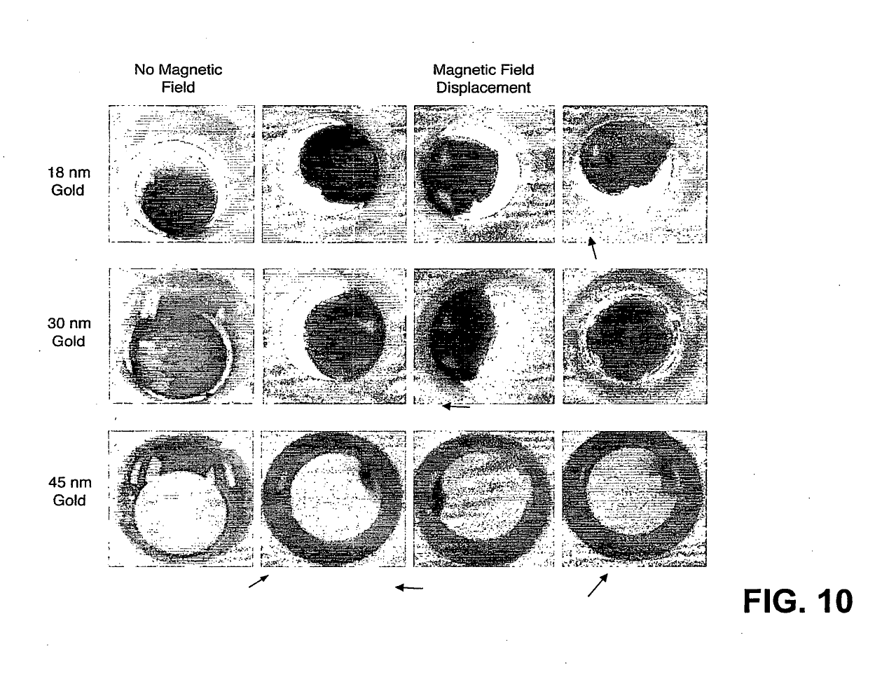

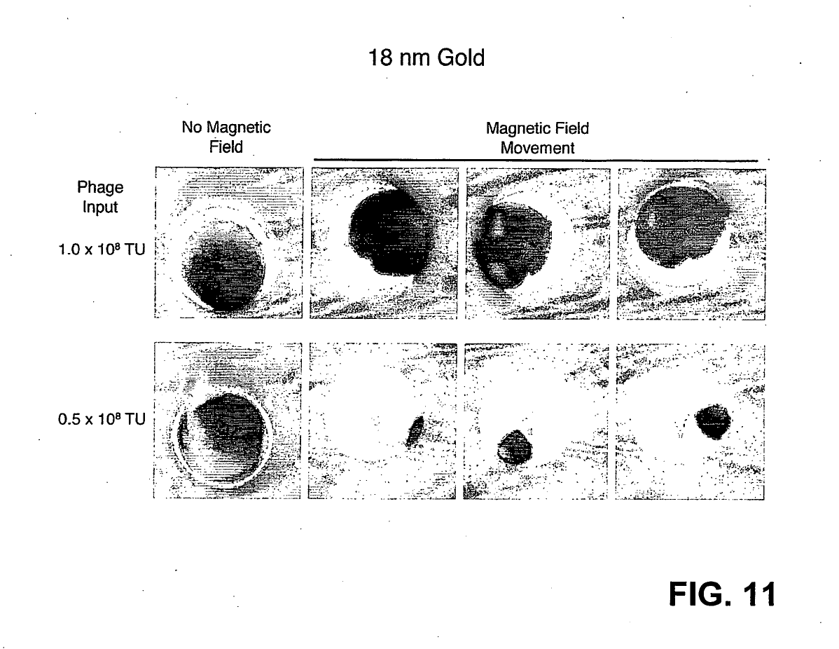

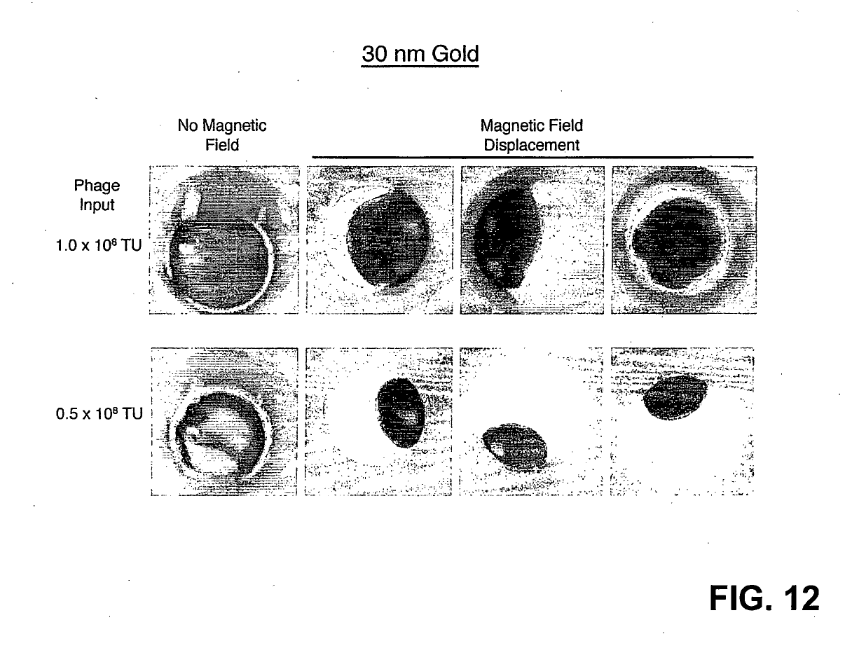

In still further aspects of the invention employ methods and compositions related to iron-phage hydrogel formation (FIGS. 9-12). Optimization studies on hydrogel formation and magnetic response have demonstrated the ability to alter the structure of the hydrogels and how it responds to an applied magnetic field by change nanoparticle size and phage concentration. Exemplary hydrogels were formed in the presence of imidazole.

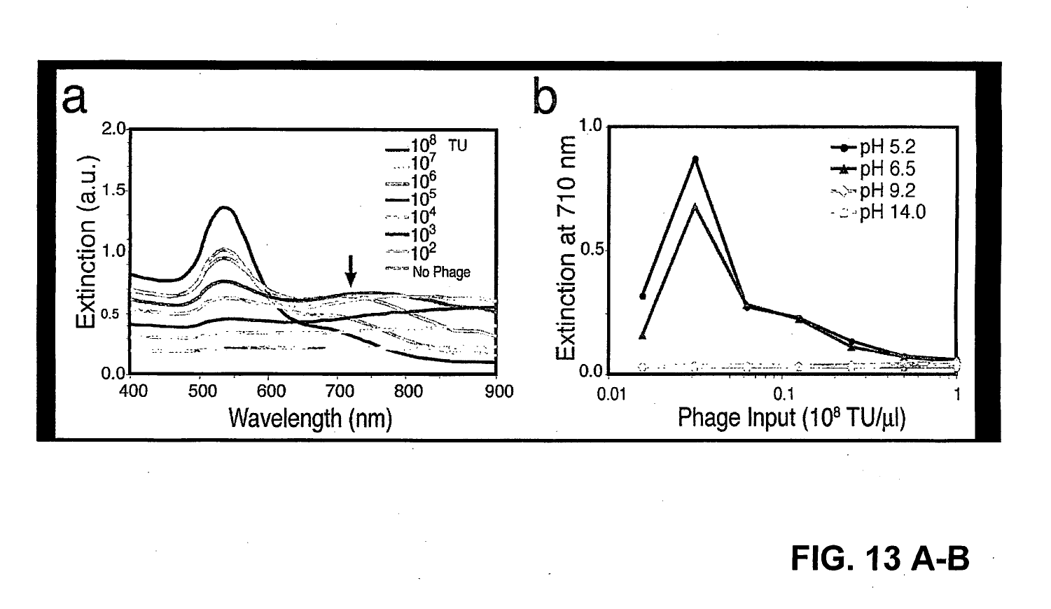

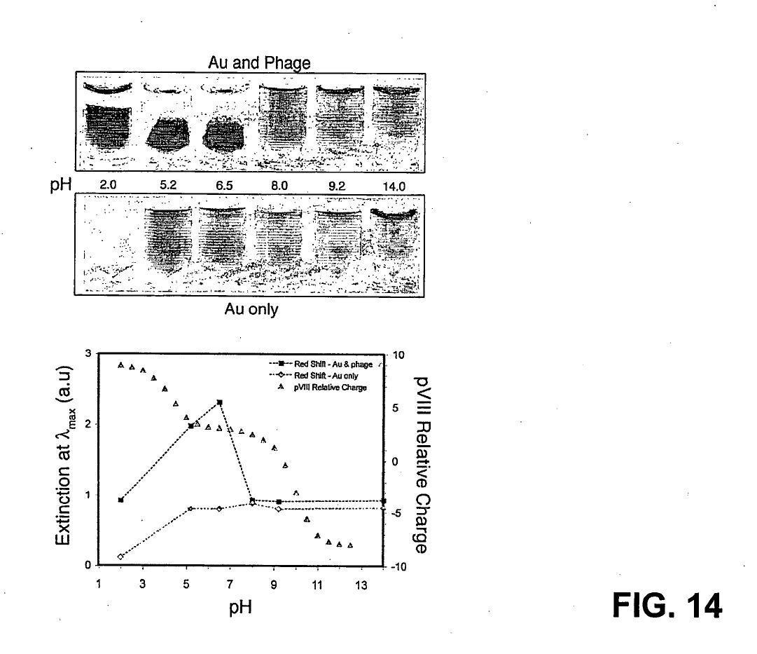

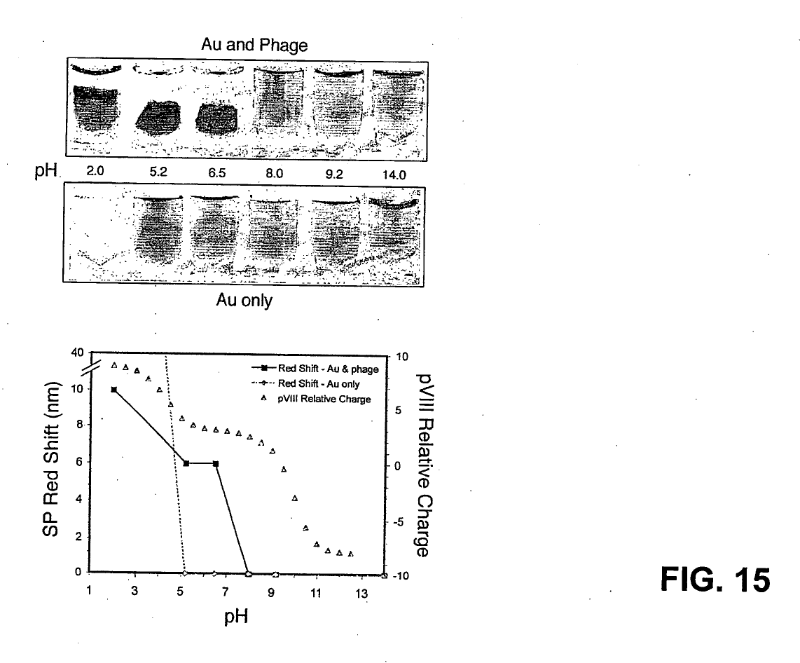

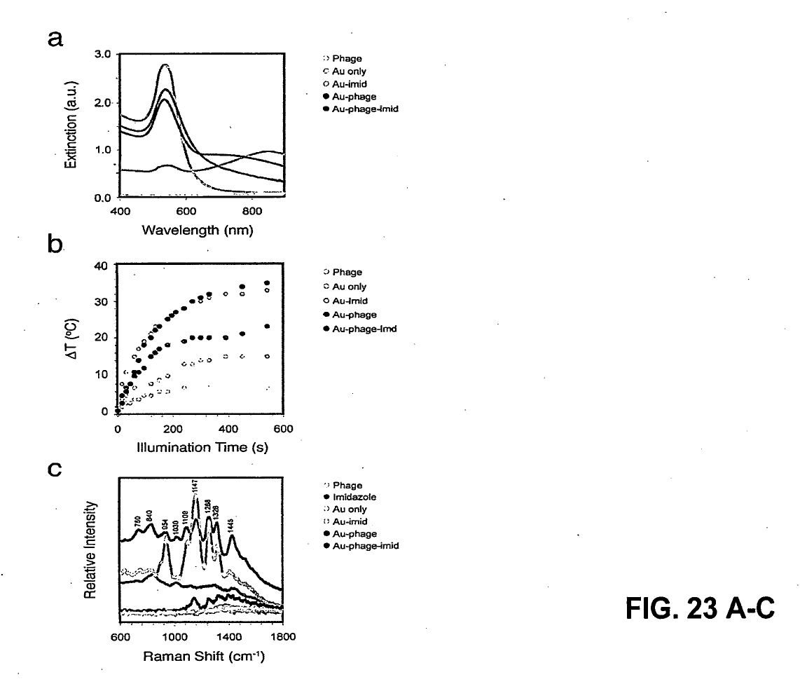

In one example a Au-phage (including iron-phage) assembly mechanism and physical response was assessed (FIGS. 13-16). It is contemplated that the native pVIII major capsid proteins function as the binding sites for the Au-phage network assembly (see FIG. 23C). Given the absence of usual metal binding amino acid residues (such as Cys and His) on the pVIII protein, the self-assembly direct-assembly of Au nanoparticles onto phage should be largely directed by electrostatic interaction. It is well known that gold nanoparticles can be made to agglomerate by varying solution ionic conditions (specifically, agglomeration shows a dependence on ionic strength). In solution, gold nanoparticles are coated with an adsorbed anionic layer; in these exemplary studies these layers are composed of citrate anions resulting from the reductive particle synthesis. Attraction between like-charged particles can occur due to correlated fluctuations in the surrounding ion clouds. Thus, the presence of ions can be used to mediate the agglomeration. For example, gold nanoparticles agglomerate with the addition of salt as indicated by a broadening and shift to longer wavelength in the surface plasmon absorption peak. Phage particles (both fd and M13) also act as polyanionic particles in solution with several negative surface charges associated with each of the ≈2700 copies of the major capsid protein (fd is more anionic than M13 due to the replacement of Asn12 with Asp12). Further, bundles of phage form from like-charge attraction and, analogous to the mediation of gold nanoparticle agglomeration, solubilization of such bundles is dependent on solution ionic strength. Gold agglomeration was found to be induced by 0.25 M NaCl (FIG. 13A), which could be minimized by Au-phage interactions (indicated by the small red shift) when phage input increased. This suggests a similar physical interpretation for binding in the mixed phage/gold systems as that found in the gold-gold and phage-phage binding. This greater immunity to salt when phage was present indicates a greater stability in the gold-phage networks.

Although phage has an overall negative charge at all tested conditions, formation of the Au-phage networks was observed at pHs well below the pI (9.4) of the individual pVIII proteins (calculated with pI calculator from www.scripps.edu/cgi-bin/cdputnam/protcalc). This was determined by comparing network formation as a function of phage input at various pH (FIG. 13B). The Au-phage solutions prepared at lower pH (pH≦6.5) showed a stronger SP red shift (extinction at 710 nm) than solutions prepared at high pH (pH≧9.2). Thus at short distances, the local positive charge of the pVIII proteins along the thin and long phage surface (6 nm×1,000 nm) can interact with the negatively charged spherical Au nanoparticles. In comparison to salt induced aggregation, the sharp increase in extinction (710 nm) resulted from close packing of Au nanoparticles (dAu<2rAu) induced by the titration of phage binding sites in the solution. The drop in extinction signal at the lowest tested phage input is a result of phage depletion, where there are mostly dispersed Au nanoparticles (there was no sign of Au aggregation in the absence of phage at the tested pHs). These short-range interactions, likely, further contribute to the stability of these networks.

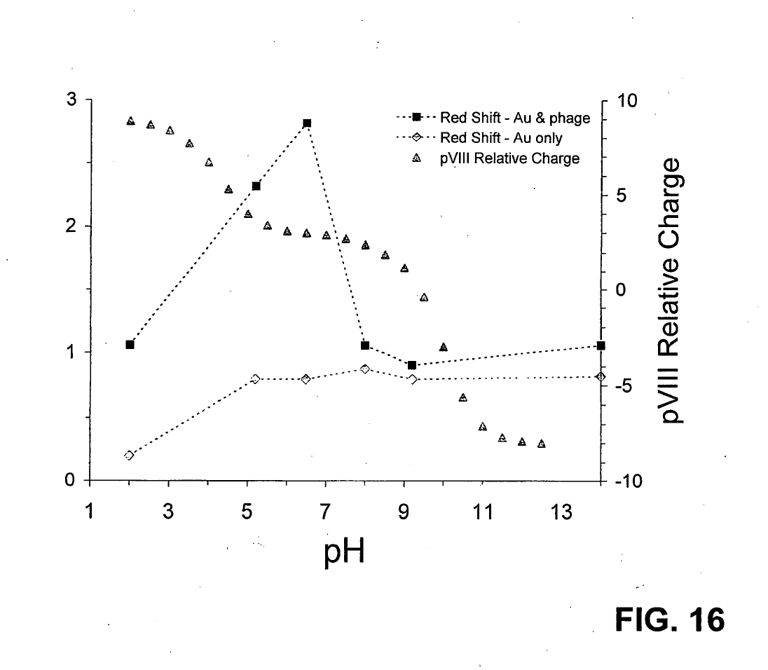

It is not clear what happens to the local organization of the pVIII capsid once the Au interacts with the phage. When dealing with proteins there are a number of physical variables that include, protein interactions, structures unfolding, conformations changes, and translocations to name a few. Although most of the pVIII residues are embedded in the capsid structure, it is not known what happens once the Au is there. It it is contemplated that the negative interaction exist, but when the pVIII is positively charged (at low pH, within a threshold), the pVIII-Au (−/+) attraction/interaction can induce conformational change by locally unfold the capsid, then expose/solvate additional pVIII amino acid residues (better reflecting of the charge vs. pH profile). This likely explain some of the data presented in (FIGS. 16-18). In some aspects of the invention the pH must be driven much lower than the pI to drive hydrogel formation. This could explain the structural changes between the hydrogel at pH 5 and pH 2. Studies on the kinetics of formation, temperature response vs. illumination time, mass/volume ration of hydrogels have been performed.

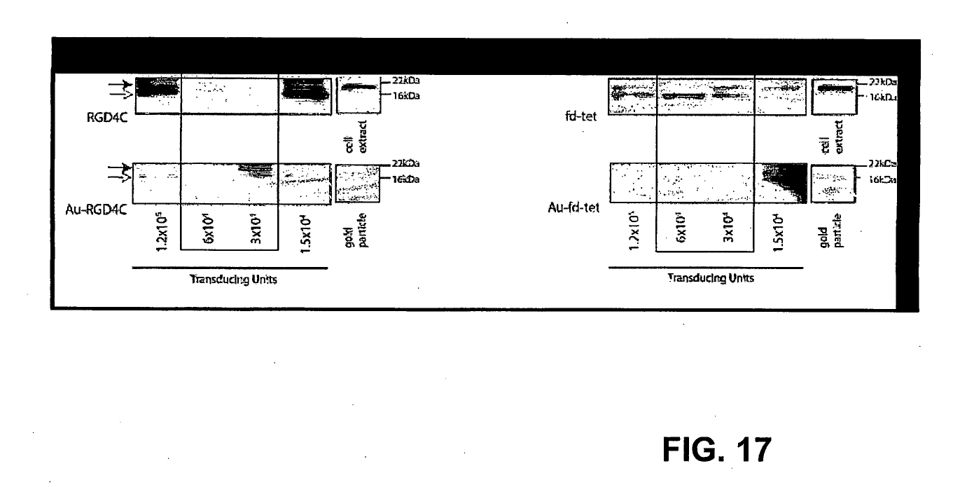

In still a further aspect of the invention stem cells may be used in conjunction with Au-phage hydrogels—Western blot (FIG. 17) Prior studies assessed the expression level between Au—RGD-4C and RGD-4C when cells were incubated overnight. The study described in this paragraph incubate the cells to the point of visual detection of the interaction of the cells with the networks, which is when the hydrogel fibers start to stretch (generally one hour after adding the cells). Interestingly, the cells that show the largest difference in phosphorylation are for the networks that undergo the fastest structural changes (6×10⁴ and 3×10⁴ TU). The rationale was, because the Au—RGD-4C hydrogels are known to start to interact with the cells faster than the Au-fd-tet therefore, if stopped at the point that the Au—RGD-4C hydrogels start to change structure, one would be able to detect phosphorylation differences. FIG. 17 shows a western blot result, where differences in phosphorylation were detected between phage and Au-phage systems. There is a clear low molecular weight phosphorylated band (˜16 kDa, arrow) for the cells cultured in Au—RGD-4C hydrogel, which is not present in the RGD-4C only system (˜16 kDa). The differences in phosphorylation between Au—RGD-4C, RGD-4C and Au-fd-tet were very clear, however the fd-tet system show many phosphorylated bands. The results provide that there are difference in phosphorylation of a low MW Protein (˜16 kDa) between Au—RGD-4C and RGD-4C; there are difference in phosphorylation between Au—RGD-4C and Au-fd-tet; time of incubation is an indicated factor in the phosporylation process; cells (and/or phosphorylation) are sensitive to concentration of phage and/or structure of the hydrogels, there is also visual evidence for this stretching of hydrogel fibers.

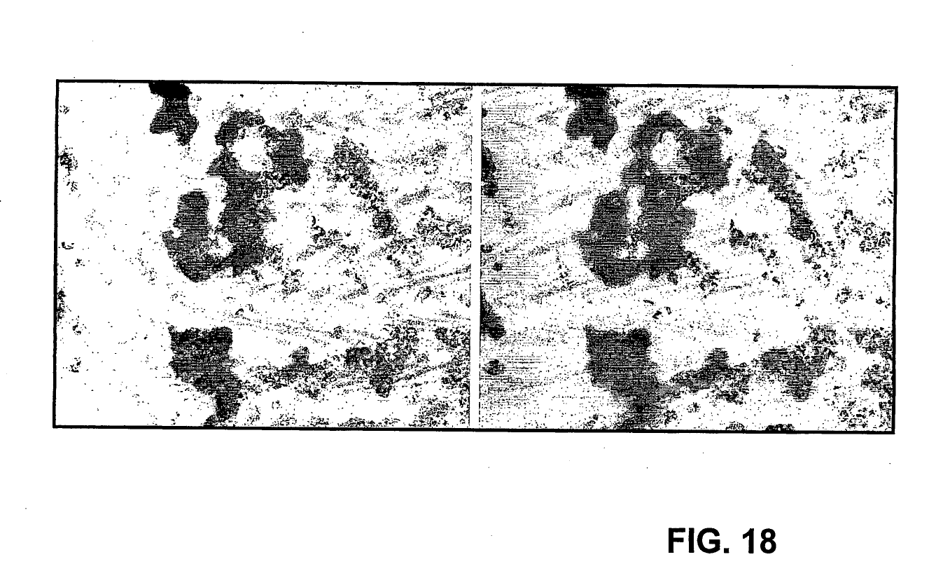

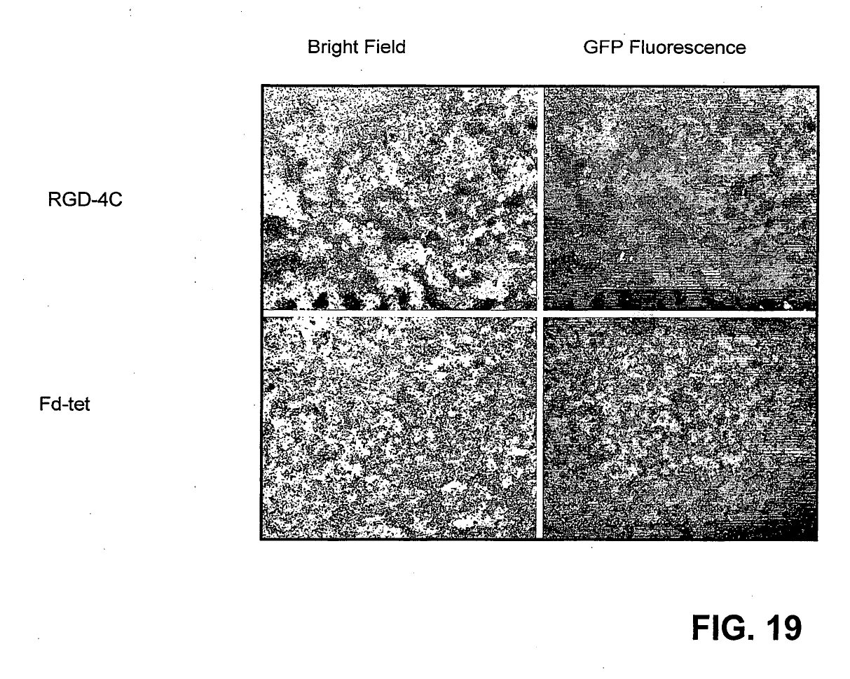

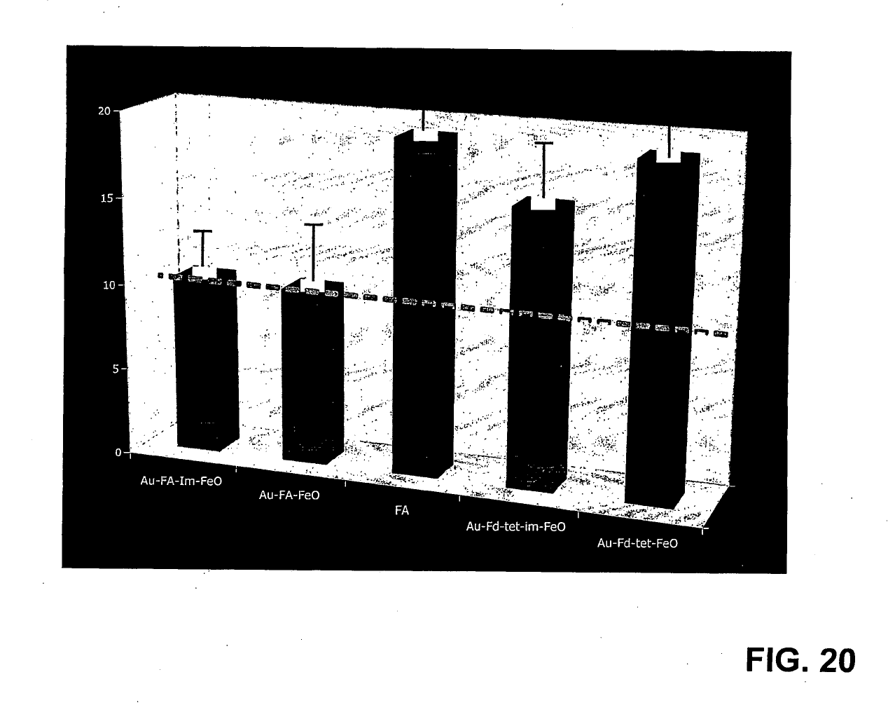

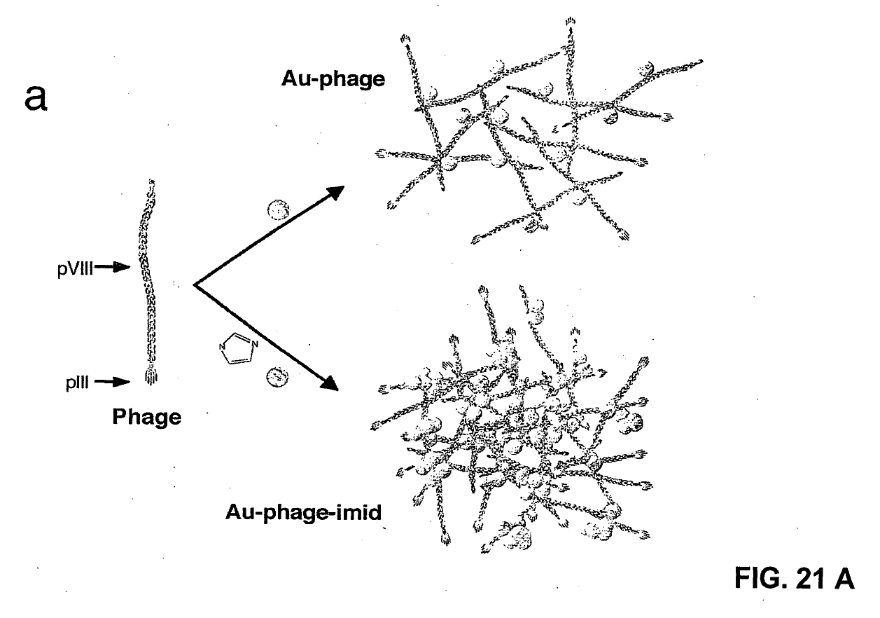

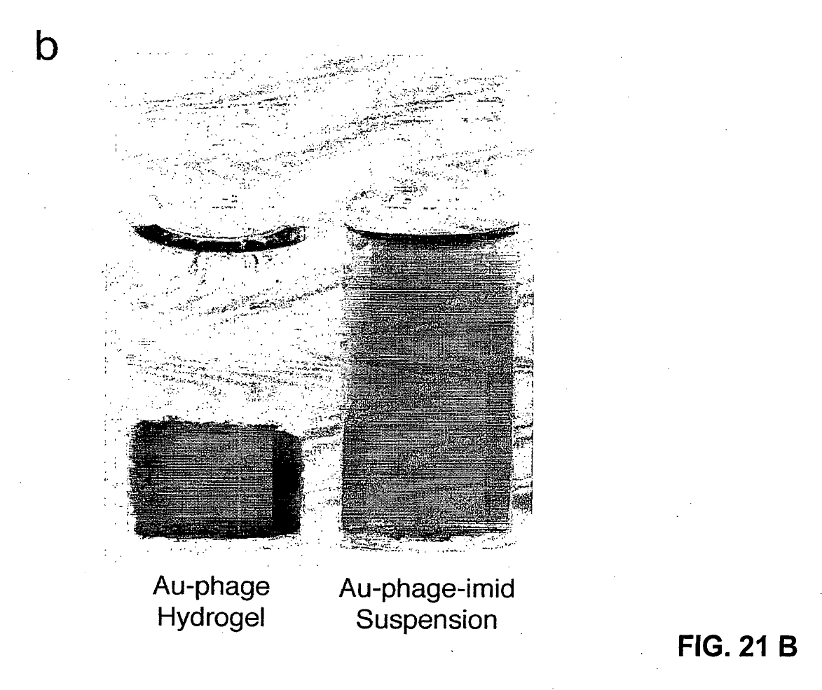

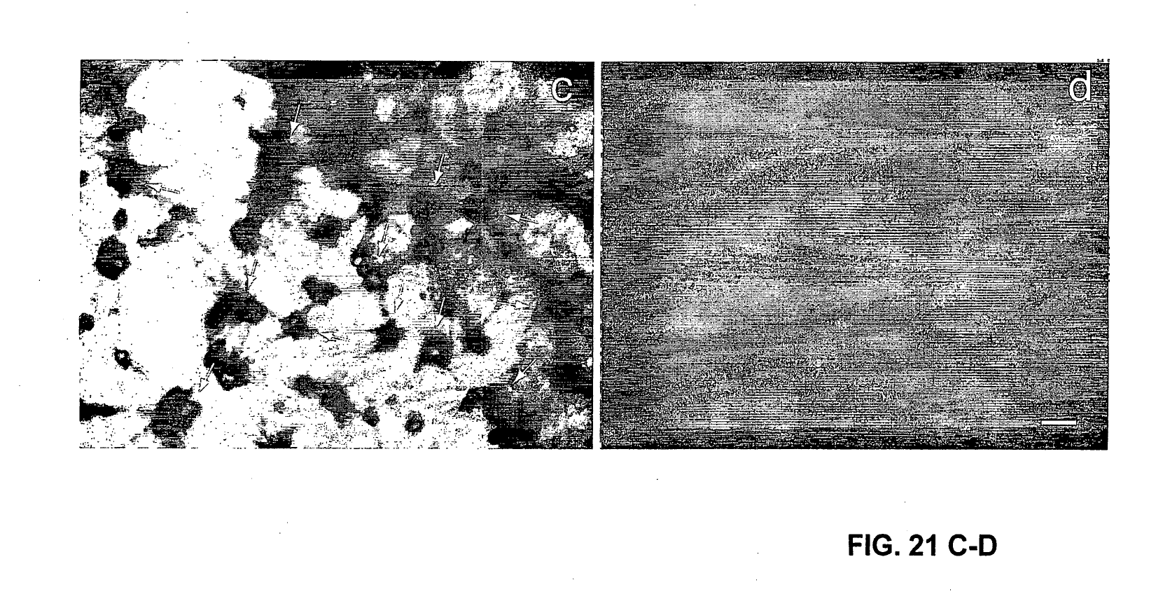

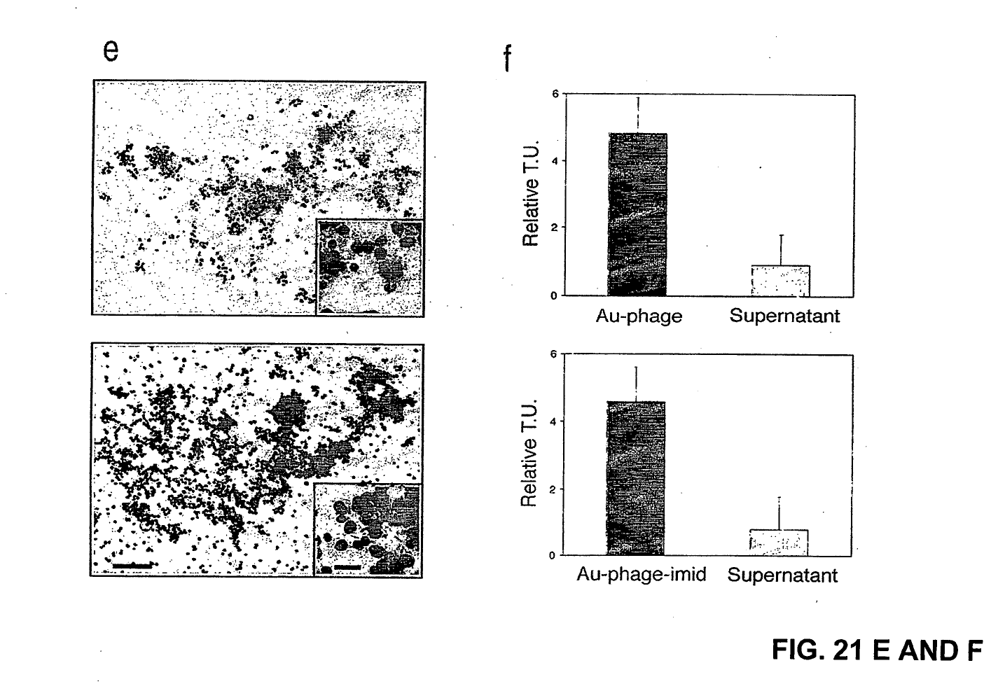

Other aspects of the invention are related to Au-phage hydrogel and gene delivery—Au-phage-GFP (FIGS. 20 and 21). Hydrogels were prepared with “GFP phage” to show that cells are alive within the hydrogel structure. If the cells that are imbedded in the hydrogel structure are expressing GFP, it signifies that the cells are viable and capable of expressing GFP. FIGS. 20 and 21 show the fluorescence from cells (HEC-293) incubated with Au-phage-GFP. The onset of GFP fluorescence occurred after 3 days of incubating the cells within the hydrogels (FIG. 18). After 10 days (FIG. 20; without replenishing the cell growth media) a considerably increase in the number of fluorescing cells was detected relative to day four. At ten days incubation point a significantly larger amount of fluorescent cells were detected for the Au—RGD-4C-GFP hydrogels relative to Au-fd-tet-GFP. In the least, it can be concluded that cells are alive when within the network structures, cells can grow and function (express GFP) within in the presence and within the network structure, and hydrogels are degraded by the cells (biodegradable).

Additional aspects include MRI T2 measurements of iron-phage hydrogel formation (FIG. 20). FIG. 20 is the plot of the T2 for the fat tissue from Balb-C mice injected with the different phage and Au-phage-FeO (4 hr circulation, then fat and organs removed). T2 is the nuclear spin relaxation constant that is used to give the best image contrast when using iron oxide nanoparticles. The presence of FeO decreases the T2 time for nuclei surrounding FeO. The largest T2 change (decrease) was for the tissue injected with Au—FA-imd-FeO, as expected, since this Au-phage had the lowest T2 of all the other particles. The result of the fat analysis behaved as expected as far as T2 magnitude: T2 for Au—FA-imd-FeO<Au—FA-FeO<Au-fd-tet-imid-FeO<Au-fd-tet-FeO<FA. Here, the T2 was determined by measuring the T2 from different regions of fat tissue in a conical tube. A larger volume does not effect directly the measurements, it just makes less certain and laboring during the analysis.

T2 definition: The relaxation process in nuclear magnetic resonance is controlled by the parameters T1 and T2. These parameters can be tissue dependent, introducing the capability to differentiate tissue types. T2 is the spin-spin relaxation time, which is the time constant for the relaxation process in the projected xy-plane. In contrast, T1 is the time constant in the z-direction. T1 and T2 are related to physical interaction phenomena's from atomic nuclei and its surroundings. The T2 effect in the relaxation process is due to dephasing of the individual protons because of the existence of a varying magnetic field. Each proton will experience the external, stationary magnetic field B0 along with the self generated magnetic field of the neighboring protons. Since the angular frequency of a proton is proportional to the applied magnetic field, the protons will respond at different frequencies, depending on the field strength and direction. When the protons respond at different frequencies, some nuclei will be out of phase relative to the incident wave. This off phase phenomenon, the Larmor frequency, results in a net decrease in the magnetic moment in the xy-plane as a function of time (changes in T2). The time from maximum magnetic moment to zero is characterized by T2. T2 depends on the mobility of the protons, a large mobility results in an average magnetic field variation of zero, resulting in a long T2. (es.oersted.dtu.dk/˜masc/T1_T2.htm).

A. Detecting Phage-Nanoparticle Assemblies

The Inventors have generated exemplary stable, biologically active networks of self-assembled phage-nanoparticle assemblies, in which one is able to control or tune the chemical and physical properties of these biological structures. This tuning capability combined with the tissue targeting properties (selected or engineered) of phage may allow the integration of multiple functions in a single nano-assembly, serving as a complementary and non-mutually exclusive tool among applications, i.e., near infrared (NIR) surface enhanced Raman scattering (SERS) detection, enhanced fluorescence imaging, and/or heat deposition for NIR photo-therapy. In various embodiments of the invention, phage-nanoparticle assemblies may be detected by Raman spectroscopy, for example by surface enhanced Raman spectroscopy (SERS), surface enhanced resonance Raman spectroscopy (SERRS), coherent anti-Stokes Raman spectroscopy (CARS) or other known Raman detection techniques.

The compositions and methods described herein may be used, for example, in cell specific chemical and physical identification by detection of elastic and/or inelastic scattered light. The methods may use an unique optical configuration for in solution detection for identification of cells based on cell specific SERS signal. A combination of nanoparticles (e.g., Au nanoparticles) and phage may be used for labeling and targeting of assemblies that provide for cell identification using Raman scattering and the like.

1. Raman Scattering

Briefly, when light encounters molecules in the air, the predominant mode of scattering is elastic scattering, called Rayleigh scattering. It is also possible for the incident photons to interact with the molecules in such a way that energy is either gained or lost so that the scattered photons are shifted in frequency. Such inelastic scattering is called Raman scattering.

Raman scattering depends upon the polarizability of the molecules. For polarizable molecules, the incident photon energy can excite vibrational modes of the molecules, yielding scattered photons which are diminished in energy by the amount of the vibrational transition energies. A spectral analysis of the scattered light under these circumstances will reveal spectral satellite lines below the Rayleigh scattering peak at the incident frequency. Such lines are called “Stokes lines.” If there is significant excitation of vibrational excited states of the scattering molecules, then it is also possible to observe scattering at frequencies above the incident frequency as the vibrational energy is added to the incident photon energy. These lines, generally weaker, are called anti-Stokes lines.

2. Near Infrared (NIR) Detection

NIR detection, imaging and therapy are expected to have a major impact on biotechnology and medicine because biological tissues show minimal NIR radiation (700-900 nm) absorption (Lin et al., 2002), allowing efficient light penetration for in vivo imaging and phototherapy applications. It has been shown that the proximity of nanoparticles within a cluster induces a shift in the maximum absorption wavelength of the nanoparticle plasmon from the visible into the NIR wavelengths. This NIR absorption allows the energy conversion of the absorbed NIR photons into heat. Such properties can be utilized for targeted tissue ablation at the cellular level by localized heating. NIR imaging and phototherapy have been attempted by using NIR fluorescence reporters, such as indocyanine green (ICG), but such molecules show poor biocompatibility and sensitivity (Lin et al., 2002; Desmettre et al., 2000; Mordon et al., 1998; Mordon et al., 1997a; Mordon et al., 1997b; Mordon et al., 1996). In order to be effective, NIR reporters have to be sensitive, specific, and compatible for in vivo use.

Further motivation for the use of NIR and SERS spectroscopy/microscopy is its Raman scattering high chemical selectivity and sensitivity when combined with nanoparticles and NIR radiation (Cao et al., 2002; Crow et al., 2003; Grubisha et al., 2003; Hartschuh et al., 2003; Kneipp et al., 1999; Nie and Emory, 1997). NIR-SERS labels may have superior optical properties and biocompatibility over NIR fluorescent labels. SERS molecular labels based on the assembly of nanoparticles (e.g., Au nanoparticles) and imidazole provide, in some instances, approximately 10⁸ Raman signal surface enhancements (relative to “nanoparticle free” imidazole). The nanoparticle-imidazole labels may be assembled onto the surface of phage particles that provide the tissue targeting capability required for the development of guided delivery of imaging and therapeutic agents. The strong affinity linking imidazole and nanoparticles induces the assembly of the nanoparticles, where the proximity of nanoparticles within a cluster induces a shift in surface plasmon absorption into the NIR wavelengths. The NIR absorption shift provides the energy conversion of the absorbed NIR photons into heat (Hirsch et al., 2003), and the Raman/fluorescence signal surface enhancement necessary for non-invasive in-tissue detection.

The nanoparticle-imidazole assemblies optical and light-to-heat conversion properties are analogous to the properties of Au—Si nanoshells (nanoshells) (Averitt et al., 1997). However, research in the application of nanoshells have shown slow progress in integrating tissue-targeting capability. Unfortunately, the nanoshell in vivo targeting mechanism relies on their diffusion through “leaky” vasculature (O'Neal et al., 2004). Therefore, a substantial limitation of that system is that there is no receptor-mediated cell targeting, and the accumulation of nanoshells in the liver and kidney leads to serious complications, such as kidney failure (Gatti and Rivasi, 2002; Sharma and McQueen, 1980).

B. Surface Enhanced Raman Scattering (SERS) Methods

SERS can be used for detecting cell binding by phage-nanoparticle assemblies. The SERS spectra of cells (e.g., cells suspended in an aqueous medium) incubated with phage-nanoparticle assemblies may be measured. The SERS signal can be evaluated using a fiber-optic probe to deliver laser light (e.g., 785 nm) and to collect the Raman signal. Nanoparticle assemblies are often used to generate the surface enhancement required for SERS detection. SERS enhancement is typically on the order of 10⁶-10⁹ stronger than normal Raman scattering, making SERS sensitivity comparable to fluorescence. The SERS spectra can discriminate events related to phage binding to cells. Signal intensity typically correlates to levels of cell binding and internalization.

Near infrared (NIR) SERS may be used in vivo for non-invasive detection of injected phage-nanoparticle assemblies by detecting enhanced Raman signal. A fiber-optic probe with a defined focal length, such as 7 mm, may be positioned at an appropriate distance (e.g., 3 mm) from the target area (e.g., cell mass to be diagnosed) to deliver and collect light through the skin. NIR radiation and SERS detection using phage-nanoparticle assemblies as SERS signal reporters can be developed for in vivo imaging. A portable R2001 Raman spectrometer is one example of detection device that can be used in this and other contexts of the invention.

C. Cell Internalization and Fluorescence Enhancement

In other aspects of the invention, the compositions of the invention may be used in live cells. Binding assays can be performed in which the phage-nanoparticle assemblies are visualized by immunofluorescence. Cells may internalize the phage-nanoparticle assemblies, as exemplified by the internalization of RGD-4C phage which target αᵥ-integrin cell surface receptors. In particular aspects, internalized assemblies may be detected. The fluorescence enhancement likely results from the combination of surface enhancement provided by the phage-nanoparticle assembly and the receptor-mediated internalization.

In another aspect of the invention, detection tools for study and diagnosis of various biological systems, such as lung vasculature are contemplated. In vitro and in vivo studies may be performed by using the phage-nanoparticle assembly with or without an organizing agent to target a particular cell type, such as lung epithelial cells (LECs). Typically the targeting phage-nanoparticle assemblies are visualized by confocal microscopy. One can control phage localization by manipulating the nanostructure of the assembly, as well as incorporating or selecting targeting functions of the assembly components. Compositions of the invention may also be use for in vivo targeting of cells, tissues, vasculature, organs, or organisms.

D. Cell Sorting

In various embodiments, the phage-nanoparticle assembly may be used in methods of Raman activated cell-sorting (RACS). RACS combines Raman scattering, phage, and nanoparticles to detect and sort cells. Such a device can be used with various cell-sorting techniques, such as flow cytometry and microscopy. RACS may be divided in three parts: a) instrumentation detection scheme allowing elastic (angle dependent light scattering, ADLS) and inelastic (Raman) scattering detection in liquid and flowing systems, b) surface enhancement Raman scattering (SERS) for direct detection of cells using nanoparticles; c) and a the use of phage as targeting and labeling entity for Raman detection. Part (a) typically incorporates a unique optical configuration which utilizes an ellipsoidal mirror and an fiber-optical probe to integrate simultaneous detection of elastic and inelastic scattering, respectively. This optical configuration is ideal for flow cytometry instrumentation, where ADLS portion identifies cells based on their physical properties, such as shape, optical properties and size, and the Raman differentiates cells based on its chemical composition. Parts (b) and (c) enhances the cell sorting capabilities of (a), taking advantage of nanoparticles and phage as unique labeling and targeting agents for a wide range of Raman techniques, such as SERS, coherent anti-stoke Raman scattering (CARS) and resonance Raman detection.

The combination of elastic and inelastic scattering provide great improvement in characterizing cells when compared to fluorescence assisted cell sorting (FACS). The inelastic and Raman scattering signal from cells allow simultaneous detection of the physical and chemical characteristics of a cell. Because Raman scattering cross section of most molecules is generally small, nanoparticles and phage are introduced to provide enhancement of Raman signals by SERS, CARS, resonance Raman or combinations thereof. The chemical specificity of all Raman detection techniques and molecular diversity of phage provide a larger number of possible targeting and labeling for Raman detection. Further, the application of SERS using phage-nanoparticle assemblies allow one to utilize the NIR range of the optical spectrum, where NIR optoelectronics (high-power laser diodes, efficient fiber-optics and sensitive CCD detectors) provide the capabilities for building compact and relatively inexpensive instrumentation. FACS is not effective in the NIR light spectrum region because NIR flurophores show poor performance.

E. Isolated Nucleic Acids for Therapy

Phage-nanoparticle assemblies of the invention may be used to deliver therapeutic nucleic acids and/or therapeutic compositions. Certain genes may be used therapeutically by increasing or decreasing the expression of the gene or activity of an encoded protein in a cell. Other genes related to resistance of a cell to a therapy may be down regulated transcriptionally or inhibited at the protein level by various therapies or products of a therapeutic nucleic acid (both proteinaceous and nucleic acid products), such as anti-sense or interfering nucleic acid methods. Therapeutics that target the transcription of a gene, translation of RNA, and/or activity of an encoded protein may be used as a therapy, or in other aspects, may be used as a primary therapeutic apart from or in combinations with other therapies.

Nucleic acids of the present invention include nucleic acid isolated from a sample, probes, or expression vectors for both analysis and therapy. Certain embodiments of the present invention include the evaluation of the expression of one or more nucleic acids. In certain embodiments, wild-type, variants, or both wild-type and variants of these sequences are employed. In particular aspects, a nucleic acid encodes for or comprises a transcribed nucleic acid. In certain aspects, an expression cassette may be incorporated into a phage particle of the assembly or in another delivery vector operatively coupled to an assembly of the invention.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). “Nucleic acid” encompass the terms “oligonucleotide” and “polynucleotide,” each as a subgenus of the term “nucleic acid.” The term “oligonucleotide” refers to a molecule of between about 8 and about 100 nucleobases in length. The term “polynucleotide” refers to at least one molecule of greater than about 100 nucleobases in length.

In, certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. The term “gene” includes both genomic sequences, RNA or cDNA sequences or smaller engineered nucleic acid segments, including non-transcribed nucleic acid segments, including but not limited to the non-transcribed promoter or enhancer regions of a gene. Smaller engineered nucleic acid segments may encode proteins, polypeptides, peptides, fusion proteins, mutants and the like.

A polynucleotide of the invention may form an “expression cassette.” An “expression cassette” is polynucleotide that provides for the expression of a particular transcription unit. A transcription unit may include promoter elements and various other elements that function in the transcription of a gene or transcription unit, such as a polynucleotide encoding all or part of a therapeutic protein. An expression cassette may also be part of a larger replicating polynucleotide or expression vector. In particular aspects an expression cassette may be packaged in or associated with a bacteriophage, which in turn may be associated with a phage-nanoparticle assembly.

“Isolated” or “isolated substantially away from other coding sequences” means that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

1. Expression Constructs

Expression constructs of the invention may include nucleic acids encoding a protein or polynucleotide for use in treating a disease, e.g., cancer. In certain embodiments, genetic material may be manipulated to produce expression cassettes and expression constructs that encode the nucleic acids or inhibitors of the nucleic acids of the invention. Throughout this application, the term “expression construct” is meant to include any type of genetic construct containing a nucleic acid coding for gene products in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of therapeutic genes or nucleic acids.

A therapeutic vector of the invention comprises a therapeutic gene for the prophylatic or therapeutic treatment of a disease condition. In order to mediate the expression of a therapeutic gene in a cell, it will be necessary to transfer the therapeutic expression constructs into a cell, for example, using phage-nanoparticles of the invention.

Various methods and compositions for nucleic acid transfer may be used in conjunction with the phage-nanoparticle assemblies of the invention, examples of both ex vivo and in vivo may be found in the following references: Carter and Flotte, 1996; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996; Xiao et al., 1996; McCown et al., 1996; Ping et al., 1996; Ridgeway, 1988; Baichwal and Sugden, 1986; Coupar et al., 1988. Other methods of gene transfer include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), naked DNA expression construct (Klein et al., 1987; Yang et al., 1990), Liposomes (Ghosh and Bachhawat, 1991; Radler et al., 1997; Nicolau et al. 1987; Kaneda et al., 1989; Kato et al., 1991) and receptor-mediated transfection (Wu and Wu, 1987; Wu and Wu, 1988).

2. Control Regions

Expression cassettes or constructs of the invention, encoding a therapeutic gene will typically include various control regions. These control regions typically modulate the expression of the gene of interest. Control regions include promoters, enhancers, polyadenylation signals, and translation terminators. A “promoter” refers to a DNA sequence recognized by the machinery of the cell, or introduced machinery, required to initiate the specific transcription of a gene. In particular aspects, transcription may be constitutive, inducible, and/or repressible. The phrase “under transcriptional control” means that the promoter is in the correct location and orientation in relation to the nucleic acid to control RNA polymerase initiation and expression of the gene.

In various embodiments, the human cytomegalovirus immediate early gene promoter (CMVIE), the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral, retroviral or mammalian cellular or bacterial phage promoters, which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic. For example, the ecdysone system (Invitrogen, Carlsbad, Calf.) and Tet-Off™ or Tet-On™ system (Clontech, Palo Alto, Calif.) are two such systems.

In some circumstances, it may be desirable to regulate expression of a transgene in a therapeutic expression vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, the following promoters may be used to target gene expression in other tissues.

Tumor specific promoters such as osteocalcin, hypoxia-responsive element (HRE), MAGE-4, CEA, alpha-fetoprotein, GRP78/BiP and tyrosinase may also be used to regulate gene expression in tumor cells.

It is envisioned that any of the above promoters alone or in combination with another may be useful according to the present invention depending on the action desired. In addition, this list of promoters should not be construed to be exhaustive or limiting, those of skill in the art will know of other promoters that may be used in conjunction with the promoters and methods disclosed herein.

Enhancers may also be utilized in construction of an expression vector. Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Polyadenylation signals may be used in therapeutic expression vectors. Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

3. Therapeutic Genes

The present invention contemplates the use of a variety of different therapeutic genes. For example, genes encoding enzymes, hormones, cytokines, oncogenes, receptors, ion channels, tumor suppressors, transcription factors, drug selectable markers, toxins, various antigens, anti-sense polynucleotide and other inhibitors of gene expression are contemplated for use according to the present invention. In certain embodiments, a therapeutic gene may encode an anti-sense polynucleotide, siRNA, or ribozymes that interfere with the function of DNA and/or RNA. The presence or expression of such a polynucleotide or derivative thereof in a cell will typically alter the expression or function of cellular genes or RNA.

4. Multigene Constructs and IRES

In certain embodiments of the invention, the use of internal ribosome binding sites (IRES) elements are used to create multigene, polycistronic messages. IRES elements are able to bypass the ribosome scanning model of 5′-methylated, Cap-dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple genes can be efficiently expressed using a single promoter/enhancer to transcribe a single message. Any heterologous open reading frame can be linked to IRES elements. This includes genes for therapeutic proteins and selectable markers. In this way, expression of several proteins can be simultaneously engineered into a cell with a single construct and a single selectable marker.

5. Preparation of Nucleic Acids

An isolated nucleic acid may be made by any technique known to one of ordinary skill in the art, such as for example, chemical synthesis, enzymatic production, or biological production. Non-limiting examples of a synthetic nucleic acid (e.g., a synthetic oligonucleotide), include a nucleic acid made by in vitro chemical synthesis using phosphotriester, phosphite, or phosphoramidite chemistry; and solid phase techniques such as described in EP 266 032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotides may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which are incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant. DNA vector replicated in bacteria (see for example, Sambrook et al. 2001, incorporated herein by reference).

A nucleic acid may be purified on polyacrylamide gels, cesium chloride centrifugation gradients, affinity columns, or by any other means known to one of ordinary skill in the art (see for example, Sambrook et al., 2001, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

### III. Devices of the Invention

Exemplary devices disclosed herein may comprise a detection unit that is designed to detect and/or quantify phage-nanoparticle assemblies or derivatives thereof by Raman spectroscopy. Raman detection of nucleotides at the single molecule level has been described previously in U.S. Provisional Patent application 20040110208, which is incorporated herein by reference. Variations on surface enhanced Raman spectroscopy (SERS), surface enhanced resonance Raman spectroscopy (SERRS) and coherent anti-Stokes Raman spectroscopy (CARS) have also been described. In SERS and SERRS, the sensitivity of the Raman detection is enhanced by a factor of 10⁶ or more for molecules adsorbed on roughened metal surfaces, such as silver, gold, platinum, copper or aluminum surfaces. A non-limiting example of a Raman detection unit is disclosed in U.S. Pat. No. 6,002,471, which is incorporated herein by reference.

Excitation of the phage-nanoparticle assemblies may be accomplished by using a variety of sources. For example, an excitation beam may be generated by an Nd:YAG laser at 532 nm wavelength, a Ti:sapphire laser at 365 nm wavelength or other laser source. Pulsed laser beams or continuous laser beams may be used. An excitation beam may pass through confocal optics and a microscope objective, and may be focused onto a microchannel or specimen containing phage-nanoparticle assemblies. The Raman emission light from the cell or other target may be collected by the microscope objective and confocal optics and coupled to a monochromator for spectral dissociation. The confocal optics may include a combination of dichroic filters, barrier filters, confocal pinholes, lenses, and mirrors for reducing the background signal. Standard full field optics may be used as well as confocal optics. The Raman emission signal may be detected by a Raman detector, which may include an avalanche photodiode interfaced with a computer for counting and digitization of the signal.

Alternative examples of detection units are disclosed, for example, in U.S. Pat. No. 5,286,403, including a Spex Model 1403 double-grating spectrophotometer equipped with a gallium-arsenide photomultiplier tube (RCA Model C31034 or Burle Industries Model C3103402) operated in the single-photon counting mode. The excitation source may comprise a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 mu (U.S. Pat. No. 6,174,677). The excitation beam may be spectrally purified with a bandpass filter (Corion) and may be focused on a microchannel or specimen using a objective lens. The objective lens may be used to both excite the phage-nanoparticle and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) may be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors may be used, such as charged injection devices, photodiode arrays or phototransistor arrays.

Any suitable form or configuration of Raman spectroscopy or related techniques known in the art may be used for detection of phage-nanoparticle assembly, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

A device of the invention may include various information processing and control systems and data analysis. The type of information processing system used is not limiting. An exemplary information processing system may incorporate a computer comprising a bus for communicating information and a processor for processing information. The processor may be selected from the Pentium. family of processors made by Intel Corp. (Santa Clara, Calif.) or various other types of processors.

The detection unit may be operably coupled to the information processing system. Data from the detection unit may be processed by the processor and data stored in the main memory. The processor may analyze the data from a detection unit and/or compare the emission spectra from phage-nanoparticle assembly in a microchannel, from a specimen, or from a subject to identify the phage-nanoparticle assembly, its location, distribution, and/or concentration.

While the processes described herein may be performed under the control of a programmed processor, the processes may also be fully or partially implemented by any programmable or hardcoded logic, such as Field Programmable Gate Arrays (FPGAs), TTL logic, or Application Specific Integrated Circuits (ASICs), for example. Additionally, the disclosed methods may be performed by any combination of programmed general purpose computer components and/or custom hardware components.

Following the data gathering operation, the data may be reported to a data analysis operation. To facilitate the analysis operation, the data obtained by the detection unit may be analyzed using a digital computer. The computer may be programmed for receipt and storage of the data from the detection unit as well as for analysis and reporting of the data gathered. Custom designed software packages may be used to analyze the data obtained from the detection unit.

### IV. Pharmaceutical Compositions

Where clinical applications are contemplated, it may be necessary to prepare pharmaceutical compositions—phage-nanoparticle assemblies, phage stocks, proteins, antibodies, organizing agents and/or therapeutic agents—in a form appropriate for the intended application. Generally, this will entail preparing compositions that are essentially free of impurities that could be harmful to humans or animals.

One generally will desire to employ appropriate salts and buffers to render compositions stable and allow for administration of the inventive compositions and/or uptake or association by target cells in a tissue, organ, or organism, e.g., a human subject or patient. Aqueous compositions of the present invention may comprise an effective amount of a phage-nanoparticle assembly, a protein, a peptide, an antibody, a fusion protein and/or other herapeutic agents dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the phage-nanoparticle assembly, proteins or peptides of the present invention, its use in therapeutic compositions is contemplated. Supplementary active ingredients also can be incorporated into the compositions.

The active compositions of the present invention may include classic pharmaceutical preparations. Administration of these compositions according to the present invention are via any common route so long as the target tissue is available via that route. This includes injection, perfusion, aerosol, oral, nasal, buccal, or topical. Alternatively, administration may be by orthotopic, intradermal, subcutaneous, intramuscular, intraperitoneal, intraarterial or intravenous injection. Such compositions normally would be administered as pharmaceutically acceptable compositions, as described herein.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it is preferable to include isotonic agents, for example, sugars or sodium chloride. Prolonged absorption of the injectable compositions can be brought about by the use in the compositions of agents delaying absorption, for example, aluminum monostearate and gelatin.

Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

### V. Kits

Any of the compositions described herein may be comprised in a kit. In a non-limiting example, a phage, a nanoparticle and/or additional agents, may be comprised in a kit. The kits will thus comprise, in suitable container means, phage, nanoparticles (e.g., gold particle) and/or various other additional agents.

The kits may comprise a suitably aliquoted phage, nanoparticle, phage-nanoparticle assembly, and/or additional agents of the present invention, whether labeled or unlabeled, as may be used to prepare a standard curve for a detection assay. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the phage-nanoparticle assembly or phage-nanoparticle assembly components, additional agents, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the kit components are placed, preferably, suitably allocated. The kits may also comprise a second container means for containing a sterile, pharmaceutically acceptable buffer and/or other diluent.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Nanoparticle Assembly

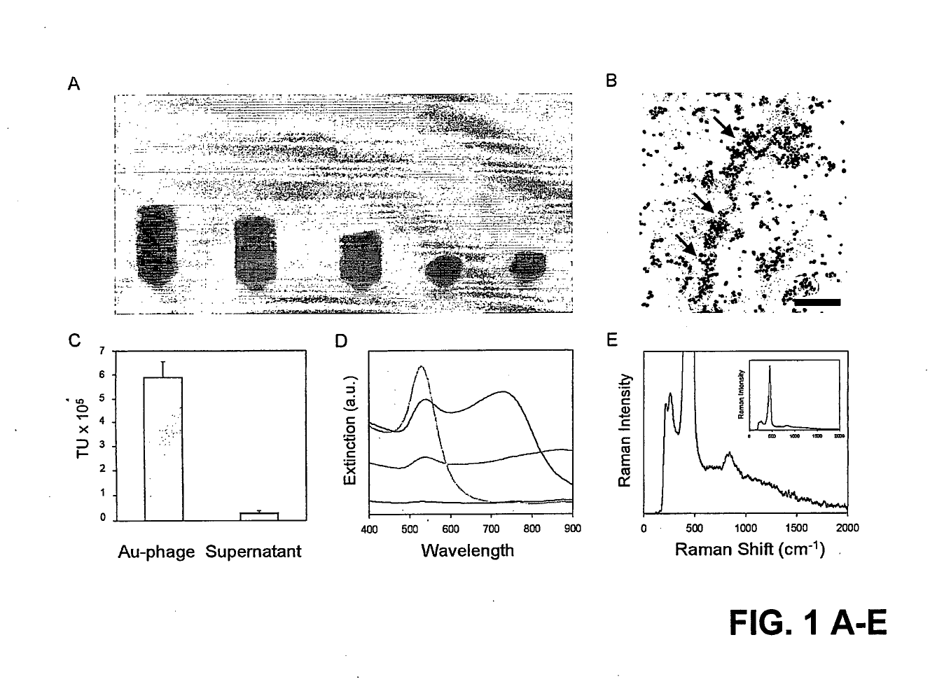

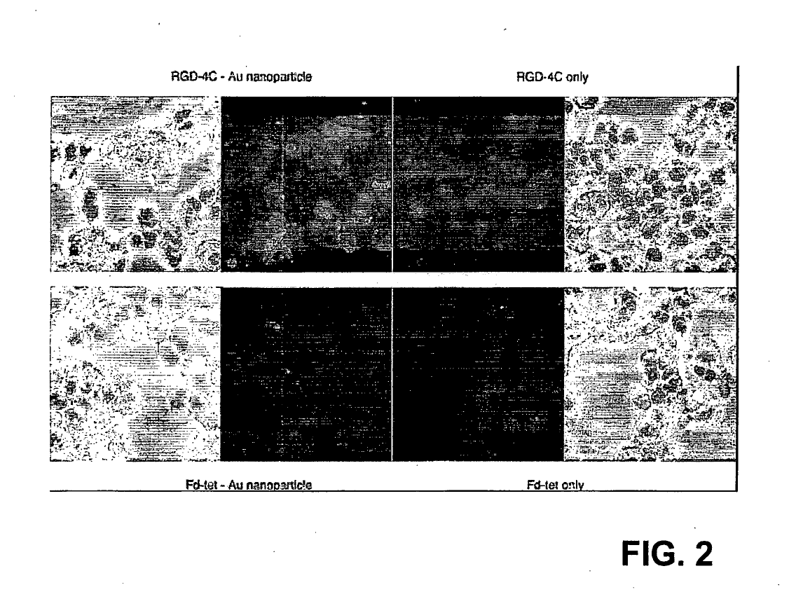

To study spontaneous assembly without modifying the phage capsid, the Inventors designed and validated an alternative approach for phage-based nano-assembly that does not rely on genetic manipulation or complex conjugation chemistry. The inventors show, for example, self-assembly direct-assembly of gold (Au) nanoparticles onto phage templates by simply manipulating solution properties (FIG. 1A-1E). Morphologically stable and distinct. networks (FIG. 1A) of self-assembled (FIG. 1B), biologically active (FIG. 1C) Au-phage assemblies were generated. Moreover, varying conditions altered the mechanical (FIG. 1A) and optical properties of the assemblies (FIG. 1D); this result not only supports the assembly of Au nanoparticles onto phage without covalent bonding but also strongly suggests the electrostatic nature of the Au-phage interaction (Shipway et al., 2000). Indeed, the inventors found the surface plasmon (SP) absorption wavelengths of the Au-phage assemblies can be tuned by changing the phage input in the presence of salts (FIG. 1D). The shift in the SP absorption to longer wavelengths indicates Au particle aggregation: higher phage input stabilized the Au nanoparticle assemblies by preventing salt-induced aggregation (FIG. 1D), while aggregation and precipitation did occur in the absence of phage (FIG. 1D). Finally, the Inventors detected the Au interaction with the phage surface by using surface enhanced Raman scattering (SERS) spectroscopy (Kneipp et al., 1999) (FIG. 1E), supporting Au-phage self-assembly. Taken together, these data show that controlled and spontaneous synthesis of Au-phage assemblies can be reproducibly accomplished without genetic manipulation of the phage template.

### Example 2

### Materials and Methods

Screening and Characterization of Targeting Peptides. The corresponding molecular addresses in target organ of interest will be identified, for example using the BRASIL method. In vivo and in vitro assays using individual phage will be performed in order to characterize the properties of each targeting peptide. (i) Lung targeting—endothelial cells recovered from lung vessels were used as the source of material for the selection of lung targeting peptides. The BRASIL method (Giordano et al., 2001) is used to obtain and evaluate lung homing properties of each phage individually. In vivo homing assays were used to evaluate the homing properties of selected peptides. (ii) Samples from endarterectomy procedure are used for the selection of peptides that home to atherosclerotic lesions. Homing properties of individual phage clones to atherosclerotic lesions are assessed by phage binding assay on human atherosclerotic plaques, and in animal models (LDL receptor, ApoE). (Note that other organs and tissues may be used to identify other targeted phage.) Tissue sections from several human organs are used for these studies. Phage binding is evaluated by using an anti-M13 antibody. Once a panel of suitable peptides is compiled, these probes are studied directly in human subjects to evaluate specificity (endarterectomy samples). Probes that do not accumulate in normal control organs are prioritized for targeting and receptor identification.

High-throughput analysis of selected peptides Statistical analysis of recovered peptide sequences by the MDACC Department of Biostatistics to determine homing motifs will be performed. The inventors have developed a character pattern recognition program to automate analysis of the CX₇C peptide sequences derived from high-throughput phage screenings (Arap et al., 1998). The program uses SAS (version 8, SAS Institute) and Perl (version 5.0) to conduct exhaustive residue sequence count in both directions and calculates relative frequencies of all tripeptide motifs encountered in the CX₇C peptides in each target tissue (or in the unselected library). Identified motifs are further used to search protein databases (NCBI) in search of candidate-receptors. Biochemical and molecular biology methods will be used to identify corresponding receptors, as described (Arap et al., 1998; Kolonin et al., 2004; Marchio et al., 2004; Pasqualini et al., 1997, 2000; Burg et al., 1999; Zurita et al., 2004; Koivunen et al., 1999).

Phage particle production Phage particles will be obtained as described in Pasqualiini et al., 2000. In brief, bacteria (E. coli K91kan) will be infected with phage for 20 minutes at RT, followed by culture in low-tetracycline media for the induction of the tet-resistance genes. Cells will then be culture overnight 37° C. at 280 rpm in LB media (supplemented with 20 μg/ml of tetracycline, 100 μg/ml of kanamycin). The next day, the bacteria will be centrifuged at 5,000 g for 10 min, and phage will be precipitated from the supernatant by PEG/NaCl, and resuspended in PBS. To obtain highly pure phage preparations, a second PEG precipitation will be performed. CsCl gradient separation results in the highest purity, but it is not always required. Phage titer and particle numbers will be calculated by bacterial infectivity assays, agarose gel and spectophotometric quantitation of phage as described (Pasqualini et al. 2000; Bonnycastle et al. 2000).

Au nanoparticle synthesis The synthesis of Au nanoparticles will be accomplished by applying the widely used Au auchloric reduction with citrate method (Bryant and Pemberton, 1991; Garrell and Pemberton, 1994; Lin et al., 1999; Ulman, 1996). The Au nanoparticle formation can be achieved by dissolving Au auchloric (Au(III)Cl, Sigma) in high purity boiling water, and then reducing the Au salt with specific amounts of trisodium citrate, which will be verified by a change in color from yellow (Au salt) to dark red (metallic Au colloids). The concentration of Au salt relative to the concentration of sodium citrate determines the rate of nucleation, which, consequently, determines the final size of Au nanoparticles. Purification of nanoparticles will be achieved by centrifugation or dialysis, depending on the size of the synthesized nanoparticles. To accomplish a 50 nm Au size the inventors used a 1:1 molar ratio of Au salt to citrate. The Au colloid size distribution obtained from this method varied approximately 10% around the mean particle size. The inventors will use transmission electron microscopy (TEM) and UV-Vis absorption spectroscopy to evaluate the Au nanoparticle size and size distribution. Because the optical properties of Au nanoparticles are a function of their size, UV-vis absorption spectroscopy will complement the TEM size measurements, where the wavelength of maximum absorption and the full-width at half maximum will allow estimation of particle size and size distribution, respectively.

For example, Insertless phage (fd-tet) and phage displaying the targeting peptide RGD-4C on the surface of its pIII protein, were amplified in host bacteria and purified (Barbas et al., 2001). The 44±9 nm Au nanoparticle solution, verified by TEM image analysis, was prepared following the common citrate-reduction (Handley, 1989) procedure (mass ratio of 0.8 sodium citrate: 1 Au(III) chloride). Au(III) chloride (99.99+%) was purchased from Sigma-Aldrich. Assembly of Au-phage complexes began with 8 serial dilutions of 10⁷ transducing units (T.U.) of phage in 200 μl nanopure water (>18.0 MΩ). An equal volume of Au solution (200 μl), first normalized to 4.2 a.u. (extinction measured at 528 nm), was added to each dilution, without mixing and allowed to stand for 12 h at room temperature (RT; here hydrogel formation takes place). Preparations were then mixed together in sequential order beginning with the least concentrated. Au-phage-imid networks were produced by mixing equal volumes of 10⁹ T.U./μl of phage and 1.0 M imidazole, followed by the addition of an equal volume of Au solution (4.2 a.u. extinction measured at 528 nm). Finally, the networks were purified by three consecutive centrifugation cycles (20,800 rcf for 28 min) in glass sterile tubes (BD Vacutainer, BD). Extinction of the Au-phage was measured at 528 nm and adjusted to between 1.2 and 1.5 a.u. by either diluting with water or concentrating by centrifugation. The fd-tet phage was used as a negative control to evaluate background under all the experimental conditions. Standard bacterial infection was used to determine phage titers as previously described (Dewey, 1997).

Au-phage assembly production The inventors contemplate producing at least three types of Au-phage assemblies: Au and phage only (Au-phage), Au and phage assembled in the presence of imidazole (Au-phage-imid) and Au and iron oxide (FeO) nanoparticles assembled with imidazole and phage (Au-phage-FeO, or SPION, Superparamagnetic Iron Oxyde Nanoparticles). The Au-phage are synthesized as a network of Au nanoparticles sparsely assembled with phage particles. The second type of scaffold, Au-phage-Imid, consists of a tightly assembled network of Au nanoparticles and phage due to the incorporation of imidazole. The third scaffold, Au-phage-FeO, include the assembly of FeO nanoparticles or Au-coated silica-FeO composites to be used as a magnetic resonance imaging (MRI) labels.

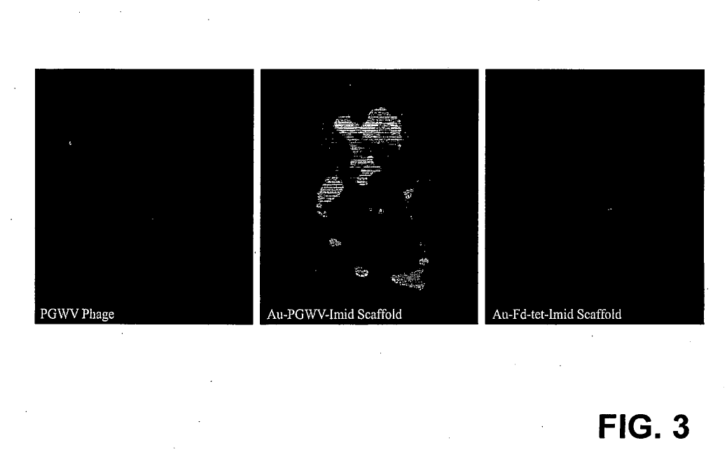

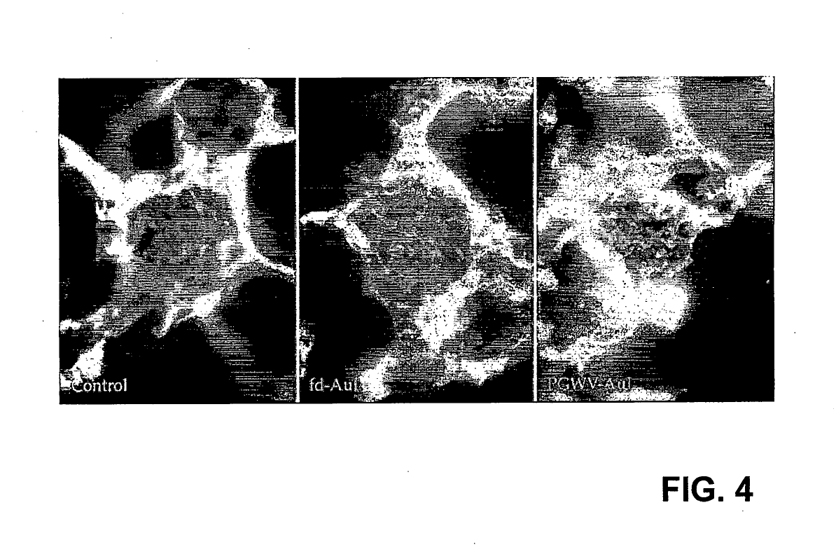

Au-phage assembly synthesis. The assemblies will be synthesized by the controlled mixture of Au nanoparticles and phage particles, where a series of 10 dilutions (1:2) of phage particles are dissolved in water with an equal volume of Au solution then added without mixing, and then the solution is allowed to stand overnight. After visible assembly has taken place, the 10 dilutions are mixed together from the least concentrated to the most. The phage input in the first dilution is approximately 1.0×10⁹ TU/μl (if stock phage solution input is lower than 1.0×10⁹ TU/μl, undiluted phage stock solution is used). The concentration of Au solutions is determined by its absorbance at 528 nm and adjusted to values between 1.2 and 1.5 absorption units (a.u.). If the Au concentration is outside of this range, it will be adjusted accordingly by either diluting or concentrating (by centrifugation) the Au solution. Because the structure and optical properties of the assemblies vary according to phage concentration (FIG. 3A), by using a range of concentrations the inventors assure that the different structures that contribute to the assemblies properties are present. This procedure will be optimized according to each application such as the type of nanoparticle used, the type of cell used, or if the studies are to be performed in vitro or in vivo. The inventors have reproducibly used assemblies produced with this procedure for in vitro detection of Kaposi sarcoma (KS) and lung endothelial cells (LECs). To maximize the consistency in detection measurements, the inventors will simultaneously synthesize two scaffolds: the control (pIII insertless phage, Fd-tet) and the targeting phage (pIII displayed targeting peptide) to be used as the control/sample pair during the targeting experiments. Assurance that the structural and optical properties are consistent between the control and targeting assemblies will be obtained by measuring the light absorption spectrum, which allows the concentration of the solutions to be adjusted relative to each other.

Synthesis of Au-phage-imid. The Au-phage-imid assemblies will be synthesized following the procedure above (Au-phage assembly synthesis), with the exception that the phage is diluted in 1M imidazole instead of water. As well as with Au-phage scaffolds, the inventors have reproducibly produced assemblies with this procedure for in vitro detection of KS and LECs, and for in vivo detection of LECs.

Synthesis of AU-phage SPION. The synthesis of Au-phage-SPION will be carried out by adjusting the two procedures listed above. Imidazole is well known for its capability to bind to metals, so FeO nanoparticles within the Au-phage-imid structure will be cross-linked with imidazole. The synthesis procedure will be adapted accordingly; for Au-phage-imid, FeO will be added simultaneously with Au nanoparticle to the dilutions of phage and imidazole, as the inventors expect the FeO to be incorporated within the Au-phage-imid structure.

Purification of assemblies. Purification will be accomplished by three consecutive centrifugation cycles (28 min. at 20,800 rcf each) using Teflon tubes. The assemblies are readily sediment by centrifugation, which makes this a relatively simple procedure. The supernatant of the last cycle is always saved and quantified along with the Au-phage assembly pellet, and the remaining supernatants are often discarded. However, if any new reagent is being used, all supernatants are tested for the final determination of the average number of phage per assembly.

Quantitation and characterization of assemblies. The characterization of the assemblies will be performed by using four approaches: (1) bacterial infection with purified Au-phage assemblies, (2) light absorption, (3) SERS, and (4) surface plasmon resonance (SPR). Bacterial infection is a functional assay for the quantitation of phage particles. The output of this measurement is the number of bacterial colonies (transducing units). Our studies show the average number of phage per assembly varies from 500 to 1000 phage equivalent per assembly. The inventors use the term phage equivalent to define the actual number of phage particles in solution; a method to accurately determine optimum stoicheometry of Au and phage particles is under optimization. Light absorption provides the level of aggregation of the Au nanoparticles indicated by the wavelength that the particles absorb and the amount of Au within the purified sample from the sample absorbance level. SERS measurements complement the absorption measurements, where the signal intensity is proportional to the concentration of Raman scattering molecules. Structural, chemical and optical characterization will be performed using transmission electron microscopy (TEM), light absorption spectroscopy, SERS spectroscopy/microscopy, fluorescence spectroscopy/microscopy, and nuclear magnetic resonance (NMR), for particles carrying FeO nanoparticles. The inventors will use TEM to analyze the structure of every scaffold used for biodetection. TEM capability is readily available in the MDAnderson Cancer Center EM Core Facility. Light absorption spectroscopy will be used to monitor the optical properties of Au nanoparticles within the assembly. The absorption spectra from the assembly indicate the arrangement of Au nanoparticles within the assembly, since the shift to a longer absorption wavelength and the intensity of this shift results from the proximity of the Au nanoparticles to each other and the level of aggregation. This is important because both SERS and photo-thermo response of the scaffolds are a function of the intensity and shape of the absorption spectrum. The inventor's laboratory is equipped with two high-throughput absorption plate readers (SpectraMax M2; Molecular Devices, Inc. & PowerWave 340; BioTek, Inc.) each housing a monochrometer as the light dispersing optical component, allowing the measurement of the absorption spectra. SERS spectroscopy will be first accomplished by using the R2001 Raman spectrometer (Raman Systems). This system is coupled to a fiber optic probe and is flexible enough to perform in vitro and in vivo measurements. SERS measurements will allow the probing of the chemical environment in the vicinity of the Au nanoparticles. Fluorescence measurements will be done to further explore the fluorescence enhancement provided by the Au nanoparticles using the high-throughput fluorescence plate reader SpectraMax M2 from Molecular Devices (Arap et al., 1998). A Biacore 2800 will be used for the SPR studies. This optical biosensor uses surface plasmon resonance (SPR) for real-time monitoring of molecular interactions, including affinity measurements, binding kinetics, concentration determinations, and binding specificity analyses. The SPR chip will be modified with anti-phage antibody to capture the assemblies from solution. The interaction of the immobilized antibody and phage in the scaffold will generate SPR signal changes, which will provide structural information about the scaffold, such as density and size.

Cell targeting assays. Cells will be plated on 8 chamber Culture Slides (BD Falcon) at a density of 1×10⁵ cells/cm² and grown overnight at 37° C. in Minimal Essential Medium (MEM) supplemented with 10% heat activated fetal bovine serum (FBS), 1% MEM-Non-Essential Amino Acids, 1% MEM Vitamin Solution, and 1% penicillin-streptomycin Glutamine (GIBCO). The next day each chamber will be blocked with MEM/28% FBS for 1 h at 37° C. Cell chambers will be incubated with phage (1.0×10⁹ TU) or Au-phage (˜1.0×10⁶ TU) for 12 hr at 37° C. in MEM 2% FBS. The negative control will be cells alone and Fd-tet phage with Au or Au and imidazole. Free phage and weakly surface bound phage will be removed by washing with a glycine buffer (50 mM glycine, 150 mM NaCl, pH 2.8) followed by washes with PBS (Ca²⁺ and Mg²⁺ free). Cells will be fixed with fresh 4% paraformaldehyde in PBS for 15 min. at room temperature (RT) and washed 3 times with PBS. The cells will be permeabilized with 0.2% Triton X-100 for 5 min at RT, washed 5× with PBS, blocked with PBS/1% BSA for 2 hours at RT and incubated with rabbit anti Fd-tet (Sigma) for 2 hrs at RT followed by 5 rinses with PBS/1% BSA. Finally, for fluorescence microscopy imaging, the cells will be incubated for 1 hr with a Cy3 labeled anti-rabbit IgG antibody (Jackson ImmunoResearch). DAPI containing cytoseal will be used during the slide mount preparation.

In vivo tissue ablation and SERS detection. The in vivo studies will be performed according to each application. The general procedure consists of administering the solution of assemblies intravenously (i.v), delivering laser light onto the targeted tissue, and then detecting the SERS signal. Volume, concentrations, time of circulation, and laser power exposure will be optimized by detecting assemblies and ablating tissues using postmortem mice and then adapted for each animal model and specific experimental question. The inventors will optimize the laser power exposure along with level of tissue damage and magnitude of SERS signal in vivo by probing the targeted tissues non-invasively or by opening the chest cavity. Laser power will be controlled through exposure time, laser intensity attenuation, and/or laser pulsing.

Hydrogel Biocompatibility and Cytotoxicity. Assembly of Au-phage hydrogel was achieved by adding 150 μl of Au nanoparticles (1.2 a.u for extinction at 528 nm) to 150 μl aqueous solution of RGD-4C displaying phage (0.14 a.u. for extinction at 270 nm) under sterile conditions in a 96-microwell plate. Hydrogels were allowed to form over night at 4° C. After hydrogel formation, solution was exchanged by removing the supernatant, while avoiding disturbing hydrogel structure, and then adding an equal volume of media (10% FCS in DMEM high glucose with sodium pyruvate, 2 mM glutamine, penicillin and streptomycin). C17.2 murine neural stem bells (NSC; 1.0×10⁴ cells/well) (Snyder et al., 1992) were added and allowed to grow for several days at 37° C. Photomicrographs were taken 12 h after cell addition.

TEM Imaging. Nickel mesh grids previously coated with Formvar® and evaporated with carbon were floated on drops of 0.1% poly-L-lysine (Sigma Diagnostics) on Parafilm for 5 min. Excess solution was removed from the grid by carefully touching the edge of the grid onto filter paper. The grids were not allowed to dry completely in any of the following steps. The grids were floated on drops of sample on Parafilm for 1 h. Excess fluid was removed as above and the grids were then floated on drops of 1% ammonium molybdate in 0.02% BSA in distilled water, pH 7.0 for 60 sec. Excess fluid was removed and the grids were allowed to dry overnight. TEM images were captured by a transmission electron microscope (JEOL JEM-1010) fitted with an AMT Advantage digital CCD camera system. Au nanoparticle size (44±9 nm) was determined by averaging particle sizes within representative TEM fields. The fractal dimension (Df) analysis of the two-dimensional TEM images was performed with the box counting method (ImageJ v.1.33 software). Df measurements were averaged from 10 separate TEM images of three different samples.

Cell Targeting and Peptide Inhibition Assay. B-16 malignant melanoma cells were seeded on 16 chamber culture slides (BD Falcon) at a density of 5×10⁴ cells/well and grown overnight at 37° C. in RPMI media (Gibco-BRL) containing 10% FBS, antibiotics and 1% L-glutamine. The next day, each well was blocked with RPMI 28% FBS for 1 h at 37° C. and incubated with different concentrations of RGD-4C synthetic peptide (from 10⁻³ nM to 10⁻¹⁰ nM) for 28 min. Suspended and purified Au-phage (10⁷ T.U.) solutions were then added to the wells. After 12 h, fluorescence phage staining was performed as described below (Fluorescence Imaging).

Fluorescence Imaging. Following the cell targeting assay, cells were washed and fixed with PBS containing 4% paraformaldehyde. The cells were then permeabilized with 0.2% Triton X-100, washed, blocked with PBS containing 1% BSA cells were incubated with rabbit anti-fd bacteriophage antibody (Sigma) for 2 hr at RT, followed by a 1 h incubation with Cy3-labeled anti-rabbit IgG antibody (Jackson ImmunoResearch). Finally, cells were again fixed with PBS containing 4% paraformaldehyde and mounted in the presence of DAPI (nuclear staining dye, Vectashield, Vector Laboratories). Images were acquired with an Olympus fluorescence microscope equipped with an Hg lamp and a band-pass excitation filter (528 nm to 555 nm) in the fluorescence excitation path and a long-pass dichroic filter (570 nm) and long pass filter (590 nm) in the emission path.

Confocal Fluorescence Imaging. Following the cell targeting assay, cells were incubated with rabbit anti-fd bacteriophage antibody and mouse anti-β₁ integrin antibody in PBS 1% BSA for 2 h at RT, followed by a 1 h incubation with Cy3-labeled anti-rabbit IgG antibody and Cy5 labeled anti-mouse IgG antibody diluted in PBS containing 1% BSA (Jackson ImmunoResearch). SYTOX Green nucleic acid stain (Molecular Probes) was then incubated with the cells for 10 min. Confocal images were acquired with a Zeiss LSM510 laser scanning confocal microscope by using krypton-argon and helium-neon lasers. Image analysis and stack projections were created with the Zeiss LSM software package (v3.2).

Darkfield Imaging. Dark field images were acquired prior to the permeabilization step and antibody incubations described in the cell targeting assay procedure with an Olympus fluorescence microscope equipped with a darkfield condenser.

SERS Detection of Cells. KS1767 cells (2×10⁵) were incubated in 1.0 ml of MEM containing 2% FBS with phage (10⁹ T.U.) or Au-phage (10⁶ T.U.) for 18 hr at 37° C. Negative controls included cells alone and fd-tet phage, Au-fd-tet, Au-fd-tet-imid and Au only. Each tube was washed with a glycine buffer (50 mM Glycine and 150 mM NaCl at pH 2.8) followed by several PBS washes. Cells were then counted and normalized to the lowest cell count. SERS measurements of suspended cells were gathered by using an R2001 Raman spectrometer (Ocean Optics, Inc.) equipped with a fiber-optic probe to deliver 785 nm laser light and to collect the Raman-scattered light.

### Example 3

### Design, Synthesis, and Characterization of Au-Phage Networks

Spontaneous assembly of gold nanoparticles onto phage was contemplated to occur without genetic modification of the pVIII major capsid proteins or complex conjugation chemistry (FIG. 21). In order to test that hypothesis, biologically active networks of directly assembled Au-phage complexes were generated by optimizing the phage concentration required to convert Au colloidal solutions into hydrogels, which is the precursor for generating network suspension (FIG. 21B). The biocompatibility and cytotoxicity were tested of these networks by showing that NSCs widely infiltrate the Au-phage hydrogel network structure indicated by the stretched Au-phage fibers (FIG. 21C-21D) and continue to proliferate (data not shown). Next, it was found that the surface plasmon (SP) absorption wavelengths of the Au-phage complexes can be modulated by changes in phage input and the presence of imidazole. Transmission electron microscopy (TEM), elastic light scattering, visible/near-infrared (NIR) absorption and NIR surface-enhanced Raman scattering (NIR-SERS) confirmed Au-phage assembly. Data on these findings are presented in the following paragraphs.

Two strategies were used to generate biologically active networks: with or without the metal binding molecule imidazole (FIG. 21A) (Souza and Miller, 2001; Dewey, 1997). The different assemblies yielded distinct network organizations, as revealed by TEM (FIG. 21E). Au nanoparticles appear as black dots connecting long white filamentous phage structures. Once assembled, the phage in these networks still maintained their ability to infect bacteria (FIG. 21F) and showed distinctive physical characteristics, such as fractal structure and NIR optical properties. Because fractal patterns are often observed in naturally occurring assembly processes (Dewey, 1997; Mandelbrot, 1982), it was evaluated whether the spontaneously assembled structures would show fractal traits and if this feature could distinguish the structural differences between the networks. Fractal dimension (Df) analysis of the 2D TEM images (Au-phage 1.32±0.12 and Au-phage-imid 1.78±0.14; t-test, p<0.0001) correlated with the apparent aggregation of Au nanoparticles within each network. The lower Df for Au-phage indicated looser, more dispersed structures observed by TEM (FIG. 21E, upper panel); in comparison, the higher Df for the Au-phage-imid reflected the denser networks as a result of imidazole-induced aggregation (Au-imid) of Au nanoparticles (FIG. 21E, lower panel). Consistently, angle-dependent elastic light scattering fractal dimension analysis (based on Rayleigh-Debye-Gans scattering theory) (Avnir et al., 1984; Farias et al., 1996) revealed the same Df trend for the networks in solution (data not shown) as that obtained from the TEM image analysis. The characteristic high-surface area of fractal networks (West et al., 1999) can improve accessibility to binding sites, which is a central feature for fabricating cell-targeting systems.

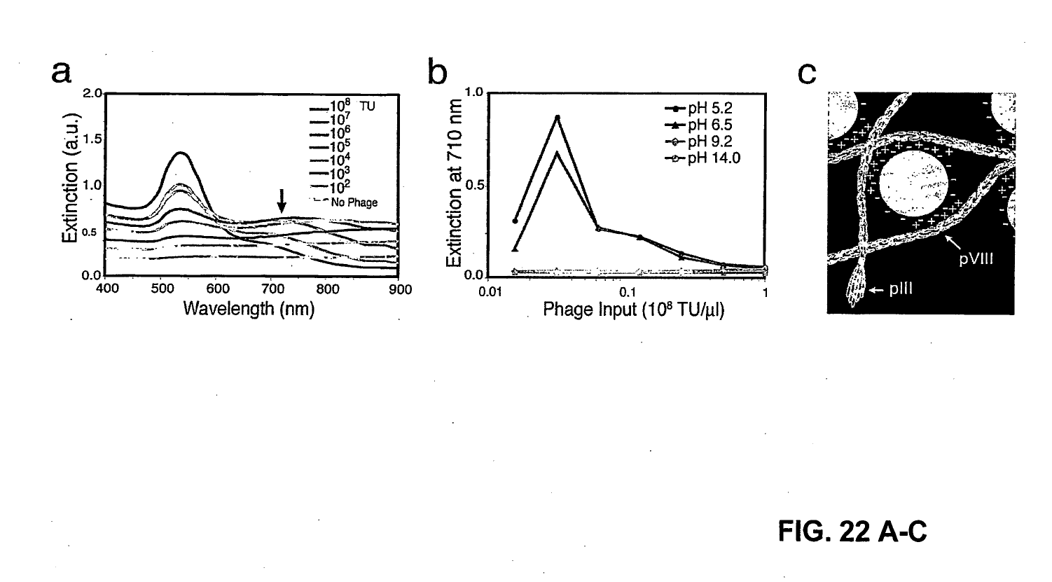

Next, the network formation was evaluated by monitoring the shift in SP absorption into the NIR spectral region when phage concentration was increased (FIG. 22A, spectra red shift indicated with an arrow; and FIG. 22B). The red shift, which generally occurs when the distance between Au nanoparticles (dAu) is less than the average particle diameter (2rAu; dAu<2rAu), is usually a good diagnostic for Au—Au interactions due to particle agglomeration (Mirkin et al., 1996; Shipway et al., 2000; Weisbecker et at., 1996)

It is contemplated that the native pVIII major capsid proteins function as the binding sites for the Au-phage network assembly (FIG. 21A). Given the absence of typical metal binding amino acid residues (such as Cys and His) on the pVIII protein, the direct assembly of Au nanoparticles onto phage should be largely directed by electrostatic interactions (Mirkin et al., 1996; Shipway et al., 2003; Dujardin et al., 2003; Purdy and Fraden, 2004; Marvin, 1998; Tang et al., 2002; Zimmermann et al., 1986). It is well known that gold nanoparticles can be made to agglomerate by varying solution ionic conditions (specifically, agglomeration shows a dependence on ionic strength). In solution, gold nanoparticles are coated with a layer of adsorbed citrate anions (citrate from Au nanoparticle synthesis procedure) (Mirkin et al., 1996; Shipway et al., 2000; Weisbecker et al., 1996; Handley, 1989). Attraction between like-charged particles can occur due to correlated fluctuations in the surrounding ion clouds. Thus, the presence of ions can be used to mediate the agglomeration. For example, gold nanoparticles agglomerate in the presence of salt as indicated by a broadening and shift to longer wavelength in the surface plasmon absorption peak (FIG. 22A, no phage, green spectrum). It has been reported that phage particles (both fd and M13) also act as polyanionic particles in solution with several negative surface charges associated with each of the ≈2700 copies of the major capsid protein (fd is more anionic than M13 due to the replacement of Asn12 with Asp12) (Purdy and Fraden, 2004; Tang et al., 2002; Zimmermann et al., 1986; Marvin, 1998). Further, bundles of phage form from like-charge attraction (Purdy and Fraden, 2004; Tang et al., 2002; Zimmermann et al., 1986; Marvin, 1998) and, analogous to the mediation of gold nanoparticle agglomeration, solubilization of such bundles is dependent on solution ionic strength. Gold agglomeration was induced by 0.25 M NaCl (FIG. 22A; no phage) could be minimized by Au-phage interactions (indicated by the small red shift) when phage input increased. This suggests a similar physical interpretation for binding in the mixed phage/gold systems as that found in the gold-gold and phage-phage binding. These findings indicate a greater stability of the gold-phage networks in the presence of salt.

To further explore the role of electrostatics in the assembly mechanism, network formation was monitored at a series of solution pH (FIG. 22B). Under all tested conditions phage have an overall negative charge, but we observed formation of the Au-phage networks only at pH well below the calculated pI (9.4) of the individual pVIII proteins (Gasteiger et al., 2005). For solutions where networks did form (as indicated by the extinction at 710 nm attributable to gold aggregation), lower pH led to increased extinction. These results imply that the pVIII positive charge at pH≦7 mediates the mechanism of assembly through opposite charge interaction (FIG. 22C) between the citrate-adsorbed Au nanoparticles and the thin and long phage surface (6 nm×1,000 nm) (Purdy and Fraden, 2004; Tang et al., 2002; Zimmermann et al., 1986; Marvin, 1998). Finally, the data in FIG. 22B show that at low phage input level, the aggregation falls off due to titration of phage binding sites in the solution.

Next, the optical properties of the purified Au-phage and Au-phage-imid network solutions were compared. The more compact Au-phage-imid complexes had a relatively larger red shift in the extinction spectrum with increased absorptivity in the NIR wavelength region (700-900 nm; FIG. 23A) (Weisbecker et al., 1996; Elghanian et al., 1997). In order to support the assertion that NIR photons were also being absorbed instead of only scattered by the larger agglomerates, the temperature change was measured for the two network solutions as a direct function of illumination-time using NIR incident laser light (785 nm; FIG. 23B). There was a substantial temperature change for both the Au-phage and Au-phage-imid networks from the efficient photon-to-heat conversion, which demonstrates that the NIR photons are being absorbed as a result of the SP absorption red shift.

Finally, NIR-SERS spectroscopy was used to characterize the interactions among Au, phage and imidazole (FIG. 23C and Table 1). Differences between the spectra of Au-phage and Au-phage-imid seem to arise from the distinct chemical environments in the vicinity of the Au nanoparticles. Signature bands for phage and Au interaction emerged by identifying common attributes when comparing NIR-SERS spectra intrinsic to each of the networks (Aubrey and Thomas, 1991; Overman and Thomas, 1999) and controls. First, from an analysis of control experiments without phage (Au-imid, FIG. 23C orange curve), the 1028 cm−1 band is a SERS feature only detected in the networks spectra (Aubrey and Thomas, 1991; Overman and Thomas, 1999; Schwartzberg et al., 2004) and could be assigned to Tyr, Phe and/or Met (pVIII major capsid protein). Second, the mode at 1445 cm−1 is likely attributed to Trp and/or Met (Aubrey and Thomas, 1991; Overman and Thomas, 1999; Schwartzberg et al., 2004). A third peak was only seen in the Au-phage spectrum, the broad and low intensity peak centered at 840 cm−1, which has been assigned to Tyr residues present within the pVIII (Aubrey and Thomas, 1991; Overman and Thomas, 1999). Several other peaks (750, 840, 954, 1109, 1169, and 1268 cm−1) present in the Au-phage-imid spectrum have been observed in systems were imidazole is adsorbed onto gold Holze, 1993) or silver Cao et al., 2003) electrodes. Additional peaks in the Au-phage spectrum might be attributable to exposed residues on the major capsid. For example, strong SERS signals near 1147 and 1281 cm−1 have been observed in systems of both lysine and methionine with Au nanoparticles (the last three amino acids of the exposed N-terminal end of the pVIII capsid are Lys-Lys-Met) (Schwartzberg et al., 2004).

TABLE 1
Assignment of Raman bands and amino acids in the SERS spectra shown in FIG. 3c.

| Au-phage | Au-phage-imid | Au-imid | Imidazole | Side Chain or | Vibrational | |
|---|---|---|---|---|---|---|
| (cm⁻¹) | (cm⁻¹) | (cm⁻¹) | (cm⁻¹) | Imidazole Designation | Assignment* | Ref |
| | 670 | 650 | | Imidazole | γ_{ring} (628 cm⁻¹, Raman) | (38, 39) |
| | 750 | 764 | | Imidazole | γ_{ring} (743 cm⁻¹, SERS) | (38) |
| | 840 | 838 | | Imidazole | γ_{ring} (832 cm⁻¹, SERS) | (39) |
| 854 | | | | Tyr, Ile and/or Met | γ_{ring} (853 cm⁻¹, Raman), γ(CC) (874 cm⁻¹, | (36, 37) |
| | | | | | Raman and SERS) | |
| | 954 | 954 | 931 | Imidazole | γ(NH) + δ_{ring} (950 cm⁻¹, SERS) | (38, 39) |
| 1030 | 1030 | | | Tyr, Phe and/or Met | γ(CH) (1033 cm⁻¹, SERS), ν(CC, CN, CO) | (35) |
| | | | | | (1031 cm⁻¹, Raman) | |
| | 1109 | 1109 | | Imidazole | δ(CH) + ν_{ring} (1097 cm⁻¹, SERS) | (38, 39) |
| 1147 | | | | Met and Lys | ν(CC) (1158 cm⁻¹, Raman) | (35) |
| | 1169 | 1169 | 1160 | Imidazole | δ_{ring} (1164 cm⁻¹, SERS) | (38, 39) |
| | 1268 | 1268 | 1263 | Imidazole | δ(CH) (1265 cm⁻¹, SERS) | (38, 39) |
| 1301 | | | | Amide III, Tyr, Ala, | δ(CH) and ν(CC)(1300 cm⁻¹, Raman) | (35-37) |
| | | | | Lys, Ile, Val, Ser, | | |
| | | | | Gly, Trp and pVIII | | |
| | | | | main chain | | |
| | 1328 | 1328 | | Imidazole | ν_{ring} (1329 cm⁻¹, SERS) | (38, 39) |
| 1445 | 1436 | 1415 | 1436 | Trp and Imidazole | ν_{ring} (1449 cm⁻¹, Raman) | (38, 39) |

*δ = in-plane bending; γ = out-of-plane bending; ν = stretching (37)

### Example 4

### Targeted and Fluorescence Enhanced Cell Detection

To evaluate whether Au-phage-based networks could be efficiently used to study peptide ligand binding to receptors on the cell surface as well as receptor-mediated properties such as phage internalization, immunofluorescence-staining assays were performed with antibodies directed to the phage capsid. Melanoma cells were chosen because they express high levels of αv integrins (Albelda et al., 1990) the cell surface receptor for a well characterized phage displaying the peptide CDCRGDCFC (termed RGD-4C) (Arap et al., 1998). Here it is shown that the targeting and receptor-mediated internalization capabilities of the RGD-4C peptide remained intact within the Au-RGD-4C networks. Accordingly, internalization was inhibited (Chen et al., 2004) in a dose-dependent manner when cells were pre-incubated with the RGD-4C synthetic peptide prior to incubation with Au-RGD-4C networks. The likely synergy between the receptor-mediated phage internalization and electromagnetically-induced surface enhancement of the Au nanoparticles (Kneipp et al., 1999) resulted in an increase in the enhanced fluorescence for the targeted Au-RGD-4C networks relative to those observed for the RGD-4C phage alone. Negative controls show only background signal.

### Example 5

### Confocal Fluorescence Image Analysis

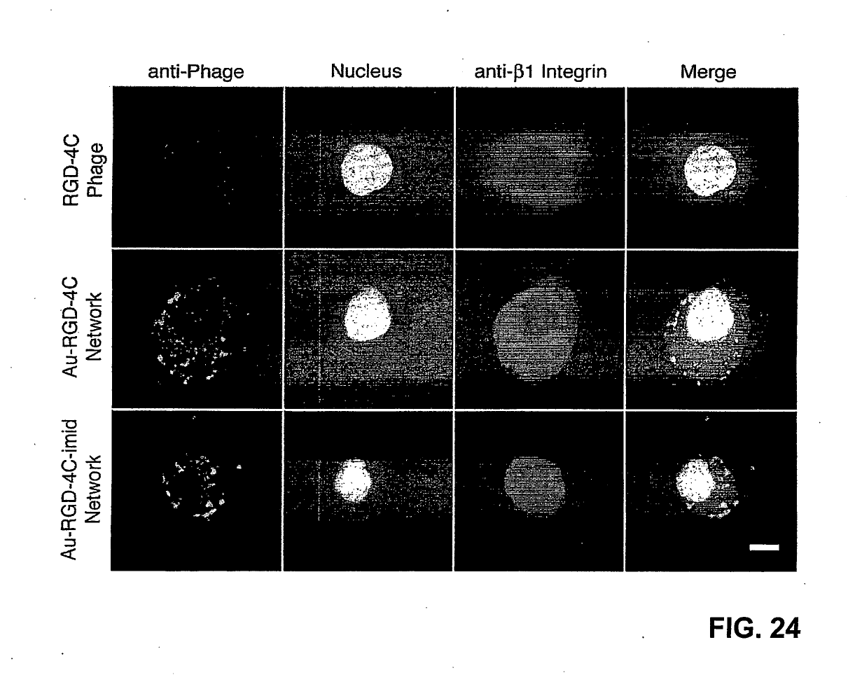

It is contemplated that, when examined by confocal microscopy, Au-phage networks could serve as sensitive reporters to localize and evaluate ligand binding and receptor-mediated internalization events (FIG. 24). It appears that differences in the structure of the targeting networks result in distinct kinetics of the internalization event that follows ligand-receptor binding. By incorporating imidazole into the nano-architecture of the networks, we were able to influence the localization of the Au-phage networks to either the cell surface or cytoplasm. More compact networks with a higher Df (Au-RGD-4C-imid) preferentially localized at the cell surface, while those with a lower Df (Au-RGD-4C) were internalized (FIG. 24). Dynamic examination of confocal image stacks from different cell planes is consistent with this interpretation (data not shown). These results lead us to hypothesize the existence of an aggregate morphology-dependence (as measured by Df) for receptor-mediated internalization, in which network changes from more to less compact structures might favor cell internalization over surface binding. These findings suggest that the control of network morphology through titration with a nanoparticle complexing agent such as imidazole may be used to modulate their ligand-directed cell targeting ability to match a desired application.

### Example 6

### Elastic Light Scattering and SERS Detection of Cells with Targeted Networks

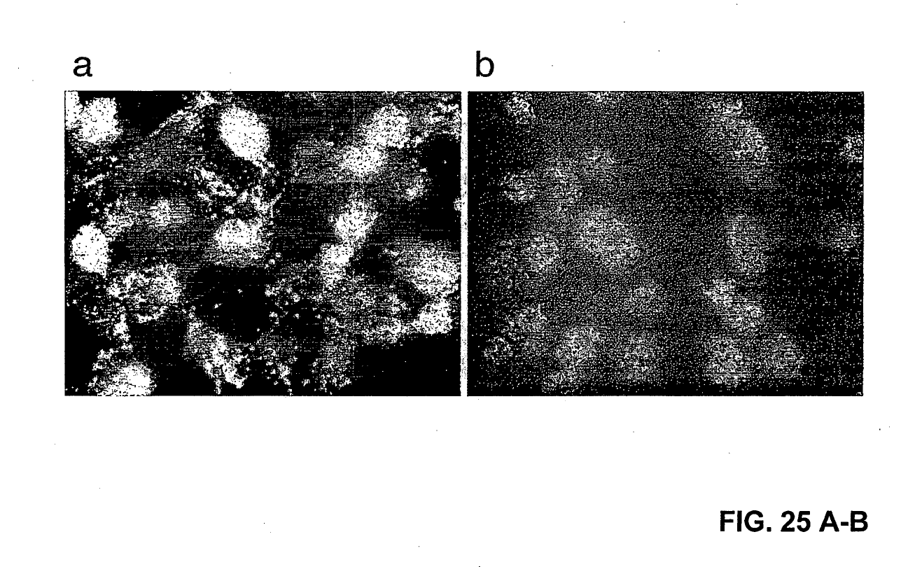

To extend the light scattering attributes of the networks into analytical applications, darkfield microscopy was used to generate a single-step, fast and sensitive imaging system that does not require staining (FIG. 25). Darkfield microscopy detects scattered light, and the large scattering cross-section of the Au nanoparticles (Souza and Miller, 2001; Yguerabide and Yguerabide, 1998a; Yguerabide and Yguerabide, 1998b) makes them ideal contrast agents. The Au-RGD-4C targeted networks (FIG. 25A) when compared to RDG-4C phage without Au (data not shown) or networks formed by untargeted nanoparticles (FIG. 25B) showed markedly increased signal.

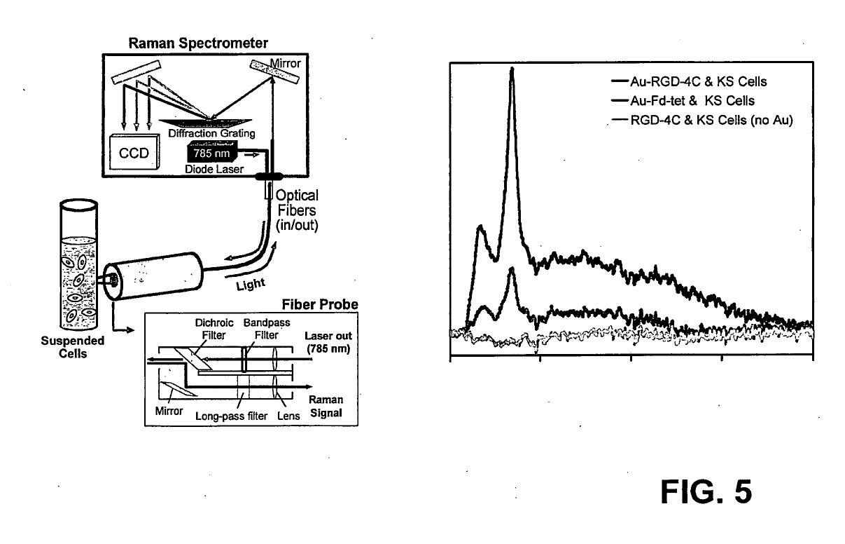

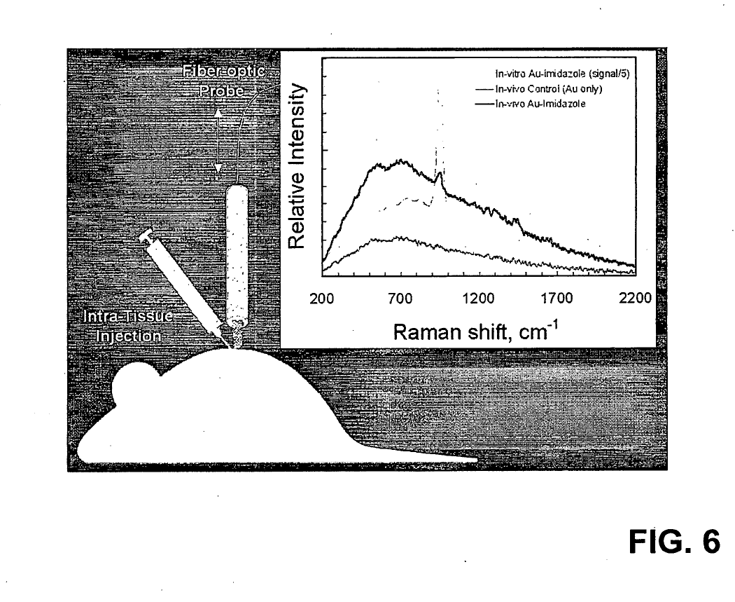

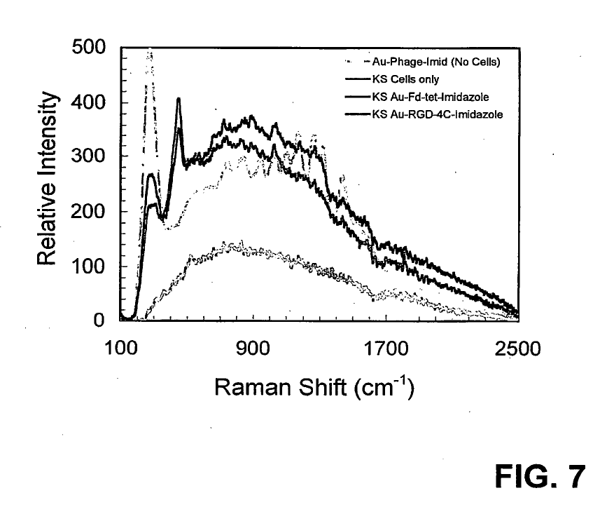

The potential of Au-phage-imid networks as cell nanosensors was assessed by integrating their unique Raman spectrum to their biological activity (see FIG. 5). The SERS spectra of suspended cells incubated with Au-phage-imid were measured by using a fiber-optic probe to deliver 785 nm laser light and to collect the Raman signal into a spectrometer (see FIG. 5). The NIR-SERS spectra of mammalian cells incubated with Au-phage-imid networks were obtained (see FIG. 5). The high signal intensity (blue spectrum) directly correlated with the level of cell binding and internalization by the networks carrying phage directed at αᵥ integrin receptors on the surface of target cells. Cells treated with RGD-4C phage alone (red spectrum) could not be differentiated from untreated cells. Background only (green spectrum) was observed with fd phage. The sensitive detection and distinct SERS spectra for the cells treated with Au-phage-imid showed that phage, Au nanoparticles and imidazole (or imidazole-like molecules) can be combined to form distinct Raman signal signatures as reporters for receptor-mediated targeting in cells or tissues. Peak intensity differences in SERS spectra between the cell-free (FIG. 23C) and bound Au-phage-imid (FIG. 23C) are likely the result of cell binding and internalization events. Because of the very high chemical selectivity and sensitivity of NIR-SERS spectroscopy (Cao et al., 2002) the Raman labeling strategy described here can provide SERS-based labels for high-throughput molecular and biological detection schemes.

### REFERENCES

U.S. Pat. No. 4,659,774
U.S. Pat. No. 4,682,195
U.S. Pat. No. 4,683,202
U.S. Pat. No. 4,816,571
U.S. Pat. No. 4,959,463
U.S. Pat. No. 5,141,813
U.S. Pat. No. 5,223,409
U.S. Pat. No. 5,223,409
U.S. Pat. No. 5,264,566
U.S. Pat. No. 5,286,403
U.S. Pat. No. 5,403,484
U.S. Pat. No. 5,427,908
U.S. Pat. No. 5,427,908
U.S. Pat. No. 5,428,148
U.S. Pat. No. 5,492,807
U.S. Pat. No. 5,516,637
U.S. Pat. No. 5,554,744
U.S. Pat. No. 5,571,698
U.S. Pat. No. 5,574,146
U.S. Pat. No. 5,580,717
U.S. Pat. No. 5,602,244
U.S. Pat. No. 5,622,699
U.S. Pat. No. 5,645,897
U.S. Pat. No. 5,658,727
U.S. Pat. No. 5,670,312
U.S. Pat. No. 5,698,426
U.S. Pat. No. 5,705,610
U.S. Pat. No. 5,705,629
U.S. Pat. No. 5,733,743
U.S. Pat. No. 5,750,753
U.S. Pat. No. 5,780,225
U.S. Pat. No. 5,821,047
U.S. Pat. No. 5,840,841
U.S. Pat. No. 5,969,108
U.S. Pat. No. 6,002,471
U.S. Pat. No. 6,068,829
U.S. Pat. No. 6,174,677
U.S. Patent Appln. 20028152578
U.S. Patent Appln. 20040048243
U.S. Patent Appln. 20040077844
U.S. Prov. Patent Appln. 20040110208
Albelda et al., Cancer Res., 50:6757-6764, 1990.
Ames et al., J. Immunol. Methods, 184:177-186, 1995.
Arap et al., Curr. Opin. Oncol., 10:560-565, 1998.
Arap et al., Science, 279:377-380, 1998.
Aubrey and Thomas, Biophys. J., 60:1337-1349, 1991.
Averitt et al., Phys. Rev. Lett., 78:4217-4220, 1997.
Avnir et al., Nature, 288:261-263, 1984.
Baichwal and Sugden, In: Gene Transfer, Kucherlapati (Ed.), NY, Plenum Press, 117-148, 1986.
Barbas et al., In: Phage display, A Laboratory Manual, NY: Cold Spring Harbor, 2001.
Barrow and Soothill, Trends Microbiol., 5, 268-271, 1997.
Bonnycastle et al., In: Phage Display, A Laboratory Manual. Scott (Ed.), NY: Cold Spring Harbor Laboratory Press, 22:21-24, 2000.
Brinkman et al., J. Immunol. Methods, 182:41-50, 1995.
Bryant and Pemberton, J. Am. Chem. Soc., 113:8284-8293, 1991.
Burg et al., Cancer Res., 59:2869-2874, 1999.
Burton et al., Advances in Immunology, 57:191-280, 1994.
Cao et al., J. Phys. Chem. B, 107:769-777, 2003.
Cao et al., Science, 297:1536-1540, 2002.
Carter and Flotte, Curr. Top Microbiol. Immunol., 218:119-144, 1996.
Chen and Okayama, Mol. Cell Biol., 7(8):2745-2752, 1987.
Chen et al., Chem. Biol., 11:1081-1091, 2004.
Coupar et al., Gene, 68:1-10, 1988.
Crow et al., Br. J. Cancer, 89:106-108, 2003.
Desmettre et al., Surv. Ophthalmol., 45:15-27, 2000.
Dewey, In: Fractals in Molecular Biophysics, Oxford University Press, New York, 1997.
Dujardin et al., Nano. Lett., 3:413-417, 2003.
Dutta and Hofmann, In: Self organization of colloidal nanoparticles, Encyclopedia of Nanoscience and Nanotechnology, Nalwa (Ed.), American Scientific Publishers, Stevenson Ranch, Calif., 1-23, 2003.
Elghanian et al., Science, 277:1078-1081, 1997.
EP 266 032
Farias et al., Appl. Opt., 35:6560, 1996.
Fechheimer, et al., Proc Natl. Acad. Sci. USA, 84:8463-8467, 1987.
Ferrari et al., J. Virol., 70(5):3227-3234, 1996.
Flotte et al., Proc. Natl. Acad. Sci. USA, 90(22):10613-10617, 1993.
Fraley et al., Proc. Natl. Acad. Sci. USA, 76:3348-3352, 1979.
Froehler et al., Nucleic Acids Res., 14(13):5399-5407, 1986.
Garrell and Pemberton, In: Fundamentals and applications of surface raman spectroscopy, Deerfield Beach, Fla., VCH Publishers, 1994.
Gasteiger et al., In: The Proteomics Protocols Handbook, Walker (Ed.), Human Press, Totowa, 571-608, 2005.
Gatti and Rivasi, Biomaterials, 23:2381-2387, 2002.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Giordano et al., Nat. Med., 7:1249-1253, 2001.
Goodman et al., Blood, 84(5):1492-1500, 1994.
Gopal, Mol. Cell Biol., 5:1188-1190, 1985.
Graham and Van Der Eb, Virology, 52:456-467, 1973.
Grubisha et al., Anal. Chem., 75:5936-5943, 2003.
Handley, In: Colloidal Gold: Principles, Methods, and Applications, Hayat (Ed.), Academic Press, San Diego, 1:23-27, 1989.
Harland and Weintraub, J. Cell Biol., 101(3):1094-1099, 1985.
Harlow and Lane, In: Antibodies: A Laboratory Manual, Cold Spring Harbor Laboratory, Cold Spring Harbor, N.Y., 346-348, 1988.
Hartschuh et al., Science, 281:1354-1356, 2003.
Hirsch et al., Anal. Chem., 75:2377-2381, 2003.
Holze, Electrochim. Acta, 38:947-956, 1993.
Kaneda et al., Science, 243:375-378, 1989.
Kaplitt et al., Nat. Genet., 8(2):148-154, 1994.
Kato et al, J. Biol. Chem., 266:3361-3364, 1991.
Kessler et al., Proc. Natl. Acad. Sci. USA, 93(24):14082-14087, 1996.
Kettleborough et al., Eur. J. Immunol., 24:952-958, 1994.
Klein et al., Nature, 327:70-73, 1987.
Kneipp et al., Chem. Rev., 99:2957-2976, 1999.
Koeberl et al., Proc. Natl. Acad. Sci. USA, 94(4):1426-1431, 1997.
Koivunen et al., Nat. Biotechnol., 17:768-774, 1999.
Kolonin et al., Nat. Med., 6:625-632, 2004.
Lee et al., Science, 296:892-895, 2002.
Lin et al., Bioconjug. Chem., 13:605-610, 2002.
Lin et al., J. Phys. Chem., B103:5488-5492, 1999.
Macejak and Sarnow, Nature, 353:90-94, 1991.
Mandelbrot, In: The Fractal Geometry of Nature, Freeman, San Francisco, 1982.
Mao et al., Proc Natl. Acad. Sci. USA, 100:6946-6951, 2003.
Mao et al., Science, 283:213-217, 2004.
Marchio et al., Cancer Cell, 5:151-162, 2004.
Marvin, Curr. Opin. Chem. Biol., 8:150-158, 1998.
Marvin, Curr. Opin. Struct. Biol., 8:150-158, 1998.
McCown et al., Brain Res, 713(1-2):99-107, 1996.
Mirkin et al., Nature, 382:607-609, 1996.
Mizukami et al., Virology, 217(1):124-130, 1996.
Mordon et al., Lasers Surg. Med., 18:265-270, 1996.
Mordon et al., Lasers Surg. Med., 20:131-141, 1997b.
Mordon et al., Lasers Surg. Med., 21:365-373, 1997a.
Mordon et al., Microvasc. Res., 55:146-152, 1998.
Nicolau and Sene, Biochim. Biophys. Acta, 721:185-190, 1982.
Nicolau et al., Methods Enzymol., 149:157-176, 1987.
Nie and Emory, Science, 275:1102-1106, 1997
O'Neal et al., Cancer Lett., 209:171-176, 2004.
Overman and Thomas, Biochemistry, 38:4018-4027, 1999.
Pasqualini and Ruoslahti, Nature, 380:364-366, 1996.
Pasqualini et al., Cancer Res., 60:722-727, 2000.
Pasqualini et al., In: Phage Display, A Laboratory Manual, Barbas et al. (Eds.), NY, Cold Spring Harbor Laboratory Press, 22:1-24, 2000.
Pasqualini et al., Nat. Biotechnol., 15:542-546, 1997.
PCT Appln. PCT/GB91/01134
PCT Appln. WO 90/02809
PCT Appln. WO 91/10737
PCT Appln. WO 92/01047
PCT Appln. WO 92/18619
PCT Appln. WO 93/11236
PCT Appln. WO 95/15982
PCT Appln. WO 95/20401
Pelletier and Sonenberg, Nature, 334(6180):320-325, 1988.
Persic et al., Gene, 187 9-18, 1997.
Ping et al., Microcirculation, 3(2):225-228, 1996.
Potter et al., Proc. Natl. Acad. Sci. USA, 81:7161-7165, 1984.
Purdy and Fraden, Phys. Rev., E70:161703-1-161703-8, 2004.
Radler et al., Science, 275:810-814, 1997.
Rajotte et al., J Clin Invest 102:430-437, 1998.
Reches and Gazit, Science, 280:625-627, 2003.
Ridgeway, In: Vectors: A survey of molecular cloning vectors and their uses, Stoneham: Butterworth, pp. 467-492, 1988.
Rippe, et al., Mol. Cell Biol., 10:689-695, 1990.
Sambrook et al., In: Molecular cloning, Cold Spring Harbor Laboratory Press, Cold Spring Harbor, N.Y., 2001.
Schwartzberg et al., J. Phys. Chem. B, 108:19191-19197, 2004.
Seeman and Belcher, Proc. Natl. Acad. Sci. USA, 99(2):6451-6455, 2002.
Sharma and McQueen, Biochem. Pharm., 29:2017-2021, 1980.
Shipway et al., Langmuir, 16:8789-8795, 2000.
Shipway et al., Langmuir, 16:8789-8795, 2000.
Smith and Scott, Methods Enzymol., 217:228-257, 1993.
Smith and Scott, Science, 228:1315-1317, 1985.
Snyder et al., Cell, 68:33-51, 1992.
Snyder et al., Cell, 68:33-51, 1992.
Souza and Miller, J. Am. Chem. Soc., 123:6734-6735, 2001.
Tang et al., Biophys. J., 83:566-581, 2002.
Tur-Kaspa et al., Mol. Cell Biol., 6:716-718, 1986.
Ulman, Chem. Rev., 96:1533-1554, 1996.
Weisbecker et al., Langmuir, 12:3763-3772, 1996.
West et al., Science, 284:1677-1679, 1999.
Whaley et al., Nature, 405:665, 2000.
Wu and Wu, J. Biol. Chem., 262:4429-4432, 1987.
Wu et al., Anal. Chem., 70:456A, 1998.
Xiao, et al., J. Virol., 70:8098-8108, 1996.
Yang et al., Proc. Natl. Acad. Sci. USA, 87:9568-9572, 1990.
Yguerabide and Yguerabide, Anal. Biochem., 262:137-156, 1998a.
Yguerabide and Yguerabide, Anal. Biochem., 262:157-176, 1998b.
Zimmermann et al., J. Biol. Chem., 261:1653-1655, 1986.
Zurita et al., Cancer Res., 64:435-439, 2004.

1. A bacteriophage assembly comprising a filamentous bacteriophage having a scaffold, wherein the scaffold is associated with a plurality of conductive nanoparticles.

2. The bacteriophage assembly of claim 1, wherein the filamentous bacteriophage is a fd, f1, or M13 bacteriophage.

3. The bacteriophage assembly of claim 2, wherein the bacteriophage is a fd bacteriophage.

4. The bacteriophage assembly of claim 1, further comprising a targeting moiety operably coupled to the bacteriophage or a conductive nanoparticle.

5. (canceled)

6. The bacteriophage assembly of claim 4, wherein the targeting moiety is peptide.

7. The bacteriophage assembly of claim 6, wherein the peptide is a cyclic peptide that is CX₇C peptide, wherein C is cysteine and X is a random amino acid.

8. (canceled)

9. The bacteriophage assembly of claim 4, wherein the targeting moiety is antibody or antibody fragment.

10. The bacteriophage assembly of claim 6, wherein the peptide is comprised in a pIII protein of the bacteriophage.

11. The bacteriophage assembly of claim 1, wherein the conductive nanoparticle is a metallic conductive nanoparticle comprising Au, Ag, Pt, Ti, Al, Si, Ge, Cu, Cr, W, Fe, or a corresponding oxide.

12. (canceled)

13. The bacteriophage assembly of claim 11, wherein the conductive nanoparticle is a Au cluster.

14. The bacteriophage assembly of claim 1, wherein the conductive nanoparticle is 2 to 500 nm in diameter.

15-16. (canceled)

17. The bacteriophage assembly of claim 14, wherein the conductive nanoparticle is 75 to 150 nm in diameter.

18. The bacteriophage assembly of claim 1, further comprising an organizing agent that promotes organized packing of conductive nanoparticles.

19. The bacteriophage assembly of claim 18, wherein the organizing agent is a peptide, a pyrrole, an imidazole, histidine, cysteine, or tryptophan.

20. The bacteriophage assembly of claim 1, further comprising a therapeutic agent.

21. (canceled)

22. The bacteriophage assembly of claim 1, wherein the assembly is comprised in a pharmaceutically acceptable composition.

23. The bacteriophage assembly of claim 1, wherein the bacteriophage assembly is comprised in or bound to a cell.

24. A method of producing a bacteriophage assembly comprising:
contacting filamentous bacteriophage with a conductive atomic or molecular cluster, wherein a bacteriophage assembly is formed; and
isolating the bacteriophage assembly.

25. The method of claim 24, wherein the filamentous bacteriophage is comprised in a solution that has a bacteriophage concentration of between 10⁴ to 10¹² transduction units (TU) per microliter.

26. The method of claim 24, wherein the conductive atomic or molecular cluster has a diameter in the range of 2 nm to 1,000 nm.

27. The method of claim 26, wherein the conductive atomic or molecular cluster is comprised in a solution that has an absorption of 1.2 to 1.5 absorbance units at a wavelength appropriate for the conductive cluster.

28. The method of claim 24, further comprising providing a series of bacteriophage solutions comprising a dilution series of bacteriophage, wherein each solution of the series is mixed individually with a solution of conductive clusters.

29-30. (canceled)

31. The method of claim 25, wherein the bacteriophage solution contains 10⁵ to 10¹⁰ TU per microliter.

32. (canceled)

33. The method of claim 24, wherein the conductive clusters are 2 to 500 nm in diameter.

34-35. (canceled)

36. The method of claim 33, wherein the conductive clusters are 75 to 150 nm in diameter.

37. The method of claim 24, further comprising contacting the bacteriophage or the conductive atomic or molecular cluster with an organizing agent that increases the ratio of conductive clusters to bacteriophage in the assembly.

38. The method of claim 37, wherein the organizing agent is imidazole.

39-43. (canceled)

44. A detection method comprising:
a) contacting a cell with a bacteriophage assembly of claim 1;
b) exposing the cell/bacteriophage assembly complex to a radiation source; and
c) detecting a signal produced by the cell associated bacteriophage assembly.

45. The method of claim 44, wherein the radiation source is an infrared radiation source.

46. The method of claim 44, wherein the cell is comprised in a tissue, an organ, or an organism.

47. The method of claim 44, wherein the cell is comprised in a tissue sample.

48. (canceled)

49. The method of claim 44, wherein the cell is comprised in a fluid sample and the fluid sample is analyzed by flow assisted cell sorting.

50. (canceled)

51. The method of claim 49, wherein the cell is sorted by the presence or absence of a detectable signal.

52. The method of claim 44, wherein the detected signal is a Raman, enhanced fluorescence, absorption or elastic scattering signal.

53. The method of claim 44, wherein the cell associated bacteriophage assembly is heated and the cell is incapacitated.

54-56. (canceled)

57. The method of claim 24,
wherein the filamentous bacteriophage is contacted with a cell, forming a bacteriophage/cell complex; and
the cell/bacteriophage complex is contacted with a plurality of conductive clusters, wherein a bacteriophage assembly is formed.

58. The method of claim 57, wherein the cell is affixed to a slide.

59. The method of claim 57, wherein the cell is comprised in a tissue, an organ, or an organism.

60. A kit comprising a filamentous bacteriophage and a conductive atomic or molecular clusters having a diameter of 2 nm to 1,000 nm disposed in a suitable containers.

61. The kit of claim 60, further comprising an organizing agent for inducing closer packing of the conductive clusters and increasing the conductive cluster to bacteriophage ratio in a bacteriophage assembly.


SEQUENCE LISTING (685 sequences)

SEQ ID NO: 1 - protein, 9 aa, Artificial Sequence
  Description of Artificial Sequence  Synthetic Peptide
CGSPGWVRC

SEQ ID NO: 2 - protein, 4 aa, Artificial Sequence
  Description of Artificial Sequence  Synthetic Peptide
PGWV

SEQ ID NO: 3 - protein, 7 aa, Artificial Sequence
  Description of Artificial Sequence  Synthetic Peptide
AAAGSKH

SEQ ID NO: 4 - protein, 7 aa, Artificial Sequence
  Description of Artificial Sequence  Synthetic Peptide
ARGAGLA

SEQ ID NO: 5 - protein, 7 aa, Artificial Sequence
  Description of Artificial Sequence  Synthetic Peptide
ARGVNGA

SEQ ID NO: 6 - protein, 7 aa, Artificial Sequence
  Description of Artificial Sequence  Synthetic Peptide
AERLWRS

SEQ ID NO: 7 - protein, 9 aa, Artificial Sequence
  Description of Artificial Sequence  Synthetic Peptide
AGWLYMSWK

SEQ ID NO: 8 - protein, 6 aa, Artificial Sequence
  Description of Artificial Sequence  Synthetic Peptide
ALVLQG

SEQ ID NO: 9 - protein, 6 aa, Artificial Sequence
  Description of Artificial Sequence  Synthetic Peptide
AMSAGV

SEQ ID NO: 10 - protein, 7 aa, Artificial Sequence
  Description of Artificial Sequence  Synthetic Peptide
APNTPVL

SEQ ID NO: 11 - protein, 9 aa, Artificial Sequence
  Description of Artificial Sequence  Synthetic Peptide
CRLSLPELC

SEQ ID NO: 12 - protein, 7 aa, Artificial Sequence
  Description of Artificial Sequence  Synthetic Peptide
CRKLGGC

SEQ ID NO: 13 - protein, 9 aa, Artificial Sequence
  Description of Artificial Sequence  Synthetic Peptide
CRMMALHEC

SEQ ID NO: 14 - protein, 9 aa, Artificial Sequence
  Description of Artificial Sequence  Synthetic Peptide
CRSLPPVRC

SEQ ID NO: 15 - protein, 7 aa, Artificial Sequence
  Description of Artificial Sequence  Synthetic Peptide
CRSPMKC

SEQ ID NO: 16 - protein, 9 aa, Artificial Sequence
  Description of Artificial Sequence  Synthetic Peptide
CRSSGFGTC

SEQ ID NO: 17 - protein, 11 aa, Artificial Sequence
  Description of Artificial Sequence  Synthetic Peptide
CRSYVPSTGGC

SEQ ID NO: 18 - protein, 9 aa, Artificial Sequence
  Description of Artificial Sequence  Synthetic Peptide
CRTAVVGSC

SEQ ID NO: 19 - protein, 12 aa, Artificial Sequence
  Description of Artificial Sequence  Synthetic Peptide
CRTEVWRSNQRC

SEQ ID NO: 20 - protein, 12 aa, Artificial Sequence
  Description of Artificial Sequence  Synthetic Peptide
CRTIDIENNELC

SEQ ID NO: 21 - protein, 6 aa, Artificial Sequence
  Description of Artificial Sequence  Synthetic Peptide
AVSECF

SEQ ID NO: 22 - protein, 9 aa, Artificial Sequence
  Description of Artificial Sequence  Synthetic Peptide
CRWSSMIWC

SEQ ID NO: 23 - protein, 11 aa, Artificial Sequence
  Description of Artificial Sequence  Synthetic Peptide
CRVDAHRVIRC

SEQ ID NO: 24 - protein, 9 aa, Artificial Sequence
  Description of Artificial Sequence  Synthetic Peptide
CRVDFSKGC

SEQ ID NO: 25 - protein, 9 aa, Artificial Sequence
  Description of Artificial Sequence  Synthetic Peptide
CRVIVGPRC

SEQ ID NO: 26 - protein, 9 aa, Artificial Sequence
  Description of Artificial Sequence  Synthetic Peptide
CRVLADRDC

SEQ ID NO: 27 - protein, 9 aa, Artificial Sequence
  Description of Artificial Sequence  Synthetic Peptide
CRVFAGKRC

SEQ ID NO: 28 - protein, 9 aa, Artificial Sequence
  Description of Artificial Sequence  Synthetic Peptide
CRVTRGHGC

SEQ ID NO: 29 - protein, 9 aa, Artificial Sequence
  Description of Artificial Sequence  Synthetic Peptide
CNRMTSYSC

SEQ ID NO: 30 - protein, 8 aa, Artificial Sequence
  Description of Artificial Sequence  Synthetic Peptide
CNNMYAGC

SEQ ID NO: 31 - protein, 12 aa, Artificial Sequence
  Description of Artificial Sequence  Synthetic Peptide
CNNWGELLGFNC

SEQ ID NO: 32 - protein, 6 aa, Artificial Sequence
  Description of Artificial Sequence  Synthetic Peptide
RAGYAA

SEQ ID NO: 33 - protein, 8 aa, Artificial Sequence
  Description of Artificial Sequence  Synthetic Peptide
CNQSMWSC

SEQ ID NO: 34 - protein, 11 aa, Artificial Sequence
  Description of Artificial Sequence  Synthetic Peptide
CNGRCRLASSA

SEQ ID NO: 35 - protein, 11 aa, Artificial Sequence
  Description of Artificial Sequence  Synthetic Peptide
CNGRCRLDYRF

SEQ ID NO: 36 - protein, 11 aa, Artificial Sequence
  Description of Artificial Sequence  Synthetic Peptide
CNGRCSRSGPA

SEQ ID NO: 37 - protein, 11 aa, Artificial Sequence
  Description of Artificial Sequence  Synthetic Peptide
CNGRCTMGVRA

SEQ ID NO: 38 - protein, 11 aa, Artificial Sequence
  Description of Artificial Sequence  Synthetic Peptide
CNGRCTSGRVG

SEQ ID NO: 39 - protein, 11 aa, Artificial Sequence
  Description of Artificial Sequence  Synthetic Peptide
CNGRCTSGPGW

SEQ ID NO: 40 - protein, 8 aa, Artificial Sequence
  Description of Artificial Sequence  Synthetic Peptide
CNGIVQVC

SEQ ID NO: 41 - protein, 8 aa, Artificial Sequence
  Description of Artificial Sequence  Synthetic Peptide
CNGFMGYC

SEQ ID NO: 42 - protein, 8 aa, Artificial Sequence
  Description of Artificial Sequence  Synthetic Peptide
CNIRRQGC

SEQ ID NO: 43 - protein, 9 aa, Artificial Sequence
  Description of Artificial Sequence  Synthetic Peptide
RAILYRLAN

SEQ ID NO: 44 - protein, 8 aa, Artificial Sequence
  Description of Artificial Sequence  Synthetic Peptide
CNITQKSC

SEQ ID NO: 45 - protein, 11 aa, Artificial Sequence
  Description of Artificial Sequence  Synthetic Peptide
CNLAGASRDSC

SEQ ID NO: 46 - protein, 8 aa, Artificial Sequence
  Description of Artificial Sequence  Synthetic Peptide
CNLSSEQC

SEQ ID NO: 47 - protein, 8 aa, Artificial Sequence
  Description of Artificial Sequence  Synthetic Peptide
CNLSVPAC

SEQ ID NO: 48 - protein, 8 aa, Artificial Sequence
  Description of Artificial Sequence  Synthetic Peptide
CNKTDKGC

SEQ ID NO: 49 - protein, 9 aa, Artificial Sequence
  Description of Artificial Sequence  Synthetic Peptide
CNFAGPASC

SEQ ID NO: 50 - protein, 9 aa, Artificial Sequence
  Description of Artificial Sequence  Synthetic Peptide
CNPHKAQSC

SEQ ID NO: 51 - protein, 9 aa, Artificial Sequence
  Description of Artificial Sequence  Synthetic Peptide
CNPLFWWWC

SEQ ID NO: 52 - protein, 8 aa, Artificial Sequence
  Description of Artificial Sequence  Synthetic Peptide
CNSAFAGC

SEQ ID NO: 53 - protein, 9 aa, Artificial Sequence
  Description of Artificial Sequence  Synthetic Peptide
CNSRAGSVC

SEQ ID NO: 54 - protein, 7 aa, Artificial Sequence
  Description of Artificial Sequence  Synthetic Peptide
RAPGGVR

SEQ ID NO: 55 - protein, 8 aa, Artificial Sequence
  Description of Artificial Sequence  Synthetic Peptide
CNWTDKTC

SEQ ID NO: 56 - protein, 9 aa, Artificial Sequence
  Description of Artificial Sequence  Synthetic Peptide
CNVEASVRC

SEQ ID NO: 57 - protein, 8 aa, Artificial Sequence
  Description of Artificial Sequence  Synthetic Peptide
CNVSDKSC

SEQ ID NO: 58 - protein, 7 aa, Artificial Sequence
  Description of Artificial Sequence  Synthetic Peptide
CNVWYGC

SEQ ID NO: 59 - protein, 8 aa, Artificial Sequence
  modified_res (3): xaa = can be any naturally occurring amino acid
CNXSDKSC

SEQ ID NO: 60 - protein, 8 aa, Artificial Sequence
  modified_res (3): xaa = can be any naturally occurring amino acid
CNXTDKSC

SEQ ID NO: 61 - protein, 9 aa, Artificial Sequence
  Description of Artificial Sequence  Synthetic Peptide
CDAGPWTAC

SEQ ID NO: 62 - protein, 9 aa, Artificial Sequence
  Description of Artificial Sequence  Synthetic Peptide
CDAYPLFFC

SEQ ID NO: 63 - protein, 8 aa, Artificial Sequence
  Description of Artificial Sequence  Synthetic Peptide
CDNGNASC

SEQ ID NO: 64 - protein, 9 aa, Artificial Sequence
  Description of Artificial Sequence  Synthetic Peptide
CDCRGDCFC

SEQ ID NO: 65 - protein, 16 aa, Artificial Sequence
  Description of Artificial Sequence  Synthetic Peptide
RDLLHLLAFSKSCSLP

SEQ ID NO: 66 - protein, 9 aa, Artificial Sequence
  Description of Artificial Sequence  Synthetic Peptide
CDQNWLVHC

SEQ ID NO: 67 - protein, 9 aa, Artificial Sequence
  Description of Artificial Sequence  Synthetic Peptide
CDGGLDWVC

SEQ ID NO: 68 - protein, 13 aa, Artificial Sequence
  Description of Artificial Sequence  Synthetic Peptide
CDGVCAPRCGERC

SEQ ID NO: 69 - protein, 9 aa, Artificial Sequence
  Description of Artificial Sequence  Synthetic Peptide
CDGVLMFGC

SEQ ID NO: 70 - protein, 9 aa, Artificial Sequence
  Description of Artificial Sequence  Synthetic Peptide
CDSPRRPVC

SEQ ID NO: 71 - protein, 7 aa, Artificial Sequence
  Description of Artificial Sequence  Synthetic Peptide
CDSWPLC

SEQ ID NO: 72 - protein, 9 aa, Artificial Sequence
  Description of Artificial Sequence  Synthetic Peptide
CDSVVGAWC

SEQ ID NO: 73 - protein, 9 aa, Artificial Sequence
  Description of Artificial Sequence  Synthetic Peptide
CDTNQRVVC

SEQ ID NO: 74 - protein, 9 aa, Artificial Sequence
  Description of Artificial Sequence  Synthetic Peptide
CDWGLWWLC

SEQ ID NO: 75 - protein, 9 aa, Artificial Sequence
  Description of Artificial Sequence  Synthetic Peptide
CDWWTTAWC

SEQ ID NO: 76 - protein, 16 aa, Artificial Sequence
  Description of Artificial Sequence  Synthetic Peptide
RDLLHVLAFSKSCHLP

SEQ ID NO: 77 - protein, 9 aa, Artificial Sequence
  Description of Artificial Sequence  Synthetic Peptide
CCRLQVSHC

SEQ ID NO: 78 - protein, 9 aa, Artificial Sequence
  Description of Artificial Sequence  Synthetic Peptide
CCETITKDC

SEQ ID NO: 79 - protein, 12 aa, Artificial Sequence
  Description of Artificial Sequence  Synthetic Peptide
CCGRKTRGVAIC

SEQ ID NO: 80 - protein, 7 aa, Artificial Sequence
  Description of Artificial Sequence  Synthetic Peptide
CQAWQRC

SEQ ID NO: 81 - protein, 8 aa, Artificial Sequence
  Description of Artificial Sequence  Synthetic Peptide
CQNQYPEC

SEQ ID NO: 82 - protein, 7 aa, Artificial Sequence
  Description of Artificial Sequence  Synthetic Peptide
CQQGRYC

SEQ ID NO: 83 - protein, 9 aa, Artificial Sequence
  Description of Artificial Sequence  Synthetic Peptide
CQGKFSQRC

SEQ ID NO: 84 - protein, 7 aa, Artificial Sequence
  Description of Artificial Sequence  Synthetic Peptide
CQGTWIC

SEQ ID NO: 85 - protein, 9 aa, Artificial Sequence
  Description of Artificial Sequence  Synthetic Peptide
CQHTSGRGC

SEQ ID NO: 86 - protein, 9 aa, Artificial Sequence
  Description of Artificial Sequence  Synthetic Peptide
CQHTSGRGC

SEQ ID NO: 87 - protein, 16 aa, Artificial Sequence
  Description of Artificial Sequence  Synthetic Peptide
RQIKIWFQNRRMKWKK

SEQ ID NO: 88 - protein, 9 aa, Artificial Sequence
  Description of Artificial Sequence  Synthetic Peptide
CQISIWRTC

SEQ ID NO: 89 - protein, 9 aa, Artificial Sequence
  Description of Artificial Sequence  Synthetic Peptide
CQLGRAHGC

SEQ ID NO: 90 - protein, 9 aa, Artificial Sequence
  Description of Artificial Sequence  Synthetic Peptide
CQKLARAGC

SEQ ID NO: 91 - protein, 8 aa, Artificial Sequence
  Description of Artificial Sequence  Synthetic Peptide
CQKLGWRV

SEQ ID NO: 92 - protein, 9 aa, Artificial Sequence
  Description of Artificial Sequence  Synthetic Peptide
CQKFARPGC

SEQ ID NO: 93 - protein, 9 aa, Artificial Sequence
  Description of Artificial Sequence  Synthetic Peptide
CQKVARPGC

SEQ ID NO: 94 - protein, 8 aa, Artificial Sequence
  Description of Artificial Sequence  Synthetic Peptide
CQFENGTC

SEQ ID NO: 95 - protein, 9 aa, Artificial Sequence
  Description of Artificial Sequence  Synthetic Peptide
CQSDCIDLC

SEQ ID NO: 96 - protein, 9 aa, Artificial Sequence
  Description of Artificial Sequence  Synthetic Peptide
CQWWLGPLC

SEQ ID NO: 97 - protein, 7 aa, Artificial Sequence
  Description of Artificial Sequence  Synthetic Peptide
CQVRLSC

SEQ ID NO: 98 - protein, 4 aa, Artificial Sequence
  Description of Artificial Sequence  Synthetic Peptide
RGAG

SEQ ID NO: 99 - protein, 8 aa, Artificial Sequence
  Description of Artificial Sequence  Synthetic Peptide
CEAGRGGC

SEQ ID NO: 100 - protein, 9 aa, Artificial Sequence
  Description of Artificial Sequence  Synthetic Peptide
CEALRLRAC

SEQ ID NO: 101 - protein, 9 aa, Artificial Sequence
  Description of Artificial Sequence  Synthetic Peptide
CERGRGAAC

SEQ ID NO: 102 - protein, 9 aa, Artificial Sequence
  Description of Artificial Sequence  Synthetic Peptide
CERPPFLDC

SEQ ID NO: 103 - protein, 9 aa, Artificial Sequence
  Description of Artificial Sequence  Synthetic Peptide
CERSIGFAC

SEQ ID NO: 104 - protein, 7 aa, Artificial Sequence
  Description of Artificial Sequence  Synthetic Peptide
CENGSRC

SEQ ID NO: 105 - protein, 9 aa, Artificial Sequence
  Description of Artificial Sequence  Synthetic Peptide
CEDSSRANC

SEQ ID NO: 106 - protein, 9 aa, Artificial Sequence
  Description of Artificial Sequence  Synthetic Peptide
CECRGDCYC

SEQ ID NO: 107 - protein, 10 aa, Artificial Sequence
  Description of Artificial Sequence  Synthetic Peptide
CEQRQTQEGC

SEQ ID NO: 108 - protein, 9 aa, Artificial Sequence
  Description of Artificial Sequence  Synthetic Peptide
CEGAGLVAC

SEQ ID NO: 109 - protein, 8 aa, Artificial Sequence
  Description of Artificial Sequence  Synthetic Peptide
RGAGTGWT

SEQ ID NO: 110 - protein, 8 aa, Artificial Sequence
  Description of Artificial Sequence  Synthetic Peptide
CEGESASC

SEQ ID NO: 111 - protein, 12 aa, Artificial Sequence
  Description of Artificial Sequence  Synthetic Peptide
CEGGYENLVLKC

SEQ ID NO: 112 - protein, 7 aa, Artificial Sequence
  Description of Artificial Sequence  Synthetic Peptide
CEGHQSC

SEQ ID NO: 113 - protein, 9 aa, Artificial Sequence
  Description of Artificial Sequence  Synthetic Peptide
CEGVVGIVC

SEQ ID NO: 114 - protein, 10 aa, Artificial Sequence
  Description of Artificial Sequence  Synthetic Peptide
CELREQVGRC

SEQ ID NO: 115 - protein, 9 aa, Artificial Sequence
  Description of Artificial Sequence  Synthetic Peptide
CELRGVVVC

SEQ ID NO: 116 - protein, 10 aa, Artificial Sequence
  Description of Artificial Sequence  Synthetic Peptide
CELRLNSILC

SEQ ID NO: 117 - protein, 9 aa, Artificial Sequence
  Description of Artificial Sequence  Synthetic Peptide
CELGFELGC

SEQ ID NO: 118 - protein, 7 aa, Artificial Sequence
  Description of Artificial Sequence  Synthetic Peptide
CEKRWGC

SEQ ID NO: 119 - protein, 10 aa, Artificial Sequence
  Description of Artificial Sequence  Synthetic Peptide
CEFRLNSILC

SEQ ID NO: 120 - protein, 7 aa, Artificial Sequence
  Description of Artificial Sequence  Synthetic Peptide
RGEVLWS

SEQ ID NO: 121 - protein, 9 aa, Artificial Sequence
  Description of Artificial Sequence  Synthetic Peptide
CEFGLSEVC

SEQ ID NO: 122 - protein, 10 aa, Artificial Sequence
  Description of Artificial Sequence  Synthetic Peptide
CEPYWDGWFC

SEQ ID NO: 123 - protein, 9 aa, Artificial Sequence
  Description of Artificial Sequence  Synthetic Peptide
CESRLVRGC

SEQ ID NO: 124 - protein, 9 aa, Artificial Sequence
  Description of Artificial Sequence  Synthetic Peptide
CESLSHVDC

SEQ ID NO: 125 - protein, 11 aa, Artificial Sequence
  Description of Artificial Sequence  Synthetic Peptide
CETGRIDRSDC

SEQ ID NO: 126 - protein, 9 aa, Artificial Sequence
  Description of Artificial Sequence  Synthetic Peptide
CEWSGIWAC

SEQ ID NO: 127 - protein, 9 aa, Artificial Sequence
  Description of Artificial Sequence  Synthetic Peptide
CEWWPEWLC

SEQ ID NO: 128 - protein, 13 aa, Artificial Sequence
  Description of Artificial Sequence  Synthetic Peptide
CEVGLSAAMAYCC

SEQ ID NO: 129 - protein, 13 aa, Artificial Sequence
  Description of Artificial Sequence  Synthetic Peptide
CEVLCGVDCSNRC

SEQ ID NO: 130 - protein, 8 aa, Artificial Sequence
  Description of Artificial Sequence  Synthetic Peptide
CGAQSPRC

SEQ ID NO: 131 - protein, 6 aa, Artificial Sequence
  Description of Artificial Sequence  Synthetic Peptide
RLTPFR

SEQ ID NO: 132 - protein, 13 aa, Artificial Sequence
  Description of Artificial Sequence  Synthetic Peptide
CGAGCGVPCPGGC

SEQ ID NO: 133 - protein, 9 aa, Artificial Sequence
  Description of Artificial Sequence  Synthetic Peptide
CGAMWGMGC

SEQ ID NO: 134 - protein, 8 aa, Artificial Sequence
  Description of Artificial Sequence  Synthetic Peptide
CGRAKVRC

SEQ ID NO: 135 - protein, 9 aa, Artificial Sequence
  Description of Artificial Sequence  Synthetic Peptide
CGRRDTVDC

SEQ ID NO: 136 - protein, 8 aa, Artificial Sequence
  Description of Artificial Sequence  Synthetic Peptide
CGRDSKQC

SEQ ID NO: 137 - protein, 7 aa, Artificial Sequence
  Description of Artificial Sequence  Synthetic Peptide
CGREGPC

SEQ ID NO: 138 - protein, 9 aa, Artificial Sequence
  Description of Artificial Sequence  Synthetic Peptide
CGRKNEWAC

SEQ ID NO: 139 - protein, 9 aa, Artificial Sequence
  Description of Artificial Sequence  Synthetic Peptide
CGRVNSVAC

SEQ ID NO: 140 - protein, 8 aa, Artificial Sequence
  Description of Artificial Sequence  Synthetic Peptide
CGRVPQMC

SEQ ID NO: 141 - protein, 7 aa, Artificial Sequence
  Description of Artificial Sequence  Synthetic Peptide
CGNHQKC

SEQ ID NO: 142 - protein, 7 aa, Artificial Sequence
  Description of Artificial Sequence  Synthetic Peptide
RLTYKLQ

SEQ ID NO: 143 - protein, 12 aa, Artificial Sequence
  Description of Artificial Sequence  Synthetic Peptide
CGNMGGSLYYVC

SEQ ID NO: 144 - protein, 9 aa, Artificial Sequence
  Description of Artificial Sequence  Synthetic Peptide
CGQEISGLC

SEQ ID NO: 145 - protein, 12 aa, Artificial Sequence
  Description of Artificial Sequence  Synthetic Peptide
CGQGFAPRNLVC

SEQ ID NO: 146 - protein, 9 aa, Artificial Sequence
  Description of Artificial Sequence  Synthetic Peptide
CGQMNREVC

SEQ ID NO: 147 - protein, 12 aa, Artificial Sequence
  Description of Artificial Sequence  Synthetic Peptide
CGEGFGSEWPPC

SEQ ID NO: 148 - protein, 9 aa, Artificial Sequence
  Description of Artificial Sequence  Synthetic Peptide
CGGRAILLC

SEQ ID NO: 149 - protein, 9 aa, Artificial Sequence
  Description of Artificial Sequence  Synthetic Peptide
CGGREFWLC

SEQ ID NO: 150 - protein, 9 aa, Artificial Sequence
  Description of Artificial Sequence  Synthetic Peptide
CGGRPVRGC

SEQ ID NO: 151 - protein, 9 aa, Artificial Sequence
  Description of Artificial Sequence  Synthetic Peptide
CGGEEGRRC

SEQ ID NO: 152 - protein, 9 aa, Artificial Sequence
  Description of Artificial Sequence  Synthetic Peptide
CGGIGPRFC

SEQ ID NO: 153 - protein, 7 aa, Artificial Sequence
  Description of Artificial Sequence  Synthetic Peptide
RMDGPVR

SEQ ID NO: 154 - protein, 9 aa, Artificial Sequence
  Description of Artificial Sequence  Synthetic Peptide
CGGLATTTC

SEQ ID NO: 155 - protein, 9 aa, Artificial Sequence
  Description of Artificial Sequence  Synthetic Peptide
CGGLDVRMC

SEQ ID NO: 156 - protein, 9 aa, Artificial Sequence
  Description of Artificial Sequence  Synthetic Peptide
CGGLVAYGC

SEQ ID NO: 157 - protein, 7 aa, Artificial Sequence
  Description of Artificial Sequence  Synthetic Peptide
CGGSSQC

SEQ ID NO: 158 - protein, 9 aa, Artificial Sequence
  Description of Artificial Sequence  Synthetic Peptide
CGGSYDEVC

SEQ ID NO: 159 - protein, 9 aa, Artificial Sequence
  Description of Artificial Sequence  Synthetic Peptide
CGGWLFPPC

SEQ ID NO: 160 - protein, 9 aa, Artificial Sequence
  Description of Artificial Sequence  Synthetic Peptide
CGGWVDHRC

SEQ ID NO: 161 - protein, 9 aa, Artificial Sequence
  Description of Artificial Sequence  Synthetic Peptide
CGGVHALRC

SEQ ID NO: 162 - protein, 9 aa, Artificial Sequence
  Description of Artificial Sequence  Synthetic Peptide
CGHGVTGRC

SEQ ID NO: 163 - protein, 9 aa, Artificial Sequence
  Description of Artificial Sequence  Synthetic Peptide
CGHLGSVYC

SEQ ID NO: 164 - protein, 7 aa, Artificial Sequence
  Description of Artificial Sequence  Synthetic Peptide
RVGTWGR

SEQ ID NO: 165 - protein, 9 aa, Artificial Sequence
  Description of Artificial Sequence  Synthetic Peptide
CGIRGPNKC

SEQ ID NO: 166 - protein, 13 aa, Artificial Sequence
  Description of Artificial Sequence  Synthetic Peptide
CGLRCPLVCPGGC

SEQ ID NO: 167 - protein, 6 aa, Artificial Sequence
  Description of Artificial Sequence  Synthetic Peptide
CGLSLK

SEQ ID NO: 168 - protein, 9 aa, Artificial Sequence
  Description of Artificial Sequence  Synthetic Peptide
CGLVITATC

SEQ ID NO: 169 - protein, 9 aa, Artificial Sequence
  Description of Artificial Sequence  Synthetic Peptide
CGLVSGPWC

SEQ ID NO: 170 - protein, 13 aa, Artificial Sequence
  Description of Artificial Sequence  Synthetic Peptide
CGFECVRQCPERC

SEQ ID NO: 171 - protein, 8 aa, Artificial Sequence
  Description of Artificial Sequence  Synthetic Peptide
CGSDRWLC

SEQ ID NO: 172 - protein, 9 aa, Artificial Sequence
  Description of Artificial Sequence  Synthetic Peptide
CGSPGWVRC

SEQ ID NO: 173 - protein, 8 aa, Artificial Sequence
  Description of Artificial Sequence  Synthetic Peptide
CGTAEGVC

SEQ ID NO: 174 - protein, 13 aa, Artificial Sequence
  Description of Artificial Sequence  Synthetic Peptide
CGTGCAVECEVVC

SEQ ID NO: 175 - protein, 5 aa, Artificial Sequence
  modified_res (4): xaa = can be any naturally occurring amino acid
RVTXG

SEQ ID NO: 176 - protein, 12 aa, Artificial Sequence
  Description of Artificial Sequence  Synthetic Peptide
CGTGCAVECVVC

SEQ ID NO: 177 - protein, 9 aa, Artificial Sequence
  Description of Artificial Sequence  Synthetic Peptide
CGWFSWFGC

SEQ ID NO: 178 - protein, 9 aa, Artificial Sequence
  Description of Artificial Sequence  Synthetic Peptide
CGWFSWWGC

SEQ ID NO: 179 - protein, 9 aa, Artificial Sequence
  Description of Artificial Sequence  Synthetic Peptide
CGWWGLWPC

SEQ ID NO: 180 - protein, 8 aa, Artificial Sequence
  Description of Artificial Sequence  Synthetic Peptide
CGVQYVNC

SEQ ID NO: 181 - protein, 9 aa, Artificial Sequence
  Description of Artificial Sequence  Synthetic Peptide
CGVLKPYLC

SEQ ID NO: 182 - protein, 13 aa, Artificial Sequence
  Description of Artificial Sequence  Synthetic Peptide
CGVPCRPACRGLC

SEQ ID NO: 183 - protein, 7 aa, Artificial Sequence
  Description of Artificial Sequence  Synthetic Peptide
CGVVKFA

SEQ ID NO: 184 - protein, 9 aa, Artificial Sequence
  Description of Artificial Sequence  Synthetic Peptide
CGVVSRVGC

SEQ ID NO: 185 - protein, 12 aa, Artificial Sequence
  Description of Artificial Sequence  Synthetic Peptide
CHRGINRSTTDC

SEQ ID NO: 186 - protein, 7 aa, Artificial Sequence
  Description of Artificial Sequence  Synthetic Peptide
RVVGSRS

SEQ ID NO: 187 - protein, 9 aa, Artificial Sequence
  Description of Artificial Sequence  Synthetic Peptide
CHDERTGRC

SEQ ID NO: 188 - protein, 8 aa, Artificial Sequence
  Description of Artificial Sequence  Synthetic Peptide
CHQKPWEC

SEQ ID NO: 189 - protein, 9 aa, Artificial Sequence
  Description of Artificial Sequence  Synthetic Peptide
CHLEPDWVC

SEQ ID NO: 190 - protein, 10 aa, Artificial Sequence
  Description of Artificial Sequence  Synthetic Peptide
CHSLLEKGWC

SEQ ID NO: 191 - protein, 9 aa, Artificial Sequence
  Description of Artificial Sequence  Synthetic Peptide
CHTWGGRNC

SEQ ID NO: 192 - protein, 12 aa, Artificial Sequence
  Description of Artificial Sequence  Synthetic Peptide
CHVRDEHHEQGC

SEQ ID NO: 193 - protein, 9 aa, Artificial Sequence
  Description of Artificial Sequence  Synthetic Peptide
CHVLWSTRC

SEQ ID NO: 194 - protein, 8 aa, Artificial Sequence
  Description of Artificial Sequence  Synthetic Peptide
CIRREKRC

SEQ ID NO: 195 - protein, 9 aa, Artificial Sequence
  Description of Artificial Sequence  Synthetic Peptide
CIREGMWGC

SEQ ID NO: 196 - protein, 9 aa, Artificial Sequence
  Description of Artificial Sequence  Synthetic Peptide
CIRKGMWGC

SEQ ID NO: 197 - protein, 6 aa, Artificial Sequence
  Description of Artificial Sequence  Synthetic Peptide
DRVTLG

SEQ ID NO: 198 - protein, 8 aa, Artificial Sequence
  Description of Artificial Sequence  Synthetic Peptide
CIRPNDHC

SEQ ID NO: 199 - protein, 9 aa, Artificial Sequence
  Description of Artificial Sequence  Synthetic Peptide
CIRVEAGSC

SEQ ID NO: 200 - protein, 8 aa, Artificial Sequence
  Description of Artificial Sequence  Synthetic Peptide
CIDMTHQC

SEQ ID NO: 201 - protein, 8 aa, Artificial Sequence
  Description of Artificial Sequence  Synthetic Peptide
CIDTSELC

SEQ ID NO: 202 - protein, 11 aa, Artificial Sequence
  Description of Artificial Sequence  Synthetic Peptide
CIQALDASGVC

SEQ ID NO: 203 - protein, 9 aa, Artificial Sequence
  Description of Artificial Sequence  Synthetic Peptide
CIEGRRGLC

SEQ ID NO: 204 - protein, 9 aa, Artificial Sequence
  Description of Artificial Sequence  Synthetic Peptide
CIEWGSRDC

SEQ ID NO: 205 - protein, 9 aa, Artificial Sequence
  Description of Artificial Sequence  Synthetic Peptide
CIGGVHYAC

SEQ ID NO: 206 - protein, 9 aa, Artificial Sequence
  Description of Artificial Sequence  Synthetic Peptide
CIGLSGIEC

SEQ ID NO: 207 - protein, 9 aa, Artificial Sequence
  Description of Artificial Sequence  Synthetic Peptide
CIGKGTSLC

SEQ ID NO: 208 - protein, 9 aa, Artificial Sequence
  Description of Artificial Sequence  Synthetic Peptide
DEEGYYMMR

SEQ ID NO: 209 - protein, 8 aa, Artificial Sequence
  Description of Artificial Sequence  Synthetic Peptide
CIIGSYVC

SEQ ID NO: 210 - protein, 8 aa, Artificial Sequence
  Description of Artificial Sequence  Synthetic Peptide
CIPGTSLC

SEQ ID NO: 211 - protein, 8 aa, Artificial Sequence
  Description of Artificial Sequence  Synthetic Peptide
CISSPSNC

SEQ ID NO: 212 - protein, 9 aa, Artificial Sequence
  Description of Artificial Sequence  Synthetic Peptide
CIVGAPRLC

SEQ ID NO: 213 - protein, 9 aa, Artificial Sequence
  Description of Artificial Sequence  Synthetic Peptide
CIVMLGWRC

SEQ ID NO: 214 - protein, 9 aa, Artificial Sequence
  Description of Artificial Sequence  Synthetic Peptide
CLAAVYQSC

SEQ ID NO: 215 - protein, 8 aa, Artificial Sequence
  Description of Artificial Sequence  Synthetic Peptide
CLANLQTC

SEQ ID NO: 216 - protein, 9 aa, Artificial Sequence
  Description of Artificial Sequence  Synthetic Peptide
CLAHRVGGC

SEQ ID NO: 217 - protein, 9 aa, Artificial Sequence
  Description of Artificial Sequence  Synthetic Peptide
CLASGMDAC

SEQ ID NO: 218 - protein, 12 aa, Artificial Sequence
  Description of Artificial Sequence  Synthetic Peptide
CLASMLNMSTLC

SEQ ID NO: 219 - protein, 4 aa, Artificial Sequence
  Description of Artificial Sequence  Synthetic Peptide
DGSG

SEQ ID NO: 220 - protein, 9 aa, Artificial Sequence
  Description of Artificial Sequence  Synthetic Peptide
CLAWEVYLC

SEQ ID NO: 221 - protein, 9 aa, Artificial Sequence
  Description of Artificial Sequence  Synthetic Peptide
CLAWVGGRC

SEQ ID NO: 222 - protein, 9 aa, Artificial Sequence
  Description of Artificial Sequence  Synthetic Peptide
CLRQSYSNC

SEQ ID NO: 223 - protein, 10 aa, Artificial Sequence
  Description of Artificial Sequence  Synthetic Peptide
CLRQSYSYNC

SEQ ID NO: 224 - protein, 8 aa, Artificial Sequence
  Description of Artificial Sequence  Synthetic Peptide
CLRGGNLR

SEQ ID NO: 225 - protein, 9 aa, Artificial Sequence
  Description of Artificial Sequence  Synthetic Peptide
CLRLSMSAC

SEQ ID NO: 226 - protein, 9 aa, Artificial Sequence
  Description of Artificial Sequence  Synthetic Peptide
CLRSGGLTC

SEQ ID NO: 227 - protein, 9 aa, Artificial Sequence
  Description of Artificial Sequence  Synthetic Peptide
CLDRGGAHC

SEQ ID NO: 228 - protein, 9 aa, Artificial Sequence
  Description of Artificial Sequence  Synthetic Peptide
CLEWLQQVC

SEQ ID NO: 229 - protein, 9 aa, Artificial Sequence
  Description of Artificial Sequence  Synthetic Peptide
CLGRLTVLC

SEQ ID NO: 230 - protein, 7 aa, Artificial Sequence
  Description of Artificial Sequence  Synthetic Peptide
DGSTNLS

SEQ ID NO: 231 - protein, 9 aa, Artificial Sequence
  Description of Artificial Sequence  Synthetic Peptide
CLGDIVERC

SEQ ID NO: 232 - protein, 13 aa, Artificial Sequence
  Description of Artificial Sequence  Synthetic Peptide
CLGQCASICVNDC

SEQ ID NO: 233 - protein, 9 aa, Artificial Sequence
  Description of Artificial Sequence  Synthetic Peptide
CLGLLMAGC

SEQ ID NO: 234 - protein, 9 aa, Artificial Sequence
  Description of Artificial Sequence  Synthetic Peptide
CLGKGLSSC

SEQ ID NO: 235 - protein, 8 aa, Artificial Sequence
  Description of Artificial Sequence  Synthetic Peptide
CLGKGSVC

SEQ ID NO: 236 - protein, 12 aa, Artificial Sequence
  Description of Artificial Sequence  Synthetic Peptide
CLGKWKSSRGTC

SEQ ID NO: 237 - protein, 9 aa, Artificial Sequence
  Description of Artificial Sequence  Synthetic Peptide
CLGSGSGSC

SEQ ID NO: 238 - protein, 9 aa, Artificial Sequence
  Description of Artificial Sequence  Synthetic Peptide
CLGWRAAAC

SEQ ID NO: 239 - protein, 12 aa, Artificial Sequence
  Description of Artificial Sequence  Synthetic Peptide
CLHWEATFNPQC

SEQ ID NO: 240 - protein, 9 aa, Artificial Sequence
  Description of Artificial Sequence  Synthetic Peptide
CLLSGLMGC

SEQ ID NO: 241 - protein, 7 aa, Artificial Sequence
  Description of Artificial Sequence  Synthetic Peptide
DGYPAIA

SEQ ID NO: 242 - protein, 9 aa, Artificial Sequence
  Description of Artificial Sequence  Synthetic Peptide
CLLVGQVRC

SEQ ID NO: 243 - protein, 9 aa, Artificial Sequence
  Description of Artificial Sequence  Synthetic Peptide
CLKTEFTAC

SEQ ID NO: 244 - protein, 9 aa, Artificial Sequence
  Description of Artificial Sequence  Synthetic Peptide
CLKYEQRPC

SEQ ID NO: 245 - protein, 8 aa, Artificial Sequence
  Description of Artificial Sequence  Synthetic Peptide
CLPISSSC

SEQ ID NO: 246 - protein, 12 aa, Artificial Sequence
  Description of Artificial Sequence  Synthetic Peptide
CLSRLVTGDVIC

SEQ ID NO: 247 - protein, 9 aa, Artificial Sequence
  Description of Artificial Sequence  Synthetic Peptide
CLSKLHYVC

SEQ ID NO: 248 - protein, 8 aa, Artificial Sequence
  Description of Artificial Sequence  Synthetic Peptide
CLSPIGEC

SEQ ID NO: 249 - protein, 9 aa, Artificial Sequence
  Description of Artificial Sequence  Synthetic Peptide
CLSSVRGWC

SEQ ID NO: 250 - protein, 9 aa, Artificial Sequence
  Description of Artificial Sequence  Synthetic Peptide
CLTLKSNVC

SEQ ID NO: 251 - protein, 8 aa, Artificial Sequence
  Description of Artificial Sequence  Synthetic Peptide
CLTSENAC

SEQ ID NO: 252 - protein, 4 aa, Artificial Sequence
  Description of Artificial Sequence  Synthetic Peptide
DLLR

SEQ ID NO: 253 - protein, 9 aa, Artificial Sequence
  Description of Artificial Sequence  Synthetic Peptide
CLWPWAGEC

SEQ ID NO: 254 - protein, 9 aa, Artificial Sequence
  Description of Artificial Sequence  Synthetic Peptide
CLWTQSSGC

SEQ ID NO: 255 - protein, 9 aa, Artificial Sequence
  Description of Artificial Sequence  Synthetic Peptide
CLYDVSGGC

SEQ ID NO: 256 - protein, 8 aa, Artificial Sequence
  Description of Artificial Sequence  Synthetic Peptide
CLVGAVQC

SEQ ID NO: 257 - protein, 9 aa, Artificial Sequence
  Description of Artificial Sequence  Synthetic Peptide
CLVLGEKPC

SEQ ID NO: 258 - protein, 9 aa, Artificial Sequence
  Description of Artificial Sequence  Synthetic Peptide
CLVLGGYGC

SEQ ID NO: 259 - protein, 9 aa, Artificial Sequence
  Description of Artificial Sequence  Synthetic Peptide
CLVLSASAC

SEQ ID NO: 260 - protein, 9 aa, Artificial Sequence
  Description of Artificial Sequence  Synthetic Peptide
CLVSYMNGC

SEQ ID NO: 261 - protein, 8 aa, Artificial Sequence
  Description of Artificial Sequence  Synthetic Peptide
CLVYNPAC

SEQ ID NO: 262 - protein, 9 aa, Artificial Sequence
  Description of Artificial Sequence  Synthetic Peptide
CKAFQRHHC

SEQ ID NO: 263 - protein, 7 aa, Artificial Sequence
  Description of Artificial Sequence  Synthetic Peptide
DLLRARW

SEQ ID NO: 264 - protein, 9 aa, Artificial Sequence
  Description of Artificial Sequence  Synthetic Peptide
CKRSATILC

SEQ ID NO: 265 - protein, 8 aa, Artificial Sequence
  Description of Artificial Sequence  Synthetic Peptide
CKNKHTTC

SEQ ID NO: 266 - protein, 8 aa, Artificial Sequence
  Description of Artificial Sequence  Synthetic Peptide
CKDRFERC

SEQ ID NO: 267 - protein, 7 aa, Artificial Sequence
  Description of Artificial Sequence  Synthetic Peptide
CKDRWGC

SEQ ID NO: 268 - protein, 7 aa, Artificial Sequence
  Description of Artificial Sequence  Synthetic Peptide
CKQRGVC

SEQ ID NO: 269 - protein, 12 aa, Artificial Sequence
  Description of Artificial Sequence  Synthetic Peptide
CKERMYREQRRC

SEQ ID NO: 270 - protein, 10 aa, Artificial Sequence
  Description of Artificial Sequence  Synthetic Peptide
CKEDGWLMTC

SEQ ID NO: 271 - protein, 9 aa, Artificial Sequence
  Description of Artificial Sequence  Synthetic Peptide
CKGGRAKDC

SEQ ID NO: 272 - protein, 9 aa, Artificial Sequence
  Description of Artificial Sequence  Synthetic Peptide
CKGSLDEIC

SEQ ID NO: 273 - protein, 8 aa, Artificial Sequence
  Description of Artificial Sequence  Synthetic Peptide
CKLSGTRC

SEQ ID NO: 274 - protein, 7 aa, Artificial Sequence
  Description of Artificial Sequence  Synthetic Peptide
DLLRTEW

SEQ ID NO: 275 - protein, 9 aa, Artificial Sequence
  Description of Artificial Sequence  Synthetic Peptide
CKLWQEDGY

SEQ ID NO: 276 - protein, 12 aa, Artificial Sequence
  Description of Artificial Sequence  Synthetic Peptide
CKPDYMDSNKMC

SEQ ID NO: 277 - protein, 9 aa, Artificial Sequence
  Description of Artificial Sequence  Synthetic Peptide
CKSGQIAVC

SEQ ID NO: 278 - protein, 8 aa, Artificial Sequence
  Description of Artificial Sequence  Synthetic Peptide
CMARQARC

SEQ ID NO: 279 - protein, 7 aa, Artificial Sequence
  Description of Artificial Sequence  Synthetic Peptide
CMATLRC

SEQ ID NO: 280 - protein, 8 aa, Artificial Sequence
  Description of Artificial Sequence  Synthetic Peptide
CMRGKGLC

SEQ ID NO: 281 - protein, 9 aa, Artificial Sequence
  Description of Artificial Sequence  Synthetic Peptide
CMRVTKTHC

SEQ ID NO: 282 - protein, 9 aa, Artificial Sequence
  Description of Artificial Sequence  Synthetic Peptide
CMQGAATSC

SEQ ID NO: 283 - protein, 9 aa, Artificial Sequence
  Description of Artificial Sequence  Synthetic Peptide
CMQGARTSC

SEQ ID NO: 284 - protein, 9 aa, Artificial Sequence
  Description of Artificial Sequence  Synthetic Peptide
CMESSSGLC

SEQ ID NO: 285 - protein, 16 aa, Artificial Sequence
  Description of Artificial Sequence  Synthetic Peptide
DLLHLLAFSKSCSLLP

SEQ ID NO: 286 - protein, 8 aa, Artificial Sequence
  Description of Artificial Sequence  Synthetic Peptide
CMGQGQAC

SEQ ID NO: 287 - protein, 8 aa, Artificial Sequence
  Description of Artificial Sequence  Synthetic Peptide
CMGTKSSC

SEQ ID NO: 288 - protein, 9 aa, Artificial Sequence
  Description of Artificial Sequence  Synthetic Peptide
CMIAGLGRC

SEQ ID NO: 289 - protein, 9 aa, Artificial Sequence
  Description of Artificial Sequence  Synthetic Peptide
CMLVKRKNC

SEQ ID NO: 290 - protein, 9 aa, Artificial Sequence
  Description of Artificial Sequence  Synthetic Peptide
CMMHGLAAC

SEQ ID NO: 291 - protein, 9 aa, Artificial Sequence
  Description of Artificial Sequence  Synthetic Peptide
CMSGNTERC

SEQ ID NO: 292 - protein, 9 aa, Artificial Sequence
  Description of Artificial Sequence  Synthetic Peptide
CMSSPGVAC

SEQ ID NO: 293 - protein, 9 aa, Artificial Sequence
  Description of Artificial Sequence  Synthetic Peptide
CMWGLSYLC

SEQ ID NO: 294 - protein, 9 aa, Artificial Sequence
  Description of Artificial Sequence  Synthetic Peptide
CMYRTSLAC

SEQ ID NO: 295 - protein, 8 aa, Artificial Sequence
  Description of Artificial Sequence  Synthetic Peptide
CMVGYIVC

SEQ ID NO: 296 - protein, 8 aa, Artificial Sequence
  Description of Artificial Sequence  Synthetic Peptide
DWFSYYEL

SEQ ID NO: 297 - protein, 9 aa, Artificial Sequence
  Description of Artificial Sequence  Synthetic Peptide
CMVSGVLLC

SEQ ID NO: 298 - protein, 7 aa, Artificial Sequence
  Description of Artificial Sequence  Synthetic Peptide
CMVYFGC

SEQ ID NO: 299 - protein, 9 aa, Artificial Sequence
  Description of Artificial Sequence  Synthetic Peptide
CFNRTWIGC

SEQ ID NO: 300 - protein, 6 aa, Artificial Sequence
  Description of Artificial Sequence  Synthetic Peptide
CFQNRC

SEQ ID NO: 301 - protein, 10 aa, Artificial Sequence
  Description of Artificial Sequence  Synthetic Peptide
CFELGFELGC

SEQ ID NO: 302 - protein, 8 aa, Artificial Sequence
  Description of Artificial Sequence  Synthetic Peptide
CFETFAGC

SEQ ID NO: 303 - protein, 9 aa, Artificial Sequence
  Description of Artificial Sequence  Synthetic Peptide
CFGGVGSWC

SEQ ID NO: 304 - protein, 9 aa, Artificial Sequence
  Description of Artificial Sequence  Synthetic Peptide
CFHMPPNTC

SEQ ID NO: 305 - protein, 9 aa, Artificial Sequence
  Description of Artificial Sequence  Synthetic Peptide
CFFLRDWFC

SEQ ID NO: 306 - protein, 9 aa, Artificial Sequence
  Description of Artificial Sequence  Synthetic Peptide
CFPRVWSRC

SEQ ID NO: 307 - protein, 8 aa, Artificial Sequence
  Description of Artificial Sequence  Synthetic Peptide
CAAAPIRC

SEQ ID NO: 308 - protein, 8 aa, Artificial Sequence
  Description of Artificial Sequence  Synthetic Peptide
CFPKRVTC

SEQ ID NO: 309 - protein, 9 aa, Artificial Sequence
  Description of Artificial Sequence  Synthetic Peptide
CFSSFFRCC

SEQ ID NO: 310 - protein, 9 aa, Artificial Sequence
  Description of Artificial Sequence  Synthetic Peptide
CFTAVEQGC

SEQ ID NO: 311 - protein, 9 aa, Artificial Sequence
  Description of Artificial Sequence  Synthetic Peptide
CFWPFESWC

SEQ ID NO: 312 - protein, 12 aa, Artificial Sequence
  Description of Artificial Sequence  Synthetic Peptide
CFWTRENRGWTC

SEQ ID NO: 313 - protein, 9 aa, Artificial Sequence
  Description of Artificial Sequence  Synthetic Peptide
CFVFEAMGC

SEQ ID NO: 314 - protein, 9 aa, Artificial Sequence
  Description of Artificial Sequence  Synthetic Peptide
CPRECESIC

SEQ ID NO: 315 - protein, 8 aa, Artificial Sequence
  Description of Artificial Sequence  Synthetic Peptide
CPRSAKNC

SEQ ID NO: 316 - protein, 9 aa, Artificial Sequence
  Description of Artificial Sequence  Synthetic Peptide
CPRYLFWLC

SEQ ID NO: 317 - protein, 7 aa, Artificial Sequence
  Description of Artificial Sequence  Synthetic Peptide
CPNDSLC

SEQ ID NO: 318 - protein, 9 aa, Artificial Sequence
  Description of Artificial Sequence  Synthetic Peptide
CAAGEGWWC

SEQ ID NO: 319 - protein, 8 aa, Artificial Sequence
  Description of Artificial Sequence  Synthetic Peptide
CPQPRPLC

SEQ ID NO: 320 - protein, 8 aa, Artificial Sequence
  Description of Artificial Sequence  Synthetic Peptide
CPERGTRC

SEQ ID NO: 321 - protein, 9 aa, Artificial Sequence
  Description of Artificial Sequence  Synthetic Peptide
CPEVMGSSC

SEQ ID NO: 322 - protein, 9 aa, Artificial Sequence
  Description of Artificial Sequence  Synthetic Peptide
CPGRLWRAC

SEQ ID NO: 323 - protein, 9 aa, Artificial Sequence
  Description of Artificial Sequence  Synthetic Peptide
CPGPLNPPC

SEQ ID NO: 324 - protein, 9 aa, Artificial Sequence
  Description of Artificial Sequence  Synthetic Peptide
CPGSVRVQC

SEQ ID NO: 325 - protein, 9 aa, Artificial Sequence
  Description of Artificial Sequence  Synthetic Peptide
CPKDSSKMC

SEQ ID NO: 326 - protein, 13 aa, Artificial Sequence
  Description of Artificial Sequence  Synthetic Peptide
CPKVCPRECESNC

SEQ ID NO: 327 - protein, 12 aa, Artificial Sequence
  Description of Artificial Sequence  Synthetic Peptide
CPMQATRNLWHC

SEQ ID NO: 328 - protein, 9 aa, Artificial Sequence
  Description of Artificial Sequence  Synthetic Peptide
CPPLGGSRC

SEQ ID NO: 329 - protein, 9 aa, Artificial Sequence
  Description of Artificial Sequence  Synthetic Peptide
CAAGKDLLC

SEQ ID NO: 330 - protein, 7 aa, Artificial Sequence
  Description of Artificial Sequence  Synthetic Peptide
CPTMTEC

SEQ ID NO: 331 - protein, 9 aa, Artificial Sequence
  Description of Artificial Sequence  Synthetic Peptide
CPWWMMERC

SEQ ID NO: 332 - protein, 9 aa, Artificial Sequence
  Description of Artificial Sequence  Synthetic Peptide
CPWWWFGEC

SEQ ID NO: 333 - protein, 9 aa, Artificial Sequence
  Description of Artificial Sequence  Synthetic Peptide
CPWYWLGWC

SEQ ID NO: 334 - protein, 9 aa, Artificial Sequence
  Description of Artificial Sequence  Synthetic Peptide
CPWYWFGTC

SEQ ID NO: 335 - protein, 9 aa, Artificial Sequence
  Description of Artificial Sequence  Synthetic Peptide
CPYDALASC

SEQ ID NO: 336 - protein, 13 aa, Artificial Sequence
  Description of Artificial Sequence  Synthetic Peptide
CPVGCGGGCRPAC

SEQ ID NO: 337 - protein, 11 aa, Artificial Sequence
  Description of Artificial Sequence  Synthetic Peptide
CSAGATVWQGC

SEQ ID NO: 338 - protein, 9 aa, Artificial Sequence
  Description of Artificial Sequence  Synthetic Peptide
CSAFSVAYC

SEQ ID NO: 339 - protein, 8 aa, Artificial Sequence
  Description of Artificial Sequence  Synthetic Peptide
CSASDLSC

SEQ ID NO: 340 - protein, 9 aa, Artificial Sequence
  Description of Artificial Sequence  Synthetic Peptide
CAAGKGLLC

SEQ ID NO: 341 - protein, 9 aa, Artificial Sequence
  Description of Artificial Sequence  Synthetic Peptide
CSRRPEVVC

SEQ ID NO: 342 - protein, 9 aa, Artificial Sequence
  Description of Artificial Sequence  Synthetic Peptide
CSRRVIGAC

SEQ ID NO: 343 - protein, 9 aa, Artificial Sequence
  Description of Artificial Sequence  Synthetic Peptide
CSRNSVREC

SEQ ID NO: 344 - protein, 9 aa, Artificial Sequence
  Description of Artificial Sequence  Synthetic Peptide
CSRGPAWGC

SEQ ID NO: 345 - protein, 8 aa, Artificial Sequence
  Description of Artificial Sequence  Synthetic Peptide
CSRSSFLC

SEQ ID NO: 346 - protein, 9 aa, Artificial Sequence
  Description of Artificial Sequence  Synthetic Peptide
CSRSVSRLC

SEQ ID NO: 347 - protein, 9 aa, Artificial Sequence
  Description of Artificial Sequence  Synthetic Peptide
CSRWGLGGC

SEQ ID NO: 348 - protein, 9 aa, Artificial Sequence
  Description of Artificial Sequence  Synthetic Peptide
CSNAWVHAC

SEQ ID NO: 349 - protein, 8 aa, Artificial Sequence
  Description of Artificial Sequence  Synthetic Peptide
CSQLKLGC

SEQ ID NO: 350 - protein, 9 aa, Artificial Sequence
  Description of Artificial Sequence  Synthetic Peptide
CSERIARVC

SEQ ID NO: 351 - protein, 5 aa, Artificial Sequence
  Description of Artificial Sequence  Synthetic Peptide
CARAC

SEQ ID NO: 352 - protein, 9 aa, Artificial Sequence
  Description of Artificial Sequence  Synthetic Peptide
CSELGGAGC

SEQ ID NO: 353 - protein, 9 aa, Artificial Sequence
  Description of Artificial Sequence  Synthetic Peptide
CSESQLAWC

SEQ ID NO: 354 - protein, 7 aa, Artificial Sequence
  Description of Artificial Sequence  Synthetic Peptide
CSGRSGC

SEQ ID NO: 355 - protein, 9 aa, Artificial Sequence
  Description of Artificial Sequence  Synthetic Peptide
CSGGSGARC

SEQ ID NO: 356 - protein, 13 aa, Artificial Sequence
  Description of Artificial Sequence  Synthetic Peptide
CSGLCTVQCLEGC

SEQ ID NO: 357 - protein, 5 aa, Artificial Sequence
  Description of Artificial Sequence  Synthetic Peptide
CSGPC

SEQ ID NO: 358 - protein, 9 aa, Artificial Sequence
  Description of Artificial Sequence  Synthetic Peptide
CSGVSTVRC

SEQ ID NO: 359 - protein, 9 aa, Artificial Sequence
  Description of Artificial Sequence  Synthetic Peptide
CSHAVMPWC

SEQ ID NO: 360 - protein, 8 aa, Artificial Sequence
  Description of Artificial Sequence  Synthetic Peptide
CSIRRESC

SEQ ID NO: 361 - protein, 9 aa, Artificial Sequence
  Description of Artificial Sequence  Synthetic Peptide
CSLAVPLAC

SEQ ID NO: 362 - protein, 9 aa, Artificial Sequence
  Description of Artificial Sequence  Synthetic Peptide
CARDRIIAC

SEQ ID NO: 363 - protein, 9 aa, Artificial Sequence
  Description of Artificial Sequence  Synthetic Peptide
CSLQGIVGC

SEQ ID NO: 364 - protein, 9 aa, Artificial Sequence
  Description of Artificial Sequence  Synthetic Peptide
CSLGSFMGC

SEQ ID NO: 365 - protein, 9 aa, Artificial Sequence
  Description of Artificial Sequence  Synthetic Peptide
CSLLPASSC

SEQ ID NO: 366 - protein, 8 aa, Artificial Sequence
  Description of Artificial Sequence  Synthetic Peptide
CSKKFVTC

SEQ ID NO: 367 - protein, 9 aa, Artificial Sequence
  Description of Artificial Sequence  Synthetic Peptide
CSKVGPWWC

SEQ ID NO: 368 - protein, 13 aa, Artificial Sequence
  Description of Artificial Sequence  Synthetic Peptide
CSMMCLEGCDDWC

SEQ ID NO: 369 - protein, 9 aa, Artificial Sequence
  Description of Artificial Sequence  Synthetic Peptide
CSMTKLGAC

SEQ ID NO: 370 - protein, 9 aa, Artificial Sequence
  Description of Artificial Sequence  Synthetic Peptide
CSPPLTRWC

SEQ ID NO: 371 - protein, 9 aa, Artificial Sequence
  Description of Artificial Sequence  Synthetic Peptide
CSSELRAAC

SEQ ID NO: 372 - protein, 9 aa, Artificial Sequence
  Description of Artificial Sequence  Synthetic Peptide
CSSLATVVC

SEQ ID NO: 373 - protein, 10 aa, Artificial Sequence
  Description of Artificial Sequence  Synthetic Peptide
CARLEVLLPC

SEQ ID NO: 374 - protein, 9 aa, Artificial Sequence
  Description of Artificial Sequence  Synthetic Peptide
CSSPFHDSC

SEQ ID NO: 375 - protein, 9 aa, Artificial Sequence
  Description of Artificial Sequence  Synthetic Peptide
CSSVVRLGC

SEQ ID NO: 376 - protein, 8 aa, Artificial Sequence
  Description of Artificial Sequence  Synthetic Peptide
CSTFAGSC

SEQ ID NO: 377 - protein, 9 aa, Artificial Sequence
  Description of Artificial Sequence  Synthetic Peptide
CSWWRFGYC

SEQ ID NO: 378 - protein, 9 aa, Artificial Sequence
  Description of Artificial Sequence  Synthetic Peptide
CSWWNWFGC

SEQ ID NO: 379 - protein, 9 aa, Artificial Sequence
  Description of Artificial Sequence  Synthetic Peptide
CSWWTFGFC

SEQ ID NO: 380 - protein, 9 aa, Artificial Sequence
  Description of Artificial Sequence  Synthetic Peptide
CSWYWWLGC

SEQ ID NO: 381 - protein, 9 aa, Artificial Sequence
  Description of Artificial Sequence  Synthetic Peptide
CSYGRASLC

SEQ ID NO: 382 - protein, 8 aa, Artificial Sequence
  Description of Artificial Sequence  Synthetic Peptide
CSYKANSC

SEQ ID NO: 383 - protein, 8 aa, Artificial Sequence
  Description of Artificial Sequence  Synthetic Peptide
CSYTFAGC

SEQ ID NO: 384 - protein, 9 aa, Artificial Sequence
  Description of Artificial Sequence  Synthetic Peptide
CARYLWSWC

SEQ ID NO: 385 - protein, 9 aa, Artificial Sequence
  Description of Artificial Sequence  Synthetic Peptide
CSVIKRGAC

SEQ ID NO: 386 - protein, 9 aa, Artificial Sequence
  Description of Artificial Sequence  Synthetic Peptide
CSVPVSSSC

SEQ ID NO: 387 - protein, 7 aa, Artificial Sequence
  Description of Artificial Sequence  Synthetic Peptide
CSVWFGC

SEQ ID NO: 388 - protein, 7 aa, Artificial Sequence
  Description of Artificial Sequence  Synthetic Peptide
CSVWWGC

SEQ ID NO: 389 - protein, 7 aa, Artificial Sequence
  Description of Artificial Sequence  Synthetic Peptide
CSVWYGC

SEQ ID NO: 390 - protein, 9 aa, Artificial Sequence
  Description of Artificial Sequence  Synthetic Peptide
CTAWFIESC

SEQ ID NO: 391 - protein, 12 aa, Artificial Sequence
  Description of Artificial Sequence  Synthetic Peptide
CTRNITKSRMMC

SEQ ID NO: 392 - protein, 9 aa, Artificial Sequence
  Description of Artificial Sequence  Synthetic Peptide
CTREVHRSC

SEQ ID NO: 393 - protein, 10 aa, Artificial Sequence
  Description of Artificial Sequence  Synthetic Peptide
CTRSFARKEC

SEQ ID NO: 394 - protein, 9 aa, Artificial Sequence
  Description of Artificial Sequence  Synthetic Peptide
CTRVGPKRC

SEQ ID NO: 395 - protein, 8 aa, Artificial Sequence
  Description of Artificial Sequence  Synthetic Peptide
CADMQGTC

SEQ ID NO: 396 - protein, 9 aa, Artificial Sequence
  Description of Artificial Sequence  Synthetic Peptide
CTRVHGLAC

SEQ ID NO: 397 - protein, 7 aa, Artificial Sequence
  Description of Artificial Sequence  Synthetic Peptide
CTNPQRC

SEQ ID NO: 398 - protein, 9 aa, Artificial Sequence
  Description of Artificial Sequence  Synthetic Peptide
CTNPWSPVC

SEQ ID NO: 399 - protein, 9 aa, Artificial Sequence
  Description of Artificial Sequence  Synthetic Peptide
CTQVRFSGC

SEQ ID NO: 400 - protein, 13 aa, Artificial Sequence
  Description of Artificial Sequence  Synthetic Peptide
CTGGCVVDCLSIC

SEQ ID NO: 401 - protein, 9 aa, Artificial Sequence
  Description of Artificial Sequence  Synthetic Peptide
CTHATWLVC

SEQ ID NO: 402 - protein, 7 aa, Artificial Sequence
  Description of Artificial Sequence  Synthetic Peptide
CTLFRNC

SEQ ID NO: 403 - protein, 8 aa, Artificial Sequence
  Description of Artificial Sequence  Synthetic Peptide
CTFAGSSC

SEQ ID NO: 404 - protein, 9 aa, Artificial Sequence
  Description of Artificial Sequence  Synthetic Peptide
CTFSGHRLC

SEQ ID NO: 405 - protein, 9 aa, Artificial Sequence
  Description of Artificial Sequence  Synthetic Peptide
CTPKTSVTC

SEQ ID NO: 406 - protein, 8 aa, Artificial Sequence
  Description of Artificial Sequence  Synthetic Peptide
CADVLRPC

SEQ ID NO: 407 - protein, 9 aa, Artificial Sequence
  Description of Artificial Sequence  Synthetic Peptide
CTPFIAGGC

SEQ ID NO: 408 - protein, 9 aa, Artificial Sequence
  Description of Artificial Sequence  Synthetic Peptide
CTSAASGLC

SEQ ID NO: 409 - protein, 9 aa, Artificial Sequence
  Description of Artificial Sequence  Synthetic Peptide
CTSWWFWSC

SEQ ID NO: 410 - protein, 10 aa, Artificial Sequence
  Description of Artificial Sequence  Synthetic Peptide
CTTHWGFTLC

SEQ ID NO: 411 - protein, 7 aa, Artificial Sequence
  Description of Artificial Sequence  Synthetic Peptide
CTWDKRC

SEQ ID NO: 412 - protein, 9 aa, Artificial Sequence
  Description of Artificial Sequence  Synthetic Peptide
CTWLGGREC

SEQ ID NO: 413 - protein, 9 aa, Artificial Sequence
  Description of Artificial Sequence  Synthetic Peptide
CTWKGGREC

SEQ ID NO: 414 - protein, 9 aa, Artificial Sequence
  Description of Artificial Sequence  Synthetic Peptide
CWALSGGLC

SEQ ID NO: 415 - protein, 8 aa, Artificial Sequence
  Description of Artificial Sequence  Synthetic Peptide
CWRSVEVC

SEQ ID NO: 416 - protein, 8 aa, Artificial Sequence
  Description of Artificial Sequence  Synthetic Peptide
CWNRGSTC

SEQ ID NO: 417 - protein, 9 aa, Artificial Sequence
  Description of Artificial Sequence  Synthetic Peptide
CACVRSRNC

SEQ ID NO: 418 - protein, 9 aa, Artificial Sequence
  Description of Artificial Sequence  Synthetic Peptide
CWNGGARAC

SEQ ID NO: 419 - protein, 10 aa, Artificial Sequence
  Description of Artificial Sequence  Synthetic Peptide
CWDQNYLDDC

SEQ ID NO: 420 - protein, 8 aa, Artificial Sequence
  Description of Artificial Sequence  Synthetic Peptide
CWQGSVSC

SEQ ID NO: 421 - protein, 9 aa, Artificial Sequence
  Description of Artificial Sequence  Synthetic Peptide
CWENRGQFC

SEQ ID NO: 422 - protein, 8 aa, Artificial Sequence
  Description of Artificial Sequence  Synthetic Peptide
CWGGNQAC

SEQ ID NO: 423 - protein, 9 aa, Artificial Sequence
  Description of Artificial Sequence  Synthetic Peptide
CWGGLSGLC

SEQ ID NO: 424 - protein, 7 aa, Artificial Sequence
  Description of Artificial Sequence  Synthetic Peptide
CWILEQC

SEQ ID NO: 425 - protein, 9 aa, Artificial Sequence
  Description of Artificial Sequence  Synthetic Peptide
CWKGFGWWC

SEQ ID NO: 426 - protein, 9 aa, Artificial Sequence
  Description of Artificial Sequence  Synthetic Peptide
CWSAEWTKC

SEQ ID NO: 427 - protein, 9 aa, Artificial Sequence
  Description of Artificial Sequence  Synthetic Peptide
CWSRGQGGC

SEQ ID NO: 428 - protein, 9 aa, Artificial Sequence
  Description of Artificial Sequence  Synthetic Peptide
CAEAGGPDC

SEQ ID NO: 429 - protein, 9 aa, Artificial Sequence
  Description of Artificial Sequence  Synthetic Peptide
CWSLELLKC

SEQ ID NO: 430 - protein, 9 aa, Artificial Sequence
  Description of Artificial Sequence  Synthetic Peptide
CWSLELSKC

SEQ ID NO: 431 - protein, 9 aa, Artificial Sequence
  Description of Artificial Sequence  Synthetic Peptide
CWSLEFTKC

SEQ ID NO: 432 - protein, 9 aa, Artificial Sequence
  Description of Artificial Sequence  Synthetic Peptide
CWSLESLKC

SEQ ID NO: 433 - protein, 9 aa, Artificial Sequence
  Description of Artificial Sequence  Synthetic Peptide
CWSLEVAKC

SEQ ID NO: 434 - protein, 9 aa, Artificial Sequence
  Description of Artificial Sequence  Synthetic Peptide
CWWGWWGTC

SEQ ID NO: 435 - protein, 9 aa, Artificial Sequence
  Description of Artificial Sequence  Synthetic Peptide
CWWPWGGWC

SEQ ID NO: 436 - protein, 9 aa, Artificial Sequence
  Description of Artificial Sequence  Synthetic Peptide
CWWPWGWGC

SEQ ID NO: 437 - protein, 10 aa, Artificial Sequence
  Description of Artificial Sequence  Synthetic Peptide
CWYTEGRMIC

SEQ ID NO: 438 - protein, 9 aa, Artificial Sequence
  Description of Artificial Sequence  Synthetic Peptide
CWVADGYRC

SEQ ID NO: 439 - protein, 13 aa, Artificial Sequence
  Description of Artificial Sequence  Synthetic Peptide
CAGACPVGCGTGC

SEQ ID NO: 440 - protein, 9 aa, Artificial Sequence
  Description of Artificial Sequence  Synthetic Peptide
CYAAGNVFC

SEQ ID NO: 441 - protein, 9 aa, Artificial Sequence
  Description of Artificial Sequence  Synthetic Peptide
CYRSAGAGC

SEQ ID NO: 442 - protein, 8 aa, Artificial Sequence
  Description of Artificial Sequence  Synthetic Peptide
CYQSSSGC

SEQ ID NO: 443 - protein, 9 aa, Artificial Sequence
  Description of Artificial Sequence  Synthetic Peptide
CYGAWRVSC

SEQ ID NO: 444 - protein, 10 aa, Artificial Sequence
  Description of Artificial Sequence  Synthetic Peptide
CYIWPDSGLC

SEQ ID NO: 445 - protein, 9 aa, Artificial Sequence
  Description of Artificial Sequence  Synthetic Peptide
CYKPVHSPC

SEQ ID NO: 446 - protein, 9 aa, Artificial Sequence
  Description of Artificial Sequence  Synthetic Peptide
CYPGYDSYC

SEQ ID NO: 447 - protein, 9 aa, Artificial Sequence
  Description of Artificial Sequence  Synthetic Peptide
CYSDRNMDC

SEQ ID NO: 448 - protein, 7 aa, Artificial Sequence
  Description of Artificial Sequence  Synthetic Peptide
CYSDKKC

SEQ ID NO: 449 - protein, 9 aa, Artificial Sequence
  Description of Artificial Sequence  Synthetic Peptide
CYWLGGALC

SEQ ID NO: 450 - protein, 9 aa, Artificial Sequence
  Description of Artificial Sequence  Synthetic Peptide
CAGRVALRC

SEQ ID NO: 451 - protein, 9 aa, Artificial Sequence
  Description of Artificial Sequence  Synthetic Peptide
CYYPGEKSC

SEQ ID NO: 452 - protein, 9 aa, Artificial Sequence
  Description of Artificial Sequence  Synthetic Peptide
CYVNVQVSC

SEQ ID NO: 453 - protein, 9 aa, Artificial Sequence
  Description of Artificial Sequence  Synthetic Peptide
CVAADRYVC

SEQ ID NO: 454 - protein, 9 aa, Artificial Sequence
  Description of Artificial Sequence  Synthetic Peptide
CVATWCEKC

SEQ ID NO: 455 - protein, 12 aa, Artificial Sequence
  Description of Artificial Sequence  Synthetic Peptide
CVRNVDQNTNTC

SEQ ID NO: 456 - protein, 9 aa, Artificial Sequence
  Description of Artificial Sequence  Synthetic Peptide
CVRDLLTGC

SEQ ID NO: 457 - protein, 9 aa, Artificial Sequence
  Description of Artificial Sequence  Synthetic Peptide
CVRQGEDAC

SEQ ID NO: 458 - protein, 9 aa, Artificial Sequence
  Description of Artificial Sequence  Synthetic Peptide
CVRSSVVAC

SEQ ID NO: 459 - protein, 9 aa, Artificial Sequence
  Description of Artificial Sequence  Synthetic Peptide
CVRTSSQWC

SEQ ID NO: 460 - protein, 8 aa, Artificial Sequence
  Description of Artificial Sequence  Synthetic Peptide
CVNKYIPC

SEQ ID NO: 461 - protein, 8 aa, Artificial Sequence
  Description of Artificial Sequence  Synthetic Peptide
CAGGGAYC

SEQ ID NO: 462 - protein, 11 aa, Artificial Sequence
  Description of Artificial Sequence  Synthetic Peptide
CVERMRVERQC

SEQ ID NO: 463 - protein, 9 aa, Artificial Sequence
  Description of Artificial Sequence  Synthetic Peptide
CVGAGLHIC

SEQ ID NO: 464 - protein, 9 aa, Artificial Sequence
  Description of Artificial Sequence  Synthetic Peptide
CVGATYSRC

SEQ ID NO: 465 - protein, 9 aa, Artificial Sequence
  Description of Artificial Sequence  Synthetic Peptide
CVGNAKLMC

SEQ ID NO: 466 - protein, 9 aa, Artificial Sequence
  Description of Artificial Sequence  Synthetic Peptide
CVGNDVRGC

SEQ ID NO: 467 - protein, 9 aa, Artificial Sequence
  Description of Artificial Sequence  Synthetic Peptide
CVGGARALC

SEQ ID NO: 468 - protein, 9 aa, Artificial Sequence
  Description of Artificial Sequence  Synthetic Peptide
CVGGVRGGC

SEQ ID NO: 469 - protein, 9 aa, Artificial Sequence
  Description of Artificial Sequence  Synthetic Peptide
CVGLRAPLC

SEQ ID NO: 470 - protein, 9 aa, Artificial Sequence
  Description of Artificial Sequence  Synthetic Peptide
CVGVGRSRC

SEQ ID NO: 471 - protein, 9 aa, Artificial Sequence
  Description of Artificial Sequence  Synthetic Peptide
CVLADPTGC

SEQ ID NO: 472 - protein, 9 aa, Artificial Sequence
  Description of Artificial Sequence  Synthetic Peptide
CAGGSLRVC

SEQ ID NO: 473 - protein, 9 aa, Artificial Sequence
  Description of Artificial Sequence  Synthetic Peptide
CVLGEISWC

SEQ ID NO: 474 - protein, 7 aa, Artificial Sequence
  Description of Artificial Sequence  Synthetic Peptide
CVKQMRC

SEQ ID NO: 475 - protein, 9 aa, Artificial Sequence
  Description of Artificial Sequence  Synthetic Peptide
CVKKGGFWC

SEQ ID NO: 476 - protein, 13 aa, Artificial Sequence
  Description of Artificial Sequence  Synthetic Peptide
CVMTCAPRCFEHC

SEQ ID NO: 477 - protein, 8 aa, Artificial Sequence
  Description of Artificial Sequence  Synthetic Peptide
CVFAILAC

SEQ ID NO: 478 - protein, 9 aa, Artificial Sequence
  Description of Artificial Sequence  Synthetic Peptide
CVPRRWDVC

SEQ ID NO: 479 - protein, 9 aa, Artificial Sequence
  Description of Artificial Sequence  Synthetic Peptide
CVPELGHEC

SEQ ID NO: 480 - protein, 9 aa, Artificial Sequence
  Description of Artificial Sequence  Synthetic Peptide
CVPGLGGLC

SEQ ID NO: 481 - protein, 8 aa, Artificial Sequence
  Description of Artificial Sequence  Synthetic Peptide
CVPMRLQC

SEQ ID NO: 482 - protein, 8 aa, Artificial Sequence
  Description of Artificial Sequence  Synthetic Peptide
CVSRPRAC

SEQ ID NO: 483 - protein, 8 aa, Artificial Sequence
  Description of Artificial Sequence  Synthetic Peptide
CAGKSSNC

SEQ ID NO: 484 - protein, 9 aa, Artificial Sequence
  Description of Artificial Sequence  Synthetic Peptide
CVSNPRWKC

SEQ ID NO: 485 - protein, 9 aa, Artificial Sequence
  Description of Artificial Sequence  Synthetic Peptide
CVSQLGRVC

SEQ ID NO: 486 - protein, 9 aa, Artificial Sequence
  Description of Artificial Sequence  Synthetic Peptide
CVSWWFWGC

SEQ ID NO: 487 - protein, 8 aa, Artificial Sequence
  Description of Artificial Sequence  Synthetic Peptide
CVTGNRGC

SEQ ID NO: 488 - protein, 9 aa, Artificial Sequence
  Description of Artificial Sequence  Synthetic Peptide
CVVKLRNRC

SEQ ID NO: 489 - protein, 6 aa, Artificial Sequence
  Description of Artificial Sequence  Synthetic Peptide
QRLVHP

SEQ ID NO: 490 - protein, 6 aa, Artificial Sequence
  Description of Artificial Sequence  Synthetic Peptide
QVLVHP

SEQ ID NO: 491 - protein, 6 aa, Artificial Sequence
  Description of Artificial Sequence  Synthetic Peptide
QVLVTP

SEQ ID NO: 492 - protein, 6 aa, Artificial Sequence
  Description of Artificial Sequence  Synthetic Peptide
QRLVTP

SEQ ID NO: 493 - protein, 6 aa, Artificial Sequence
  Description of Artificial Sequence  Synthetic Peptide
QVLVHP

SEQ ID NO: 494 - protein, 13 aa, Artificial Sequence
  Description of Artificial Sequence  Synthetic Peptide
CAGFCVPGCHSKC

SEQ ID NO: 495 - protein, 6 aa, Artificial Sequence
  Description of Artificial Sequence  Synthetic Peptide
QVLVTP

SEQ ID NO: 496 - protein, 4 aa, Artificial Sequence
  Description of Artificial Sequence  Synthetic Peptide
EGSG

SEQ ID NO: 497 - protein, 7 aa, Artificial Sequence
  Description of Artificial Sequence  Synthetic Peptide
ELGFELG

SEQ ID NO: 498 - protein, 6 aa, Artificial Sequence
  Description of Artificial Sequence  Synthetic Peptide
ELLSGL

SEQ ID NO: 499 - protein, 7 aa, Artificial Sequence
  Description of Artificial Sequence  Synthetic Peptide
EFRLGSK

SEQ ID NO: 500 - protein, 7 aa, Artificial Sequence
  Description of Artificial Sequence  Synthetic Peptide
EFDLVRQ

SEQ ID NO: 501 - protein, 6 aa, Artificial Sequence
  Description of Artificial Sequence  Synthetic Peptide
EVISGL

SEQ ID NO: 502 - protein, 11 aa, Artificial Sequence
  Description of Artificial Sequence  Synthetic Peptide
GACVRLSACGA

SEQ ID NO: 503 - protein, 7 aa, Artificial Sequence
  Description of Artificial Sequence  Synthetic Peptide
GRGRMTS

SEQ ID NO: 504 - protein, 7 aa, Artificial Sequence
  Description of Artificial Sequence  Synthetic Peptide
GRGRWGS

SEQ ID NO: 505 - protein, 8 aa, Artificial Sequence
  Description of Artificial Sequence  Synthetic Peptide
CAGFSSPC

SEQ ID NO: 506 - protein, 6 aa, Artificial Sequence
  Description of Artificial Sequence  Synthetic Peptide
GRGESP

SEQ ID NO: 507 - protein, 7 aa, Artificial Sequence
  Description of Artificial Sequence  Synthetic Peptide
GRLSGSL

SEQ ID NO: 508 - protein, 7 aa, Artificial Sequence
  Description of Artificial Sequence  Synthetic Peptide
GDSPVES

SEQ ID NO: 509 - protein, 7 aa, Artificial Sequence
  Description of Artificial Sequence  Synthetic Peptide
GCDEGGG

SEQ ID NO: 510 - protein, 7 aa, Artificial Sequence
  Description of Artificial Sequence  Synthetic Peptide
GGGSRIS

SEQ ID NO: 511 - protein, 9 aa, Artificial Sequence
  Description of Artificial Sequence  Synthetic Peptide
GGGSYRHVE

SEQ ID NO: 512 - protein, 4 aa, Artificial Sequence
  Description of Artificial Sequence  Synthetic Peptide
GGLS

SEQ ID NO: 513 - protein, 4 aa, Artificial Sequence
  Description of Artificial Sequence  Synthetic Peptide
GGVH

SEQ ID NO: 514 - protein, 5 aa, Artificial Sequence
  Description of Artificial Sequence  Synthetic Peptide
GHAIE

SEQ ID NO: 515 - protein, 7 aa, Artificial Sequence
  Description of Artificial Sequence  Synthetic Peptide
GLAKLIP

SEQ ID NO: 516 - protein, 8 aa, Artificial Sequence
  Description of Artificial Sequence  Synthetic Peptide
CAGYAVDC

SEQ ID NO: 517 - protein, 9 aa, Artificial Sequence
  Description of Artificial Sequence  Synthetic Peptide
GLDTYRGSP

SEQ ID NO: 518 - protein, 6 aa, Artificial Sequence
  Description of Artificial Sequence  Synthetic Peptide
GLPSGL

SEQ ID NO: 519 - protein, 7 aa, Artificial Sequence
  Description of Artificial Sequence  Synthetic Peptide
GLPSGLL

SEQ ID NO: 520 - protein, 6 aa, Artificial Sequence
  Description of Artificial Sequence  Synthetic Peptide
GLSARH

SEQ ID NO: 521 - protein, 4 aa, Artificial Sequence
  Description of Artificial Sequence  Synthetic Peptide
GMWG

SEQ ID NO: 522 - protein, 6 aa, Artificial Sequence
  Description of Artificial Sequence  Synthetic Peptide
GSRRTP

SEQ ID NO: 523 - protein, 7 aa, Artificial Sequence
  Description of Artificial Sequence  Synthetic Peptide
GSRIRTP

SEQ ID NO: 524 - protein, 7 aa, Artificial Sequence
  Description of Artificial Sequence  Synthetic Peptide
GWTVRDG

SEQ ID NO: 525 - protein, 7 aa, Artificial Sequence
  Description of Artificial Sequence  Synthetic Peptide
GVMLRRG

SEQ ID NO: 526 - protein, 4 aa, Artificial Sequence
  Description of Artificial Sequence  Synthetic Peptide
HALE

SEQ ID NO: 527 - protein, 9 aa, Artificial Sequence
  Description of Artificial Sequence  Synthetic Peptide
CAHHALMEC

SEQ ID NO: 528 - protein, 7 aa, Artificial Sequence
  Description of Artificial Sequence  Synthetic Peptide
HGQGVRP

SEQ ID NO: 529 - protein, 7 aa, Artificial Sequence
  Description of Artificial Sequence  Synthetic Peptide
HGGSANP

SEQ ID NO: 530 - protein, 6 aa, Artificial Sequence
  Description of Artificial Sequence  Synthetic Peptide
HLSDMS

SEQ ID NO: 531 - protein, 7 aa, Artificial Sequence
  Description of Artificial Sequence  Synthetic Peptide
IAWRLEH

SEQ ID NO: 532 - protein, 4 aa, Artificial Sequence
  Description of Artificial Sequence  Synthetic Peptide
ISGL

SEQ ID NO: 533 - protein, 7 aa, Artificial Sequence
  Description of Artificial Sequence  Synthetic Peptide
ITRWRYL

SEQ ID NO: 534 - protein, 7 aa, Artificial Sequence
  Description of Artificial Sequence  Synthetic Peptide
IWSTSER

SEQ ID NO: 535 - protein, 7 aa, Artificial Sequence
  Description of Artificial Sequence  Synthetic Peptide
LRDGNGE

SEQ ID NO: 536 - protein, 6 aa, Artificial Sequence
  Description of Artificial Sequence  Synthetic Peptide
LRLLIS

SEQ ID NO: 537 - protein, 7 aa, Artificial Sequence
  Description of Artificial Sequence  Synthetic Peptide
LQHWLLS

SEQ ID NO: 538 - protein, 9 aa, Artificial Sequence
  Description of Artificial Sequence  Synthetic Peptide
CAHFIINSC

SEQ ID NO: 539 - protein, 7 aa, Artificial Sequence
  Description of Artificial Sequence  Synthetic Peptide
LEFNLGY

SEQ ID NO: 540 - protein, 6 aa, Artificial Sequence
  Description of Artificial Sequence  Synthetic Peptide
LGGLSA

SEQ ID NO: 541 - protein, 7 aa, Artificial Sequence
  Description of Artificial Sequence  Synthetic Peptide
LGLRSVG

SEQ ID NO: 542 - protein, 7 aa, Artificial Sequence
  Description of Artificial Sequence  Synthetic Peptide
LGSNSKA

SEQ ID NO: 543 - protein, 6 aa, Artificial Sequence
  Description of Artificial Sequence  Synthetic Peptide
LISGVL

SEQ ID NO: 544 - protein, 6 aa, Artificial Sequence
  Description of Artificial Sequence  Synthetic Peptide
LLSLEN

SEQ ID NO: 545 - protein, 7 aa, Artificial Sequence
  Description of Artificial Sequence  Synthetic Peptide
LVGKWPY

SEQ ID NO: 546 - protein, 9 aa, Artificial Sequence
  Description of Artificial Sequence  Synthetic Peptide
KQFSYRYLL

SEQ ID NO: 547 - protein, 10 aa, Artificial Sequence
  Description of Artificial Sequence  Synthetic Peptide
KECQSRLSCP

SEQ ID NO: 548 - protein, 7 aa, Artificial Sequence
  Description of Artificial Sequence  Synthetic Peptide
KGGRAKD

SEQ ID NO: 549 - protein, 8 aa, Artificial Sequence
  Description of Artificial Sequence  Synthetic Peptide
CAIHDSEC

SEQ ID NO: 550 - protein, 9 aa, Artificial Sequence
  Description of Artificial Sequence  Synthetic Peptide
KVSWYLDNG

SEQ ID NO: 551 - protein, 7 aa, Artificial Sequence
  Description of Artificial Sequence  Synthetic Peptide
MGDTGHC

SEQ ID NO: 552 - protein, 9 aa, Artificial Sequence
  Description of Artificial Sequence  Synthetic Peptide
MLLGYRFEK

SEQ ID NO: 553 - protein, 4 aa, Artificial Sequence
  Description of Artificial Sequence  Synthetic Peptide
MSAG

SEQ ID NO: 554 - protein, 5 aa, Artificial Sequence
  Description of Artificial Sequence  Synthetic Peptide
MVYFG

SEQ ID NO: 555 - protein, 7 aa, Artificial Sequence
  Description of Artificial Sequence  Synthetic Peptide
FDGQDRS

SEQ ID NO: 556 - protein, 4 aa, Artificial Sequence
  Description of Artificial Sequence  Synthetic Peptide
FHDS

SEQ ID NO: 557 - protein, 7 aa, Artificial Sequence
  Description of Artificial Sequence  Synthetic Peptide
FFLRDWF

SEQ ID NO: 558 - protein, 4 aa, Artificial Sequence
  Description of Artificial Sequence  Synthetic Peptide
FSVT

SEQ ID NO: 559 - protein, 4 aa, Artificial Sequence
  Description of Artificial Sequence  Synthetic Peptide
PGWV

SEQ ID NO: 560 - protein, 7 aa, Artificial Sequence
  Description of Artificial Sequence  Synthetic Peptide
CAISVGC

SEQ ID NO: 561 - protein, 7 aa, Artificial Sequence
  Description of Artificial Sequence  Synthetic Peptide
PLLVRTV

SEQ ID NO: 562 - protein, 7 aa, Artificial Sequence
  Description of Artificial Sequence  Synthetic Peptide
PLFWPYS

SEQ ID NO: 563 - protein, 6 aa, Artificial Sequence
  Description of Artificial Sequence  Synthetic Peptide
PLSLFR

SEQ ID NO: 564 - protein, 4 aa, Artificial Sequence
  Description of Artificial Sequence  Synthetic Peptide
PFHD

SEQ ID NO: 565 - protein, 5 aa, Artificial Sequence
  Description of Artificial Sequence  Synthetic Peptide
PFHDS

SEQ ID NO: 566 - protein, 7 aa, Artificial Sequence
  Description of Artificial Sequence  Synthetic Peptide
PSRRLGS

SEQ ID NO: 567 - protein, 7 aa, Artificial Sequence
  Description of Artificial Sequence  Synthetic Peptide
PSERSPS

SEQ ID NO: 568 - protein, 9 aa, Artificial Sequence
  Description of Artificial Sequence  Synthetic Peptide
PTCAYGWCA

SEQ ID NO: 569 - protein, 8 aa, Artificial Sequence
  Description of Artificial Sequence  Synthetic Peptide
SDNRYGSW

SEQ ID NO: 570 - protein, 9 aa, Artificial Sequence
  Description of Artificial Sequence  Synthetic Peptide
SDWYYPWSW

SEQ ID NO: 571 - protein, 9 aa, Artificial Sequence
  Description of Artificial Sequence  Synthetic Peptide
CALGRWDRC

SEQ ID NO: 572 - protein, 7 aa, Artificial Sequence
  Description of Artificial Sequence  Synthetic Peptide
SQHVVSG

SEQ ID NO: 573 - protein, 6 aa, Artificial Sequence
  Description of Artificial Sequence  Synthetic Peptide
SQLAAG

SEQ ID NO: 574 - protein, 6 aa, Artificial Sequence
  Description of Artificial Sequence  Synthetic Peptide
SQLVAG

SEQ ID NO: 575 - protein, 5 aa, Artificial Sequence
  Description of Artificial Sequence  Synthetic Peptide
SQPLG

SEQ ID NO: 576 - protein, 5 aa, Artificial Sequence
  Description of Artificial Sequence  Synthetic Peptide
SQPWG

SEQ ID NO: 577 - protein, 7 aa, Artificial Sequence
  Description of Artificial Sequence  Synthetic Peptide
SGKDRRS

SEQ ID NO: 578 - protein, 7 aa, Artificial Sequence
  Description of Artificial Sequence  Synthetic Peptide
SLEAFFL

SEQ ID NO: 579 - protein, 7 aa, Artificial Sequence
  Description of Artificial Sequence  Synthetic Peptide
SLGGMSG

SEQ ID NO: 580 - protein, 7 aa, Artificial Sequence
  Description of Artificial Sequence  Synthetic Peptide
SLHRVAR

SEQ ID NO: 581 - protein, 6 aa, Artificial Sequence
  Description of Artificial Sequence  Synthetic Peptide
SLLAAG

SEQ ID NO: 582 - protein, 9 aa, Artificial Sequence
  Description of Artificial Sequence  Synthetic Peptide
CALPGGQIC

SEQ ID NO: 583 - protein, 6 aa, Artificial Sequence
  Description of Artificial Sequence  Synthetic Peptide
SLLVAG

SEQ ID NO: 584 - protein, 6 aa, Artificial Sequence
  Description of Artificial Sequence  Synthetic Peptide
SKHALE

SEQ ID NO: 585 - protein, 6 aa, Artificial Sequence
  Description of Artificial Sequence  Synthetic Peptide
SMDQLP

SEQ ID NO: 586 - protein, 6 aa, Artificial Sequence
  Description of Artificial Sequence  Synthetic Peptide
SMPSLP

SEQ ID NO: 587 - protein, 7 aa, Artificial Sequence
  Description of Artificial Sequence  Synthetic Peptide
SMSIARL

SEQ ID NO: 588 - protein, 4 aa, Artificial Sequence
  Description of Artificial Sequence  Synthetic Peptide
SFSV

SEQ ID NO: 589 - protein, 5 aa, Artificial Sequence
  Description of Artificial Sequence  Synthetic Peptide
SFSVT

SEQ ID NO: 590 - protein, 4 aa, Artificial Sequence
  Description of Artificial Sequence  Synthetic Peptide
SPFH

SEQ ID NO: 591 - protein, 5 aa, Artificial Sequence
  Description of Artificial Sequence  Synthetic Peptide
SPFHD

SEQ ID NO: 592 - protein, 6 aa, Artificial Sequence
  Description of Artificial Sequence  Synthetic Peptide
SPFHDS

SEQ ID NO: 593 - protein, 9 aa, Artificial Sequence
  Description of Artificial Sequence  Synthetic Peptide
CALSAYRVC

SEQ ID NO: 594 - protein, 4 aa, Artificial Sequence
  Description of Artificial Sequence  Synthetic Peptide
SSFS

SEQ ID NO: 595 - protein, 5 aa, Artificial Sequence
  Description of Artificial Sequence  Synthetic Peptide
SSFSV

SEQ ID NO: 596 - protein, 6 aa, Artificial Sequence
  Description of Artificial Sequence  Synthetic Peptide
SSFSVT

SEQ ID NO: 597 - protein, 7 aa, Artificial Sequence
  Description of Artificial Sequence  Synthetic Peptide
SSPRPGV

SEQ ID NO: 598 - protein, 4 aa, Artificial Sequence
  Description of Artificial Sequence  Synthetic Peptide
SSPF

SEQ ID NO: 599 - protein, 5 aa, Artificial Sequence
  Description of Artificial Sequence  Synthetic Peptide
SSPFH

SEQ ID NO: 600 - protein, 6 aa, Artificial Sequence
  Description of Artificial Sequence  Synthetic Peptide
SSPFHD

SEQ ID NO: 601 - protein, 7 aa, Artificial Sequence
  Description of Artificial Sequence  Synthetic Peptide
SSPFHDS

SEQ ID NO: 602 - protein, 4 aa, Artificial Sequence
  Description of Artificial Sequence  Synthetic Peptide
SSSF

SEQ ID NO: 603 - protein, 5 aa, Artificial Sequence
  Description of Artificial Sequence  Synthetic Peptide
SSSFS

SEQ ID NO: 604 - protein, 9 aa, Artificial Sequence
  Description of Artificial Sequence  Synthetic Peptide
CALVNVHLC

SEQ ID NO: 605 - protein, 6 aa, Artificial Sequence
  Description of Artificial Sequence  Synthetic Peptide
SSSFSV

SEQ ID NO: 606 - protein, 7 aa, Artificial Sequence
  Description of Artificial Sequence  Synthetic Peptide
SSSFSVT

SEQ ID NO: 607 - protein, 7 aa, Artificial Sequence
  Description of Artificial Sequence  Synthetic Peptide
SWGSRIR

SEQ ID NO: 608 - protein, 6 aa, Artificial Sequence
  Description of Artificial Sequence  Synthetic Peptide
SWYSQF

SEQ ID NO: 609 - protein, 7 aa, Artificial Sequence
  Description of Artificial Sequence  Synthetic Peptide
SYQSPGP

SEQ ID NO: 610 - protein, 6 aa, Artificial Sequence
  Description of Artificial Sequence  Synthetic Peptide
SVRHRE

SEQ ID NO: 611 - protein, 5 aa, Artificial Sequence
  Description of Artificial Sequence  Synthetic Peptide
SVWFG

SEQ ID NO: 612 - protein, 7 aa, Artificial Sequence
  Description of Artificial Sequence  Synthetic Peptide
TRNTGNI

SEQ ID NO: 613 - protein, 7 aa, Artificial Sequence
  Description of Artificial Sequence  Synthetic Peptide
TREVHRS

SEQ ID NO: 614 - protein, 7 aa, Artificial Sequence
  Description of Artificial Sequence  Synthetic Peptide
TGVALQS

SEQ ID NO: 615 - protein, 9 aa, Artificial Sequence
  Description of Artificial Sequence  Synthetic Peptide
CALVMVGAC

SEQ ID NO: 616 - protein, 9 aa, Artificial Sequence
  Description of Artificial Sequence  Synthetic Peptide
TLRKYFHSS

SEQ ID NO: 617 - protein, 9 aa, Artificial Sequence
  Description of Artificial Sequence  Synthetic Peptide
TMLRYFMFP

SEQ ID NO: 618 - protein, 9 aa, Artificial Sequence
  Description of Artificial Sequence  Synthetic Peptide
TMLRYTVRL

SEQ ID NO: 619 - protein, 7 aa, Artificial Sequence
  Description of Artificial Sequence  Synthetic Peptide
TPKTSVT

SEQ ID NO: 620 - protein, 6 aa, Artificial Sequence
  Description of Artificial Sequence  Synthetic Peptide
TSRFSL

SEQ ID NO: 621 - protein, 7 aa, Artificial Sequence
  Description of Artificial Sequence  Synthetic Peptide
WQRLYAW

SEQ ID NO: 622 - protein, 6 aa, Artificial Sequence
  Description of Artificial Sequence  Synthetic Peptide
WGESRL

SEQ ID NO: 623 - protein, 6 aa, Artificial Sequence
  Description of Artificial Sequence  Synthetic Peptide
WGPKRL

SEQ ID NO: 624 - protein, 9 aa, Artificial Sequence
  Description of Artificial Sequence  Synthetic Peptide
WSCARPLCG

SEQ ID NO: 625 - protein, 7 aa, Artificial Sequence
  Description of Artificial Sequence  Synthetic Peptide
WYTVMSW

SEQ ID NO: 626 - protein, 8 aa, Artificial Sequence
  Description of Artificial Sequence  Synthetic Peptide
CAMGSPEC

SEQ ID NO: 627 - protein, 7 aa, Artificial Sequence
  Description of Artificial Sequence  Synthetic Peptide
WVSPVLG

SEQ ID NO: 628 - protein, 5 aa, Artificial Sequence
  Description of Artificial Sequence  Synthetic Peptide
YRARG

SEQ ID NO: 629 - protein, 5 aa, Artificial Sequence
  Description of Artificial Sequence  Synthetic Peptide
YRLRG

SEQ ID NO: 630 - protein, 9 aa, Artificial Sequence
  Description of Artificial Sequence  Synthetic Peptide
YDWWYPWSW

SEQ ID NO: 631 - protein, 9 aa, Artificial Sequence
  Description of Artificial Sequence  Synthetic Peptide
YEWWYWSWA

SEQ ID NO: 632 - protein, 7 aa, Artificial Sequence
  Description of Artificial Sequence  Synthetic Peptide
YGNDRRN

SEQ ID NO: 633 - protein, 7 aa, Artificial Sequence
  Description of Artificial Sequence  Synthetic Peptide
YIRPFTL

SEQ ID NO: 634 - protein, 7 aa, Artificial Sequence
  Description of Artificial Sequence  Synthetic Peptide
YIRPFTL

SEQ ID NO: 635 - protein, 7 aa, Artificial Sequence
  Description of Artificial Sequence  Synthetic Peptide
YICPGPC

SEQ ID NO: 636 - protein, 7 aa, Artificial Sequence
  Description of Artificial Sequence  Synthetic Peptide
YSLRIGL

SEQ ID NO: 637 - protein, 9 aa, Artificial Sequence
  Description of Artificial Sequence  Synthetic Peptide
CAFNNDDVC

SEQ ID NO: 638 - protein, 7 aa, Artificial Sequence
  Description of Artificial Sequence  Synthetic Peptide
YVQSREG

SEQ ID NO: 639 - protein, 7 aa, Artificial Sequence
  Description of Artificial Sequence  Synthetic Peptide
VRGLAAA

SEQ ID NO: 640 - protein, 7 aa, Artificial Sequence
  Description of Artificial Sequence  Synthetic Peptide
VQGIGRL

SEQ ID NO: 641 - protein, 7 aa, Artificial Sequence
  Description of Artificial Sequence  Synthetic Peptide
VGLHARA

SEQ ID NO: 642 - protein, 7 aa, Artificial Sequence
  Description of Artificial Sequence  Synthetic Peptide
VGSGRLS

SEQ ID NO: 643 - protein, 6 aa, Artificial Sequence
  Description of Artificial Sequence  Synthetic Peptide
VHALES

SEQ ID NO: 644 - protein, 7 aa, Artificial Sequence
  Description of Artificial Sequence  Synthetic Peptide
VLGPRAM

SEQ ID NO: 645 - protein, 7 aa, Artificial Sequence
  Description of Artificial Sequence  Synthetic Peptide
VMGGVVS

SEQ ID NO: 646 - protein, 7 aa, Artificial Sequence
  Description of Artificial Sequence  Synthetic Peptide
VMGSVTG

SEQ ID NO: 647 - protein, 6 aa, Artificial Sequence
  Description of Artificial Sequence  Synthetic Peptide
VMGVIA

SEQ ID NO: 648 - protein, 9 aa, Artificial Sequence
  Description of Artificial Sequence  Synthetic Peptide
CAPMEWSVC

SEQ ID NO: 649 - protein, 6 aa, Artificial Sequence
  Description of Artificial Sequence  Synthetic Peptide
VFALEG

SEQ ID NO: 650 - protein, 7 aa, Artificial Sequence
  Description of Artificial Sequence  Synthetic Peptide
VPELGHE

SEQ ID NO: 651 - protein, 7 aa, Artificial Sequence
  Description of Artificial Sequence  Synthetic Peptide
VSFLEYR

SEQ ID NO: 652 - protein, 9 aa, Artificial Sequence
  Description of Artificial Sequence  Synthetic Peptide
VVISYRYLL

SEQ ID NO: 653 - protein, 9 aa, Artificial Sequence
  Description of Artificial Sequence  Synthetic Peptide
VVISYSMDP

SEQ ID NO: 654 - protein, 9 aa, Artificial Sequence
  Description of Artificial Sequence  Synthetic Peptide
VVISYSMPD

SEQ ID NO: 655 - protein, 8 aa, Artificial Sequence
  Description of Artificial Sequence  Synthetic Peptide
CASGSENC

SEQ ID NO: 656 - protein, 8 aa, Artificial Sequence
  Description of Artificial Sequence  Synthetic Peptide
CASPNLAC

SEQ ID NO: 657 - protein, 8 aa, Artificial Sequence
  Description of Artificial Sequence  Synthetic Peptide
CASSHAVC

SEQ ID NO: 658 - protein, 8 aa, Artificial Sequence
  Description of Artificial Sequence  Synthetic Peptide
CATEAVGC

SEQ ID NO: 659 - protein, 9 aa, Artificial Sequence
  Description of Artificial Sequence  Synthetic Peptide
CAWWRFGLC

SEQ ID NO: 660 - protein, 9 aa, Artificial Sequence
  Description of Artificial Sequence  Synthetic Peptide
CAYTNSLRC

SEQ ID NO: 661 - protein, 13 aa, Artificial Sequence
  Description of Artificial Sequence  Synthetic Peptide
CAVACWADCQLGC

SEQ ID NO: 662 - protein, 8 aa, Artificial Sequence
  Description of Artificial Sequence  Synthetic Peptide
CAVKSVTC

SEQ ID NO: 663 - protein, 9 aa, Artificial Sequence
  Description of Artificial Sequence  Synthetic Peptide
CAVVRNQEC

SEQ ID NO: 664 - protein, 8 aa, Artificial Sequence
  Description of Artificial Sequence  Synthetic Peptide
CAVVFSQC

SEQ ID NO: 665 - protein, 7 aa, Artificial Sequence
  Description of Artificial Sequence  Synthetic Peptide
CRAREMC

SEQ ID NO: 666 - protein, 9 aa, Artificial Sequence
  Description of Artificial Sequence  Synthetic Peptide
CRARGWLLC

SEQ ID NO: 667 - protein, 9 aa, Artificial Sequence
  Description of Artificial Sequence  Synthetic Peptide
CRADSEGVC

SEQ ID NO: 668 - protein, 8 aa, Artificial Sequence
  Description of Artificial Sequence  Synthetic Peptide
CRASAMVC

SEQ ID NO: 669 - protein, 9 aa, Artificial Sequence
  Description of Artificial Sequence  Synthetic Peptide
CRRDTYGRC

SEQ ID NO: 670 - protein, 12 aa, Artificial Sequence
  Description of Artificial Sequence  Synthetic Peptide
CRNAWNKHGSRC

SEQ ID NO: 671 - protein, 12 aa, Artificial Sequence
  Description of Artificial Sequence  Synthetic Peptide
CRDDAKVMRYNC

SEQ ID NO: 672 - protein, 8 aa, Artificial Sequence
  Description of Artificial Sequence  Synthetic Peptide
CRDGYHHC

SEQ ID NO: 673 - protein, 8 aa, Artificial Sequence
  Description of Artificial Sequence  Synthetic Peptide
CRECGERC

SEQ ID NO: 674 - protein, 9 aa, Artificial Sequence
  Description of Artificial Sequence  Synthetic Peptide
CREQASTGC

SEQ ID NO: 675 - protein, 9 aa, Artificial Sequence
  Description of Artificial Sequence  Synthetic Peptide
CREQRHNLC

SEQ ID NO: 676 - protein, 9 aa, Artificial Sequence
  Description of Artificial Sequence  Synthetic Peptide
CREWMAGGC

SEQ ID NO: 677 - protein, 9 aa, Artificial Sequence
  Description of Artificial Sequence  Synthetic Peptide
CRGDTKALC

SEQ ID NO: 678 - protein, 9 aa, Artificial Sequence
  Description of Artificial Sequence  Synthetic Peptide
CRGEWGMMC

SEQ ID NO: 679 - protein, 8 aa, Artificial Sequence
  Description of Artificial Sequence  Synthetic Peptide
CRGGGRLC

SEQ ID NO: 680 - protein, 7 aa, Artificial Sequence
  Description of Artificial Sequence  Synthetic Peptide
CRGSAVC

SEQ ID NO: 681 - protein, 9 aa, Artificial Sequence
  Description of Artificial Sequence  Synthetic Peptide
CRGWADRKC

SEQ ID NO: 682 - protein, 9 aa, Artificial Sequence
  Description of Artificial Sequence  Synthetic Peptide
CRHQSASAC

SEQ ID NO: 683 - protein, 12 aa, Artificial Sequence
  Description of Artificial Sequence  Synthetic Peptide
CRIRGIQLRPAC

SEQ ID NO: 684 - protein, 9 aa, Artificial Sequence
  Description of Artificial Sequence  Synthetic Peptide
CRIRMSAGC

SEQ ID NO: 685 - protein, 9 aa, Artificial Sequence
  Description of Artificial Sequence  Synthetic Peptide
CRLADKELC